SEQUENCE LISTING (489 sequences)

SEQ ID NO: 1 - DNA, 2820 bp, Homo sapiens
gggagttcagacctagatctttccagttaatcacacaacaaacttagctcatcgcaataaaaagcagctcagagccgactggctcttttaggcactgactccgaacaggattctttcacccaggcatctcctccagagggatccgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagctgaagtaagtgtgtctgaagcacccaccaatacaactgtcttttacatgaagatttcagagaatgtggaatttaaaatgtcacttacaacaatcctaagacaactactggagagtcatgtttgttgaaattctcattaatgtttatgggtgttttctgttgttttgtttgtcagtgttattttgtcaatgttgaagtgaattaaggtacatgcaagtgtaataacatatctcctgaagatacttgaatggattaaaaaaacacacaggtatatttgctggatgataaagatttcatgggaaaaaaaatcaattaatctgtctaagctgctttctgatgttggtttcttaataatgagtaaaccacaaattaaatgttattttaacctcaccaaaacaatttataccttgtgtccctaaattgtagccctatattaaattatattacatttcaaaaaaaaaaaaaaaa

SEQ ID NO: 2 - protein, 118 aa, Artificial Sequence
  source: /note=“Description of Artificial Sequence: Synthetic polypeptide”
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 3 - protein, 112 aa, Artificial Sequence
  source: /note=“Description of Artificial Sequence: Synthetic polypeptide”
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKR

SEQ ID NO: 4 - protein, 12 aa, Artificial Sequence
  source: /note=“Description of Artificial Sequence: Synthetic peptide”
PKSCDCHCPPCP

SEQ ID NO: 5 - protein, 100 aa, Artificial Sequence
  source: /note=“Description of Artificial Sequence: Synthetic polypeptide”
  MISC_FEATURE (1)..(100): /note=“This region may encompass 1-20 ′Gly-Gly-Gly-Gly-Ser′ repeating units”
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 6 - DNA, 2306 bp, Homo sapiens
actgactccgaacaggattctttcacccaggcatctcctccagagggatccgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagctgaagtaagtgtgtctgaagcacccaccaatacaactgtcttttac

SEQ ID NO: 7 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 8 - DNA, 1731 bp, Homo sapiens
aactgcagcgccggggctgggggaggggagcctactcactcccccaactcccgggcggtgactcatcaacgagcaccagcggccagaggtgagcagtcccgggaaggggccgagaggcggggccgccaggtcgggcaggtgtgcgctccgccccgccgcgcgcacagagcgctagtccttcggcgagcgagcaccttcgacgcggtccggggaccccctcgtcgctgtcctcccgacgcggacccgcgtgccccaggcctcgcgctgcccggccggctcctcgtgtcccactcccggcgcacgccctcccgcgagtcccgggcccctcccgcgcccctcttctcggcgcgcgcgcagcatggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccatgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcataactatataatttgaagattatagaagaagggaaatagcaaatggacacaaattacaaatgtgtgtgcgtgggacgaagacatctttgaaggtcatgagtttgttagtttaacatcatatatttgtaatagtgaaacctgtactcaaaatataagcagcttgaaactggctttaccaatcttgaaatttgaccacaagtgtcttatatatgcagatctaatgtaaaatccagaacttggactccatcgttaaaattatttatgtgtaacattcaaatgtgtgcattaaatatgcttccacagtaaaatctgaaaaactgatttgtgattgaaagctgcctttctatttacttgagtcttgtacatacatacttttttatgagctatgaaataaaacattttaaactgaatttcttaaaaaaaaaaaaaaa

SEQ ID NO: 9 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 10-399 - skipped (390 entries)

SEQ ID NO: 400 - protein, 214 aa, Artificial Sequence
  source: /note=“Description of Artificial Sequence: Synthetic polypeptide”
DIQMTQSPSSVSASVGDRVTITCRASQGISSWLAWYQQKPGKAPKLLIYAASSLQSGVPSRFSGSGSGTDFTLTISSLQPEDFATYYCLQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 401 - skipped

SEQ ID NO: 402 - DNA, 648 bp, Artificial Sequence
  source: /note=“Description of Artificial Sequence: Synthetic polynucleotide”
gacattcagatgacccagtctccttcaagcgtcagcgcttccgtgggcgaccgggtcaccatcacatgcagagcctcccaggggattagctcctggctggcttggtatcagcagaagcctgggaaagcaccaaagctgctgatctatgccgcttctagtctgcagtccggagtgccctctcgattctctggcagtgggtcaggaaccgactttactctgaccatttcaagcctgcagcctgaggatttcgctacatactattgcctgcaggcaaactctttcccccctacctttggcgggggaacaaaagtggagatcaagcgtacggtggcagccccatccgtcttcatttttccaccctctgacgaacagctgaaaagtggcacagccagcgtggtctgtctgctgaacaatttttacccccgcgaagccaaagtgcagtggaaggtcgataacgctctgcagagcgggaattcccaggagtctgtgactgaacaggacagtaaagattcaacctatagcctgtcctctacactgactctgagcaaggcagattacgagaagcacaaagtgtatgcctgcgaagtcacacatcagggactgagttcacctgtgactaagagcttcaatagaggcgagtgttgataa

SEQ ID NO: 403 - protein, 973 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPLSLPVTPGEPASISCRSTKSLLHSNGITYLYWYLQKPGQSPQLLIYQMSNLASGVPDRFSSSGSGTDFTLKISRVEAEDEGVYYCAQNLEIPRTFGGGTKLEIKRTGGGGSGGGGSGGGGSGGGGSQVQLVQSGAEVKKPGESVKISCKASGYTFTNYGMNWVRQAPGQGLKWMGWINTYTGESTYADDFKGRFAFSLDTSASTAYLQLSSLRSEDTAVYFCARFAIKGDYWGQGTLVTVSS

SEQ ID NO: 404 - protein, 974 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSQVQLVQSGSELKKPGESVKVSCKASGYTFTNYGMHWVRQAPGQGLKWMGWINTYTGESTYADDFKGRFAFSLDTSASTAYLQISSLKAEDTAVYFCARFAIKGDYWGQGTLVTVSSASTGGGGSGGGGSGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPYTFGQGTKVEIK

SEQ ID NO: 405 - protein, 974 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSQVQLVQSGSELKKPGESVKVSCKASGYTFTNYGMNWVRQAPGQGLKWMGWINTYTGESTYADDFKGRFAFSLDTSASTAYLQISSLKAEDTAVYFCARFAIKGDYWGQGTLVTVSSASTGGGGSGGGGSGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGQGTKVEIK

SEQ ID NO: 406 - protein, 974 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGQGTKVEIKRTGGGGSGGGGSGGGGSGGGGSGQVQLVQSGSELKKPGESVKVSCKASGYTFTNYGMNWVRQAPGQGLKWMGWINTYTGESTYADDFKGRFAFSLDTSASTAYLQISSLKAEDTAVYFCARFAIKGDYWGQGTLVTVSS

SEQ ID NO: 407 - protein, 973 aa, Artificial Sequence; identical to SEQ ID NO: 403
  source: /note=“Description of Artificial Sequence: Synthetic polypeptide”

SEQ ID NO: 408 - DNA, 2925 bp, Artificial Sequence
  source: /note=“Description of Artificial Sequence: Synthetic polynucleotide”
cagctgcagctgcaggagagcggacccggactggtcaagccctccgagacactgtctctgacctgcacagtgtcaggcgggagcatcagctcctctgtctactattggtcttggattcggcagccccctggaaagggcctggaatggatcggcgtgatctacccaagtgggaacacttactattctcccagtctgaaatccagagtgaccatcagtgtcgacacatcaaagaatcagttcagcctgaaactgagttcagtgacagccgctgatactgcagtctactattgtgccaggaccatctatgacctgtttgatatttgggggcagggaactatggtgaccgtcagctccgctagcactaagggaccttccgtgttcccactggccccctcaagcaaatctaccagtgggggaacagcagccctgggctgtctggtgaaggattactttccagagcccgtgaccgtcagttggaactcaggggctctgactagcggagtgcacaccttccccgcagtcctgcagtcctctgggctgtactccctgagttcagtggtcactgtgcccagctcctctctgggaacacagacttatatctgcaacgtgaatcacaagccttccaataccaaagtcgacaagaaagtcgagcctaagtcatgtgataaaacccatacatgcccaagctgtcctgcaccagaatttctgggaggaccaagcgtgttcctgtttccacctaagcctaaagacaccctgatgattagtcgcactccagaggtgacctgtgtggtcgtggacgtcagccaggaggatcccgaagtgcagtttaactggtacgtcgatggcgtggaagtccacaatgctaagacaaaaccaagagaggaacagttcaacagcaagtacagggtcgtgtccgtgctgactgtcctgcatcaggactggctgaacggaaaagagtataagtgcaaagtgagcaataagggcctgcccagttcaatcgagaaaacaatttccaaggcaaaaggccagcctcgggaaccacaggtgtataccctgccacccagcagagaggaaatgacaaagaaccaggtgtcactgagctgtgccgtcaaaggcttttaccctagcgatatcgctgtggagtgggaatccaatgggcagccagaaaacaattataagaccacacctccagtgctggacagcgatggatccttctttctggtgtccaagctgaccgtggacaaatctcggtggcagcagggcaacgtgttctcctgctctgtcatgcatgaggccctgcacaatcattacacccagaaaagtctgtcactgagcaagtcctgtgataaaacaggaggcgggggatctggcgggggaggcagtgggggaggcgggtccggaggaggaggaagcggaggaggaggatccggaggaggcgggtctggaggcgggggaagtggaggaggaggatcttgcccaagttgtccagcacctgagttcctgggaggaccatccgtgttcctgtttccccctaagcccaaagacaccctgatgatcagccggacacctgaagtgacttgcgtcgtggtggacgtgtcccaggaggatccagaggtccagtttaactggtatgtcgatggcgtggaggtccacaatgctaagaccaaacctcgcgaggaacagttcaactctgactaccgagtggtcagtgtgctgacagtcctgcatcaggattggctgaacgggaaggaatacaaatgtaaagtgagcaataagggactgccaagctccatcgagaagaccatcagcaaagccaaaggccagcccagggaacctcaggtgtacactctgccaccctcccgcgaggaaatgaccaagaaccaggtgtctctgtggtgtctggtcaaaggattttatccctctgacatcgccgtggagtgggaaagtaatggccagcctgaaaacaattacaagactacccctccagtgctggactcagatgggagcttctttctgtatagtaagctgaccgtggataaatcacggtggcagcagggaaatgtgttctcttgcagtgtcatgcacgaggccctgcataaccattacacacagaagtcattaagcttatcgggggaatgtggaggcggagggagcggcggaggcggcagcgatatcgtgatgacacagtcacccctgtcgctccctgtgactcccggagagcctgcgtccatctcgtgccggtcaacaaagtccctgctgcacagcaatggaattacgtatttgtactggtatttgcaaaaacccggacagagcccgcaattgttgatctaccagatgtcaaaccttgcgagcggggtcccggatcgctttagctcgtcggggtcaggaactgacttcacactcaaaatctcaagggtcgaggccgaggatgaaggtgtctattactgcgctcagaatctcgaaatcccgcggacctttggtggaggtacgaagctggagatcaagcgaacgggaggcggtgggtccggtggcggtggttccggcggaggtggttcgggaggaggaggtagccaggtgcagcttgtccagtcgggagcagaggtaaagaagccaggagagtcggtgaaaatctcgtgtaaagcctcggggtacacattcacgaactacggcatgaattgggtgagacaagctcccgggcagggcttgaaatggatgggatggattaacacctacacaggggaatccacatatgcggacgacttcaaggggaggttcgcgttttcacttgatacttcagcgagcacggcgtatctccaactctcgtcgcttcgctccgaagataccgcagtatacttttgcgccagattcgcgattaaaggggactattggggacaggggacccttgtcacggtgtcatcatgataa

SEQ ID NO: 409 - protein, 973 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGGGTKLEIKRTGGGGSGGGGSGGGGSGGGGSQVQLVQSGAEVKKPGESVKISCKASGYTFTNYGMNWVRQAPGQGLKWMGWINTYTGESTYADDFKGRFAFSLDTSASTAYLQLSSLRSEDTAVYFCARFAIKGDYWGQGTLVTVSS

SEQ ID NO: 410 - protein, 973 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGCGTKLEIKRTGGGGSGGGGSGGGGSGGGGSQVQLVQSGAEVKKPGESVKISCKASGYTFTNYGMNWVRQAPGQCLKWMGWINTYTGESTYADDFKGRFAFSLDTSASTAYLQLSSLRSEDTAVYFCARFAIKGDYWGQGTLVTVSS

SEQ ID NO: 411 - DNA, 2925 bp, Artificial Sequence
  source: /note=“Description of Artificial Sequence: Synthetic polynucleotide”
cagctgcagctgcaggagagcggacccggactggtcaagccctccgagacactgtctctgacctgcacagtgtcaggcgggagcatcagctcctctgtctactattggtcttggattcggcagccccctggaaagggcctggaatggatcggcgtgatctacccaagtgggaacacttactattctcccagtctgaaatccagagtgaccatcagtgtcgacacatcaaagaatcagttcagcctgaaactgagttcagtgacagccgctgatactgcagtctactattgtgccaggaccatctatgacctgtttgatatttgggggcagggaactatggtgaccgtcagctccgctagcactaagggaccttccgtgttcccactggccccctcaagcaaatctaccagtgggggaacagcagccctgggctgtctggtgaaggattactttccagagcccgtgaccgtcagttggaactcaggggctctgactagcggagtgcacaccttccccgcagtcctgcagtcctctgggctgtactccctgagttcagtggtcactgtgcccagctcctctctgggaacacagacttatatctgcaacgtgaatcacaagccttccaataccaaagtcgacaagaaagtcgagcctaagtcatgtgataaaacccatacatgcccaagctgtcctgcaccagaatttctgggaggaccaagcgtgttcctgtttccacctaagcctaaagacaccctgatgattagtcgcactccagaggtgacctgtgtggtcgtggacgtcagccaggaggatcccgaagtgcagtttaactggtacgtcgatggcgtggaagtccacaatgctaagacaaaaccaagagaggaacagttcaacagcaagtacagggtcgtgtccgtgctgactgtcctgcatcaggactggctgaacggaaaagagtataagtgcaaagtgagcaataagggcctgcccagttcaatcgagaaaacaatttccaaggcaaaaggccagcctcgggaaccacaggtgtataccctgccacccagcagagaggaaatgacaaagaaccaggtgtcactgagctgtgccgtcaaaggcttttaccctagcgatatcgctgtggagtgggaatccaatgggcagccagaaaacaattataagaccacacctccagtgctggacagcgatggatccttctttctggtgtccaagctgaccgtggacaaatctcggtggcagcagggcaacgtgttctcctgctctgtcatgcatgaggccctgcacaatcattacacccagaaaagtctgtcactgagcaagtcctgtgataaaacaggaggcgggggatctggcgggggaggcagtgggggaggcgggtccggaggaggaggaagcggaggaggaggatccggaggaggcgggtctggaggcgggggaagtggaggaggaggatcttgcccaagttgtccagcacctgagttcctgggaggaccatccgtgttcctgtttccccctaagcccaaagacaccctgatgatcagccggacacctgaagtgacttgcgtcgtggtggacgtgtcccaggaggatccagaggtccagtttaactggtatgtcgatggcgtggaggtccacaatgctaagaccaaacctcgcgaggaacagttcaactctgactaccgagtggtcagtgtgctgacagtcctgcatcaggattggctgaacgggaaggaatacaaatgtaaagtgagcaataagggactgccaagctccatcgagaagaccatcagcaaagccaaaggccagcccagggaacctcaggtgtacactctgccaccctcccgcgaggaaatgaccaagaaccaggtgtctctgtggtgtctggtcaaaggattttatccctctgacatcgccgtggagtgggaaagtaatggccagcctgaaaacaattacaagactacccctccagtgctggactcagatgggagcttctttctgtatagtaagctgaccgtggataaatcacggtggcagcagggaaatgtgttctcttgcagtgtcatgcacgaggccctgcataaccattacacacagaagtcattaagcttatcgggggaatgtggaggcggagggagcggcggaggcggcagcgatatcgtgatgacacagtcacccctgtcgctccctgtgactcccggagagcctgcgtccatctcgtgccggtcaacaaagtccctgctgcacagcgacggaattacgtatttgtactggtatttgcaaaaacccggacagagcccgcaattgttgatctaccagctctcaaaccttgcgagcggggtcccggatcgctttagctcgtcggggtcaggaactgacttcacactcaaaatctcaagggtcgaggccgaggatgaaggtgtctattactgcgctcagaatctcgaaatcccgcggacctttggttgcggtacgaagctggagatcaagcgaacgggaggcggtgggtccggtggcggtggttccggcggaggtggttcgggaggaggaggtagccaggtgcagcttgtccagtcgggagcagaggtaaagaagccaggagagtcggtgaaaatctcgtgtaaagcctcggggtacacattcacgaactacggcatgaattgggtgagacaagctcccgggcagtgtttgaaatggatgggatggattaacacctacacaggggaatccacatatgcggacgacttcaaggggaggttcgcgttttcacttgatacttcagcgagcacggcgtatctccaactgtcgtcgcttcgctccgaagataccgcagtatacttttgcgccagattcgcgattaaaggggactattggggacaggggacccttgtcacggtgtcatcatgataa

SEQ ID NO: 412 - protein, 976 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSQVQLVQSGAEVKKPGESVKISCKASGYTFTNYGMNWVRQAPGQGLKWMGWINTYTGESTYADDFKGRFAFSLDTSASTAYLQLSSLRSEDTAVYFCARFAIKGDYWGQGTLVTVSSASTGGGGSGGGGSGGGGSGGGGSDIVMTQSPLSLPVTPGEPASISCRSTKSLLHSDGITYLYWYLQKPGQSPQLLIYQMSNLASGVPDRFSSSGSGTDFTLKISRVEAEDEGVYYCAQNLEIPRTFGGGTKLEIKRT

SEQ ID NO: 413 - protein, 975 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSQVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSSASTGGGGSGGGGSGGGGSGGGGSGDIVMTQSPSSLSASVGDKATITCRSTKSLLHSNGITYLYWYLQKPGKAPKLLIYQMSNLASGVPDRFSSSGSGTEFTLTISSVQPEDEGTYYCAQNLEIPRTFGQGTKLEIK

SEQ ID NO: 414 - protein, 975 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSQVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSSASTGGGGSGGGGSGGGGSGGGGSGDIVMTQSPSSLSASVGDKATITCRSTKSLLHSDGITYLYWYLQKPGKAPKLLIYQLSNLASGVPDRFSSSGSGTEFTLTISSVQPEDEGTYYCAQNLEIPRTFGQGTKLEIK

SEQ ID NO: 415 - protein, 974 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPSSLSASVGDKATITCRSTKSLLHSNGITYLYWYLQKPGKAPKLLIYQMSNLASGVPDRFSSSGSGTEFTLTISSVQPEDEGTYYCAQNLEIPRTFGQGTKLEIKRTGGGGSGGGGSGGGGSGGGGSGQVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSS

SEQ ID NO: 416 - protein, 974 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPSSLSASVGDKATITCRSTKSLLHSDGITYLYWYLQKPGKAPKLLIYQLSNLASGVPDRFSSSGSGTEFTLTISSVQPEDEGTYYCAQNLEIPRTFGQGTKLEIKRTGGGGSGGGGSGGGGSGGGGSGQVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSS

SEQ ID NO: 417 - protein, 976 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPSSLSASVGDKATITCRSTKSLLHSNGITYLYWYLQKPGKAPKLLIYQMSNLASGVPDRFSSSGSGTEFTLTISSVQPEDEGTYYCAQNLEIPRTFGQGTKLEIKRTPSHNSHQVPSAGGPTANSGTSGSQVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSS

SEQ ID NO: 418 - DNA, 2934 bp, Artificial Sequence
  source: /note=“Description of Artificial Sequence: Synthetic polynucleotide”
cagctgcagctgcaggagagcggacccggactggtcaagccctccgagacactgtctctgacctgcacagtgtcaggcgggagcatcagctcctctgtctactattggtcttggattcggcagccccctggaaagggcctggaatggatcggcgtgatctacccaagtgggaacacttactattctcccagtctgaaatccagagtgaccatcagtgtcgacacatcaaagaatcagttcagcctgaaactgagttcagtgacagccgctgatactgcagtctactattgtgccaggaccatctatgacctgtttgatatttgggggcagggaactatggtgaccgtcagctccgctagcactaagggaccttccgtgttcccactggccccctcaagcaaatctaccagtgggggaacagcagccctgggctgtctggtgaaggattactttccagagcccgtgaccgtcagttggaactcaggggctctgactagcggagtgcacaccttccccgcagtcctgcagtcctctgggctgtactccctgagttcagtggtcactgtgcccagctcctctctgggaacacagacttatatctgcaacgtgaatcacaagccttccaataccaaagtcgacaagaaagtcgagcctaagtcatgtgataaaacccatacatgcccaagctgtcctgcaccagaatttctgggaggaccaagcgtgttcctgtttccacctaagcctaaagacaccctgatgattagtcgcactccagaggtgacctgtgtggtcgtggacgtcagccaggaggatcccgaagtgcagtttaactggtacgtcgatggcgtggaagtccacaatgctaagacaaaaccaagagaggaacagttcaacagcaagtacagggtcgtgtccgtgctgactgtcctgcatcaggactggctgaacggaaaagagtataagtgcaaagtgagcaataagggcctgcccagttcaatcgagaaaacaatttccaaggcaaaaggccagcctcgggaaccacaggtgtataccctgccacccagcagagaggaaatgacaaagaaccaggtgtcactgagctgtgccgtcaaaggcttttaccctagcgatatcgctgtggagtgggaatccaatgggcagccagaaaacaattataagaccacacctccagtgctggacagcgatggatccttctttctggtgtccaagctgaccgtggacaaatctcggtggcagcagggcaacgtgttctcctgctctgtcatgcatgaggccctgcacaatcattacacccagaaaagtctgtcactgagcaagtcctgtgataaaacaggaggcgggggatctggcgggggaggcagtgggggaggcgggtccggaggaggaggaagcggaggaggaggatccggaggaggcgggtctggaggcgggggaagtggaggaggaggatcttgcccaagttgtccagcacctgagttcctgggaggaccatccgtgttcctgtttccccctaagcccaaagacaccctgatgatcagccggacacctgaagtgacttgcgtcgtggtggacgtgtcccaggaggatccagaggtccagtttaactggtatgtcgatggcgtggaggtccacaatgctaagaccaaacctcgcgaggaacagttcaactctgactaccgagtggtcagtgtgctgacagtcctgcatcaggattggctgaacgggaaggaatacaaatgtaaagtgagcaataagggactgccaagctccatcgagaagaccatcagcaaagccaaaggccagcccagggaacctcaggtgtacactctgccaccctcccgcgaggaaatgaccaagaaccaggtgtctctgtggtgtctggtcaaaggattttatccctctgacatcgccgtggagtgggaaagtaatggccagcctgaaaacaattacaagactacccctccagtgctggactcagatgggagcttctttctgtatagtaagctgaccgtggataaatcacggtggcagcagggaaatgtgttctcttgcagtgtcatgcacgaggccctgcataaccattacacacagaagtcattaagcttatcgggagagtgcggtggcggagggagcggaggaggcgggtcggacatcgtcatgacccagtcaccgtcctcactgtcggcgtcggtgggtgataaggccacaattacatgccgcagcacgaaatcactgctccactccaatgggattacatatctctattggtatctccaaaaacccggaaaggcacctaagttgctgatctaccagatgtcgaacttggcatcgggagtacccgataggttctcgtcgtcgggaagcggcacggagttcacgctcaccatttcctcagtccagccggaggacgaaggaacttactactgcgctcagaatcttgaaatcccgcgcacatttggacaagggacgaaacttgaaatcaagcgaactccgtcccacaacagccatcaagtgccctcggcgggagggcccaccgccaattcggggacatcagggagccaggtacagttgcagcagtcgggaggcgggctggtaaaacctggtgaaagcgtcaagatctcatgtgcagcctcagggtatacgttcaccaattacgggatgaactgggtgaagcaggcgccagggaaaggtcttaagtggatgggatggatcaacacttacacgggagagtccacatacgcggatgactttaaggggcggttcacgttttcgttggagacttcagcgtccgctgcctacctccaaatcaactcccttagacccgaggacacagcggtctatttctgtgcgcggtttgccattaaaggtgattattggggacagggtacgactgtgaccgtgtccagctgataa

SEQ ID NO: 419 - protein, 943 aa, Artificial Sequence
  source: /note=“Description of Artificial Sequence: Synthetic polypeptide”
MGAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGGGGSHHHHHH

SEQ ID NO: 420 - protein, 508 aa, Artificial Sequence
  source: /note=“Description of Artificial Sequence: Synthetic polypeptide”
MGWSLILLFLVAVATRVLSQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKIEGRMDPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSHHHHHH

SEQ ID NO: 421 - protein, 119 aa, Artificial Sequence [sequence omitted]

SEQ ID NO: 422 - protein, 114 aa, Artificial Sequence
  source: /note=“Description of Artificial Sequence: Synthetic polypeptide”
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKR

SEQ ID NO: 423 - protein, 216 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 424 - skipped

SEQ ID NO: 425 - protein, 974 aa, Artificial Sequence
  source: /note=“Description of Artificial Sequence: Synthetic polypeptide”
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPLSLPVTPGEPASISCRSTKSLLHSNGITYLYWYLQKPGQSPQLLIYQMSNLASGVPDRFSSSGSGTDFTLKISRVEAEDEGVYYCAQNLEIPRTFGGGTKLEIKRTGGGGSGGGGSGGGGSGGGGSQVQLVQSGAEVKKPGESVKISCKASGYTFTNYGMNWVRQAPGQGLKWMGWINTYTGESTYADDFKGRFAFSLDTSASTAYLQLSSLRSEDTAVYFCARFAIKGDYWGQGTLVTVSS

SEQ ID NO: 426 - protein, 975 aa, Artificial Sequence
  source: /note=“Description of Artificial Sequence: Synthetic polypeptide”
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSQVQLVQSGSELKKPGESVKVSCKASGYTFTNYGMHWVRQAPGQGLKWMGWINTYTGESTYADDFKGRFAFSLDTSASTAYLQISSLKAEDTAVYFCARFAIKGDYWGQGTLVTVSSASTGGGGSGGGGSGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPYTFGQGTKVEIK

SEQ ID NO: 427 - protein, 975 aa, Artificial Sequence
  source: /note=“Description of Artificial Sequence: Synthetic polypeptide”
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSQVQLVQSGSELKKPGESVKVSCKASGYTFTNYGMNWVRQAPGQGLKWMGWINTYTGESTYADDFKGRFAFSLDTSASTAYLQISSLKAEDTAVYFCARFAIKGDYWGQGTLVTVSSASTGGGGSGGGGSGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGQGTKVEIK

SEQ ID NO: 428 - protein, 975 aa, Artificial Sequence
  source: /note=“Description of Artificial Sequence: Synthetic polypeptide”
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGQGTKVEIKRTGGGGSGGGGSGGGGSGGGGSGQVQLVQSGSELKKPGESVKVSCKASGYTFTNYGMNWVRQAPGQGLKWMGWINTYTGESTYADDFKGRFAFSLDTSASTAYLQISSLKAEDTAVYFCARFAIKGDYWGQGTLVTVSS

SEQ ID NO: 429 - protein, 974 aa, Artificial Sequence; identical to SEQ ID NO: 425
  source: /note=“Description of Artificial Sequence: Synthetic polypeptide”

SEQ ID NO: 430 - protein, 974 aa, Artificial Sequence
  source: /note=“Description of Artificial Sequence: Synthetic polypeptide”
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGGGTKLEIKRTGGGGSGGGGSGGGGSGGGGSQVQLVQSGAEVKKPGESVKISCKASGYTFTNYGMNWVRQAPGQGLKWMGWINTYTGESTYADDFKGRFAFSLDTSASTAYLQLSSLRSEDTAVYFCARFAIKGDYWGQGTLVTVSS

SEQ ID NO: 431 - protein, 974 aa, Artificial Sequence
  source: /note=“Description of Artificial Sequence: Synthetic polypeptide”
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGCGTKLEIKRTGGGGSGGGGSGGGGSGGGGSQVQLVQSGAEVKKPGESVKISCKASGYTFTNYGMNWVRQAPGQCLKWMGWINTYTGESTYADDFKGRFAFSLDTSASTAYLQLSSLRSEDTAVYFCARFAIKGDYWGQGTLVTVSS

SEQ ID NO: 432 - protein, 977 aa, Artificial Sequence
  source: /note=“Description of Artificial Sequence: Synthetic polypeptide”
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSQVQLVQSGAEVKKPGESVKISCKASGYTFTNYGMNWVRQAPGQGLKWMGWINTYTGESTYADDFKGRFAFSLDTSASTAYLQLSSLRSEDTAVYFCARFAIKGDYWGQGTLVTVSSASTGGGGSGGGGSGGGGSGGGGSDIVMTQSPLSLPVTPGEPASISCRSTKSLLHSDGITYLYWYLQKPGQSPQLLIYQMSNLASGVPDRFSSSGSGTDFTLKISRVEAEDEGVYYCAQNLEIPRTFGGGTKLEIKRT

SEQ ID NO: 433 - protein, 976 aa, Artificial Sequence
  source: /note=“Description of Artificial Sequence: Synthetic polypeptide”
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSQVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSSASTGGGGSGGGGSGGGGSGGGGSGDIVMTQSPSSLSASVGDKATITCRSTKSLLHSNGITYLYWYLQKPGKAPKLLIYQMSNLASGVPDRFSSSGSGTEFTLTISSVQPEDEGTYYCAQNLEIPRTFGQGTKLEIK

SEQ ID NO: 434 - protein, 976 aa, Artificial Sequence
  source: /note=“Description of Artificial Sequence: Synthetic polypeptide”
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSQVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSSASTGGGGSGGGGSGGGGSGGGGSGDIVMTQSPSSLSASVGDKATITCRSTKSLLHSDGITYLYWYLQKPGKAPKLLIYQLSNLASGVPDRFSSSGSGTEFTLTISSVQPEDEGTYYCAQNLEIPRTFGQGTKLEIK

SEQ ID NO: 435 - protein, 975 aa, Artificial Sequence
  source: /note=“Description of Artificial Sequence: Synthetic polypeptide”
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPSSLSASVGDKATITCRSTKSLLHSNGITYLYWYLQKPGKAPKLLIYQMSNLASGVPDRFSSSGSGTEFTLTISSVQPEDEGTYYCAQNLEIPRTFGQGTKLEIKRTGGGGSGGGGSGGGGSGGGGSGQVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSS

SEQ ID NO: 436 - protein, 975 aa, Artificial Sequence
  source: /note=“Description of Artificial Sequence: Synthetic polypeptide”
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPSSLSASVGDKATITCRSTKSLLHSDGITYLYWYLQKPGKAPKLLIYQLSNLASGVPDRFSSSGSGTEFTLTISSVQPEDEGTYYCAQNLEIPRTFGQGTKLEIKRTGGGGSGGGGSGGGGSGGGGSGQVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSS

SEQ ID NO: 437 - protein, 977 aa, Artificial Sequence
  source: /note=“Description of Artificial Sequence: Synthetic polypeptide”
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPSSLSASVGDKATITCRSTKSLLHSNGITYLYWYLQKPGKAPKLLIYQMSNLASGVPDRFSSSGSGTEFTLTISSVQPEDEGTYYCAQNLEIPRTFGQGTKLEIKRTPSHNSHQVPSAGGPTANSGTSGSQVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSS

SEQ ID NO: 438 - protein, 116 aa, Artificial Sequence
  source: /note=“Description of Artificial Sequence: Synthetic polypeptide”
QVQLVQSGAEVKKPGASVKVSCKASGYTFTNYGMNWVRQAPGQGLKWMGWINTYTGESTYADDFKGRVTITADTSASTAYMELSSLRSEDTAVYYCARFAIKGDYWGQGTLVTVSS

SEQ ID NO: 439 - DNA, 348 bp, Artificial Sequence
  source: /note=“Description of Artificial Sequence: Synthetic polynucleotide”
caagtgcagttggtccagagcggtgcggaggtaaagaaacccggtgcatccgtgaaggtgtcgtgcaaagcctccgggtatacgttcacgaactatgggatgaactgggtcagacaagcaccgggtcagggactcaaatggatggggtggatcaatacatacacaggggaatcgacctacgcggatgactttaagggaagggtcaccattacggcggacacctcggcatcgactgcgtatatggaactctcatcacttcgctcggaggacacagccgtctactattgtgcgcggtttgcgatcaagggagattactggggacagggaactttggtaacagtatcaagc

SEQ ID NO: 440 - protein, 116 aa, Artificial Sequence
  source: /note=“Description of Artificial Sequence: Synthetic polypeptide”
QVQLVQSGAEVKKPGASVKVSCKASGYTFTNYGMNWVRQAPGQGLKWMGWINTYTGESTYADDFKGRVTITLDTSASTAYMELSSLRSEDTAVYFCARFAIKGDYWGQGTLVTVSS

SEQ ID NO: 441 - DNA, 348 bp, Artificial Sequence
  source: /note=“Description of Artificial Sequence: Synthetic polynucleotide”
caagtgcagttggtccagagcggtgcggaggtaaagaaacccggtgcatccgtgaaggtgtcgtgcaaagcctccgggtatacgttcacgaactatgggatgaactgggtcagacaagcaccgggtcagggactcaaatggatggggtggatcaatacatacacaggggaatcgacctacgcggatgactttaagggaagggtcaccattacgttggacacctcggcatcgactgcgtatatggaactctcatcacttcgctcggaggacacagccgtctacttctgtgcgcggtttgcgatcaagggagattactggggacagggaactttggtaacagtatcaagc

SEQ ID NO: 442 - protein, 116 aa, Artificial Sequence
  source: /note=“Description of Artificial Sequence: Synthetic polypeptide”
QVQLQQSGGGLVKPGGSVKISCKASGYTFTNYGMNWVKQAPGKGLKWMGWINTYTGESTYADDFKGRFAFSLETSASAAYLQINSLRPEDTAVYFCARFAIKGDYWGQGTLVTVSS

SEQ ID NO: 443 - DNA, 354 bp, Artificial Sequence
  source: /note=“Description of Artificial Sequence: Synthetic polynucleotide”
caggtacagctgcagcaaagcgggggaggactcgtgaagcctggtggttcggtcaaaatctcgtgtaaagcgtcagggtacaccttcacaaactatggtatgaactgggtgaaacaggcacccggaaagggtcttaagtggatgggctggatcaatacctacacgggggagtcgacatatgcggacgactttaaaggacggttcgcgttttcgttggagactagcgcctccgctgcctacctccaaatcaatagccttaggccggaagatacggcggtctacttttgcgcaagatttgccattaagggggattattggggccaggggacgctggtgacagtcagctccgctagc

SEQ ID NO: 444 - protein, 116 aa, Artificial Sequence
  source: /note=“Description of Artificial Sequence: Synthetic polypeptide”
QVQLVQSGAEVKKPGASVKVSCKASGYTFTNYGMNWVRQAPGQGLKWMGWINTYTGESTYADDFKGRFAFSLDTSASTAYMELSSLRSEDTAVYFCARFAIKGDYWGQGTLVTVSS

SEQ ID NO: 445 - DNA, 348 bp, Artificial Sequence
  source: /note=“Description of Artificial Sequence: Synthetic polynucleotide”
caggtccagcttgtgcagtccggagccgaagtcaagaagccgggagccagcgtaaaggtgtcatgtaaggcgtcggggtatacattcacgaactacggtatgaattgggtgcgccaagctcccggacagggtttgaaatggatggggtggatcaacacgtatacaggggaatcaacttacgccgacgacttcaagggaaggttcgcattttcgttggatacatcggcgtccacggcgtacatggagctgtcaagcctgcggtcggaggacacggcggtatacttctgcgcaagatttgctatcaaaggtgattattgggggcagggaaccctggtaaccgtgagcagc

SEQ ID NO: 446 - protein, 116 aa, Artificial Sequence
  source: /note=“Description of Artificial Sequence: Synthetic polypeptide”
QVQLVQSGSELKKPGASVKVSCKASGYTFTNYGMNWVRQAPGQGLKWMGWINTYTGESTYADDFKGRFAFSLDTSASTAYLQISSLKAEDTAVYFCARFAIKGDYWGQGTLVTVSS

SEQ ID NO: 447 - DNA, 348 bp, Artificial Sequence
  source: /note=“Description of Artificial Sequence: Synthetic polynucleotide”
caggtccagctggtgcagtcaggttcagagctgaagaagcccggagcgtccgtcaaagtgtcatgcaaggcctcgggttacacgtttacgaactacggtatgaattgggtccgccaggctccgggccagggactgaaatggatgggatggatcaacacatatactggtgaatccacgtatgcggacgactttaaggggagattcgcgttcagccttgatacatcggcgtcgaccgcgtacctccaaatctcgtccttgaaagcagaggacactgcagtatacttttgtgcccggttcgctatcaagggagattattggggccaagggaccttggtgacagtgtccagc

SEQ ID NO: 448 - protein, 116 aa, Artificial Sequence
  source: /note=“Description of Artificial Sequence: Synthetic polypeptide”
QVQLVQSGAEVKKPGASVKISCKASGYTFTNYGMNWVRQAPGQGLKWMGWINTYTGESTYADDFKGRFTITLDTSASTAYMELSSLRSEDTAVYYCARFAIKGDYWGQGTLVTVSS

SEQ ID NO: 449 - DNA, 348 bp, Artificial Sequence
  source: /note=“Description of Artificial Sequence: Synthetic polynucleotide”
caagtccagttggtacaatcgggtgccgaggtaaagaaaccgggagcgtcggtcaaaatcagctgcaaggcctcaggctatacctttacaaactacggcatgaattgggtgagacaggcacccgggcagggattgaaatggatgggttggatcaacacgtatacaggggagtccacctatgcagatgactttaaggggcgcttcactatcacgctcgacacgtccgcgtcgacggcgtacatggaactgtcatcgcttcggagcgaagatacagccgtgtactattgtgctaggtttgcgattaagggagactactggggacagggaaccctcgtaactgtgtcatca

SEQ ID NO: 450 - protein, 116 aa, Artificial Sequence
  source: /note=“Description of Artificial Sequence: Synthetic polypeptide”
QVQLVQSGAEVKKPGASVKISCKASGYTFTNYGMNWVRQAPGQGLKWMGWINTYTGESTYADDFKGRFAFSLDTSASTAYLQLSSLRSEDTAVYFCARFAIKGDYWGQGTLVTVSS

SEQ ID NO: 451 - DNA, 348 bp, Artificial Sequence
  source: /note=“Description of Artificial Sequence: Synthetic polynucleotide”
caagtgcagctggtacaatcaggtgcggaggtgaaaaagccgggtgcctccgtcaagatttcatgcaaggcctcgggatacacattcaccaactatgggatgaattgggtgaggcaggctcccggtcaggggttgaaatggatggggtggattaacacatatacgggagaaagcacgtatgcggacgacttcaaggggcgcttcgcgttttccctggatacttcggcatcgactgcgtacctccagttgtcgtcgcttagatccgaggacacggccgtctacttctgtgcacggtttgcaatcaagggggattactggggacagggaacgcttgtaaccgtaagctca

SEQ ID NO: 452 - protein, 112 aa, Artificial Sequence
  source: /note=“Description of Artificial Sequence: Synthetic polypeptide”
DIVMTQSPLSLPVTPGEPASISCRSTKSLLHSNGITYLYWYLQKPGQSPQLLIYQMSNLASGVPDRFSGSGSGTDFTLKISRVEAEDVGVYYCAQNLEIPRTFGQGTKVEIK

SEQ ID NO: 453 - DNA, 663 bp, Artificial Sequence
  source: /note=“Description of Artificial Sequence: Synthetic polynucleotide”
gacattgtcatgacgcagtcgccgctctcgctcccggtcacaccgggagaacccgcgtccatttcatgcagatcgacaaagtcactcctccattcaaatggaatcacttacttgtactggtatcttcaaaaacccggtcagtcaccacagttgctcatctaccaaatgtccaatttggcttcgggagtgcccgaccgattcagcggttcggggagcggtacggattttacgttgaagatcagcagggtagaggcggaggacgtgggggtgtactattgtgcacagaaccttgaaattccacgcacctttggacaaggcaccaaggtcgaaatcaagcgtacggtggcggcaccttcagtgttcatctttcccccttccgatgaacagctcaaaagcgggactgcatccgtagtctgtcttttgaacaacttctatcccagagaggcgaaagtacagtggaaggtggacaacgcccttcaatcaggcaatagccaggagtcggtgacggagcaggattccaaagatagcacatactcgctttcatccactttgacattgtcgaaagcggactacgaaaagcacaaggtctatgcgtgcgaggtgacgcaccagggactttcgtcgccggtaaccaagtcgttcaatcgcggggagtgctgataa

SEQ ID NO: 454 - protein, 112 aa, Artificial Sequence
  source: /note=“Description of Artificial Sequence: Synthetic polypeptide”
DIVMTQSPSSLSASVGDKATITCRSTKSLLHSNGITYLYWYLQKPGKAPKLLIYQMSNLASGVPDRFSSSGSGTDFTLRISSVQPEDFATYYCAQNLEIPRTFGGGTKLEIK

SEQ ID NO: 455 - DNA, 336 bp, Artificial Sequence
  source: /note=“Description of Artificial Sequence: Synthetic polynucleotide”
gacattgtcatgacccagtcgccgtcatccctttcagcgagcgtgggggataaggcaacgatcacgtgtcgaagcaccaaatccttgctgcacagcaacgggattacgtatttgtactggtatttgcaaaaacccgggaaagccccgaagctcctcatctaccagatgtcgaatctggcgtcgggggtgccagatcggttctccagcagcgggtcgggtacagacttcacactccggatctcatcagtgcagcccgaggactttgcgacctactattgtgcccaaaatcttgaaatcccgagaacgttcggaggtggcacgaaattggagattaaa

SEQ ID NO: 456 - protein, 112 aa, Artificial Sequence
  source: /note=“Description of Artificial Sequence: Synthetic polypeptide”
DIVMTQSPLSLPVTPGEPASISCRSTKSLLHSNGITYLYWYLQKPGQSPQLLIYQMSNLASGVPDRFSSSGSGTDFTLKISRVEAEDVGVYYCAQNLEIPRTFGQGTKVEIK

SEQ ID NO: 457 - DNA, 336 bp, Artificial Sequence
  source: /note=“Description of Artificial Sequence: Synthetic polynucleotide”
gatatcgtaatgacccaatcgccgttgtcgcttccagtcacacccggggagcctgcttcgattagctgcagatcaacgaagtcgctcctccattcaaacgggattacgtatttgtactggtatcttcaaaagccgggtcagagcccgcagctgctgatctaccagatgtccaacttggcctcgggcgtccccgaccggtttagcagcagcgggtcgggaacggacttcactctcaagatctcaagggtcgaagcggaagatgtgggtgtgtattactgtgcgcagaatcttgagattccccgaacattcggtcagggaaccaaagtcgagatcaag

SEQ ID NO: 458 - protein, 112 aa, Artificial Sequence
  source: /note=“Description of Artificial Sequence: Synthetic polypeptide”
DIVMTQSPLSLPVTPGEPASISCRSTKSLLHSNGITYLYWYLQKPGQSPQLLIYQMSNLASGVPDRFSSSGSGTDFTLKISRVEAEDVGVYYCAQNLEIPRTFGGGTKLEIK

SEQ ID NO: 459 - DNA, 336 bp, Artificial Sequence
  source: /note=“Description of Artificial Sequence: Synthetic polynucleotide”
gacatcgtgatgacgcagtccccactgtcgctccctgtaacaccaggggagcccgcttccatttcgtgtaggtcaacgaagtccttgcttcatagcaatgggatcacttacttgtactggtatctccaaaaaccgggtcagtcccctcagttgctgatctaccagatgtcgaaccttgcgagcggtgtcccggatcgattttcatcgtccggatcgggaaccgacttcacactgaagattagccgcgtggaggccgaagatgtcggggtctactactgtgctcaaaaccttgagattccccggacgtttggaggcggcacgaagctggaaatcaag

SEQ ID NO: 460 - protein, 251 aa, Artificial Sequence
  source: /note=“Description of Artificial Sequence: Synthetic polypeptide”
QVQLVQSGSELKKPGESVKVSCKASGYTFTNYGMNWVRQAPGQCLKWMGWINTYTGESTYADDFKGRFAFSLDTSASTAYLQISSLKAEDTAVYFCARFAIKGDYWGQGTLVTVSSASTGGGGSGGGGSGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGCGTKVEIK

SEQ ID NO: 461 - DNA, 759 bp, Artificial Sequence
  source: /note=“Description of Artificial Sequence: Synthetic polynucleotide”
caggtacaactcgtgcagtcggggtcggagcttaagaagccgggagagtcggtaaaagtgtcgtgtaaagcctccgggtatacctttacgaattacggcatgaactgggtccggcaggcacccggtcagtgtctcaagtggatgggctggatcaatacgtacacgggcgaatccacttatgcagatgactttaagggaagattcgcgttctcattggacacatcagcgtcaacggcgtatcttcaaatttcgtccttgaaagcggaggacacggcggtctatttctgcgcacgattcgctatcaaaggagattactggggtcaggggacacttgtgacagtgtcgagcgccagcaccggaggtggagggagcggaggaggaggtagcggtggcggtggctcgggtggaggtggatcggacatcgtcatgactcagagccctctgtcactgcccgtaactcccggggagcccgcctccattagctgtcggtcgacaaagtccttgctccactcagatggaatcacgtatttgtactggtacttgcaaaagcctggacagtcgccacagctccttatctaccagttgtccaacctggcgtcgggagtaccggaccgcttttcatcatccgggtcaggcaccgatttcacactgaagattagccgcgtggaggcggaggacgaaggggtctactactgcgcccagaacctcgaaattccgaggacctttggatgcggtactaaagtggaaatcaagtgataa

SEQ ID NO: 462 - protein, 251 aa, Artificial Sequence
  source: /note=“Description of Artificial Sequence: Synthetic polypeptide”
QVQLVQSGSELKKPGESVKVSCKASGYTFTNYGMHWVRQAPGQGLKWMGWINTYTGESTYADDFKGRFAFSLDTSASTAYLQISSLKAEDTAVYFCARFAIKGDYWGQGTLVTVSSASTGGGGSGGGGSGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPYTFGQGTKVEIK

SEQ ID NO: 463 - DNA, 759 bp, Artificial Sequence
  source: /note=“Description of Artificial Sequence: Synthetic polynucleotide”
caggtacaactcgtgcagtcggggtcggagcttaagaagccgggagagtcggtaaaagtgtcgtgtaaagcctccgggtatacctttacgaattacggcatgcattgggtccggcaggcacccggtcaggggctcaagtggatgggctggatcaatacgtacacgggcgaatccacttatgcagatgactttaagggaagattcgcgttctcattggacacatcagcgtcaacggcgtatcttcaaatttcgtccttgaaagcggaggacacggcggtctatttctgcgcacgattcgctatcaaaggagattactggggtcaggggacacttgtgacagtgtcgagcgccagcaccggaggtggagggagcggaggaggaggtagcggtggcggtggctcgggtggaggtggatcggacatcgtcatgactcagagccctctgtcactgcccgtaactcccggggagcccgcctccattagctgtcggtcgacaaagtccttgctccactcagatggaatcacgtatttgtactggtacttgcaaaagcctggacagtcgccacagctccttatctaccagttgtccaacctggcgtcgggagtaccggaccgcttttcatcatccgggtcaggcaccgatttcacactgaagattagccgcgtggaggcggaggacgaaggggtctactactgcgcccagaacctcgaaattccgtacacctttggacaaggtactaaagtggaaatcaagtgataa

SEQ ID NO: 464 - protein, 251 aa, Artificial Sequence
  source: /note=“Description of Artificial Sequence: Synthetic polypeptide”
QVQLVQSGSELKKPGESVKVSCKASGYTFTNYGMNWVRQAPGQGLKWMGWINTYTGESTYADDFKGRFAFSLDTSASTAYLQISSLKAEDTAVYFCARFAIKGDYWGQGTLVTVSSASTGGGGSGGGGSGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGQGTKVEIK

SEQ ID NO: 465 - DNA, 759 bp, Artificial Sequence
  source: /note=“Description of Artificial Sequence: Synthetic polynucleotide”
caggtacaactcgtgcagtcggggtcggagcttaagaagccgggagagtcggtaaaagtgtcgtgtaaagcctccgggtatacctttacgaattacggcatgaactgggtccggcaggcacccggtcaggggctcaagtggatgggctggatcaatacgtacacgggcgaatccacttatgcagatgactttaagggaagattcgcgttctcattggacacatcagcgtcaacggcgtatcttcaaatttcgtccttgaaagcggaggacacggcggtctatttctgcgcacgattcgctatcaaaggagattactggggtcaggggacacttgtgacagtgtcgagcgccagcaccggaggtggagggagcggaggaggaggtagcggtggcggtggctcgggtggaggtggatcggacatcgtcatgactcagagccctctgtcactgcccgtaactcccggggagcccgcctccattagctgtcggtcgacaaagtccttgctccactcagatggaatcacgtatttgtactggtacttgcaaaagcctggacagtcgccacagctccttatctaccagttgtccaacctggcgtcgggagtaccggaccgcttttcatcatccgggtcaggcaccgatttcacactgaagattagccgcgtggaggcggaggacgaaggggtctactactgcgcccagaacctcgaaattccgaggacctttggacaaggtactaaagtggaaatcaagtgataa

SEQ ID NO: 466 - protein, 251 aa, Artificial Sequence
  source: /note=“Description of Artificial Sequence: Synthetic polypeptide”
DIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGQGTKVEIKRTGGGGSGGGGSGGGGSGGGGSGQVQLVQSGSELKKPGESVKVSCKASGYTFTNYGMNWVRQAPGQGLKWMGWINTYTGESTYADDFKGRFAFSLDTSASTAYLQISSLKAEDTAVYFCARFAIKGDYWGQGTLVTVSS

SEQ ID NO: 467 - DNA, 759 bp, Artificial Sequence
  source: /note=“Description of Artificial Sequence: Synthetic polynucleotide”
gatatcgtgatgacacagtcacccctgtcgctccctgtgactcccggagagcctgcgtccatctcgtgccggtcaacaaagtccctgctgcacagcgacggaattacgtatttgtactggtatttgcaaaaacccggacagagcccgcaattgttgatctaccagctctcaaaccttgcgagcggggtcccggatcgctttagctcgtcggggtcaggaactgacttcacactcaaaatctcaagggtcgaggccgaggatgaaggtgtctattactgcgctcagaatctcgaaatcccgcggacctttggtcagggtacgaaggtagagatcaagcgaacgggaggcggtgggtccggtggcggtggttccggcggaggtggttcgggaggaggaggtagcggtcaggtgcagcttgtccagtcgggatcggagctcaagaagccaggagagtcggtgaaagtatcgtgtaaagcctcggggtacacattcacgaactacggcatgaattgggtgagacaagctcccgggcagggcttgaaatggatgggatggattaacacctacacaggggaatccacatatgcggacgacttcaaggggaggttcgcgttttcacttgatacttcagcgagcacggcgtatctccaaatttcgtcgcttaaggcagaagataccgcagtatacttttgcgccagattcgcgattaaaggggactattggggacaggggacccttgtcacggtgtcatcatgataa

SEQ ID NO: 468 - protein, 250 aa, Artificial Sequence
  source: /note=“Description of Artificial Sequence: Synthetic polypeptide”
DIVMTQSPLSLPVTPGEPASISCRSTKSLLHSNGITYLYWYLQKPGQSPQLLIYQMSNLASGVPDRFSSSGSGTDFTLKISRVEAEDEGVYYCAQNLEIPRTFGGGTKLEIKRTGGGGSGGGGSGGGGSGGGGSQVQLVQSGAEVKKPGESVKISCKASGYTFTNYGMNWVRQAPGQGLKWMGWINTYTGESTYADDFKGRFAFSLDTSASTAYLQLSSLRSEDTAVYFCARFAIKGDYWGQGTLVTVSS

SEQ ID NO: 469 - DNA, 756 bp, Artificial Sequence
  source: /note=“Description of Artificial Sequence: Synthetic polynucleotide”
gatatcgtgatgacacagtcacccctgtcgctccctgtgactcccggagagcctgcgtccatctcgtgccggtcaacaaagtccctgctgcacagcaatggaattacgtatttgtactggtatttgcaaaaacccggacagagcccgcaattgttgatctaccagatgtcaaaccttgcgagcggggtcccggatcgctttagctcgtcggggtcaggaactgacttcacactcaaaatctcaagggtcgaggccgaggatgaaggtgtctattactgcgctcagaatctcgaaatcccgcggacctttggtggaggtacgaagctggagatcaagcgaacgggaggcggtgggtccggtggcggtggttccggcggaggtggttcgggaggaggaggtagccaggtgcagcttgtccagtcgggagcagaggtaaagaagccaggagagtcggtgaaaatctcgtgtaaagcctcggggtacacattcacgaactacggcatgaattgggtgagacaagctcccgggcagggcttgaaatggatgggatggattaacacctacacaggggaatccacatatgcggacgacttcaaggggaggttcgcgttttcacttgatacttcagcgagcacggcgtatctccaactctcgtcgcttcgctccgaagataccgcagtatacttttgcgccagattcgcgattaaaggggactattggggacaggggacccttgtcacggtgtcatcatgataa

SEQ ID NO: 470 - protein, 250 aa, Artificial Sequence
  source: /note=“Description of Artificial Sequence: Synthetic polypeptide”
DIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGGGTKLEIKRTGGGGSGGGGSGGGGSGGGGSQVQLVQSGAEVKKPGESVKISCKASGYTFTNYGMNWVRQAPGQGLKWMGWINTYTGESTYADDFKGRFAFSLDTSASTAYLQLSSLRSEDTAVYFCARFAIKGDYWGQGTLVTVSS

SEQ ID NO: 471 - DNA, 756 bp, Artificial Sequence
  source: /note=“Description of Artificial Sequence: Synthetic polynucleotide”
gatatcgtgatgacacagtcacccctgtcgctccctgtgactcccggagagcctgcgtccatctcgtgccggtcaacaaagtccctgctgcacagcgacggaattacgtatttgtactggtatttgcaaaaacccggacagagcccgcaattgttgatctaccagctctcaaaccttgcgagcggggtcccggatcgctttagctcgtcggggtcaggaactgacttcacactcaaaatctcaagggtcgaggccgaggatgaaggtgtctattactgcgctcagaatctcgaaatcccgcggacctttggtggaggtacgaagctggagatcaagcgaacgggaggcggtgggtccggtggcggtggttccggcggaggtggttcgggaggaggaggtagccaggtgcagcttgtccagtcgggagcagaggtaaagaagccaggagagtcggtgaaaatctcgtgtaaagcctcggggtacacattcacgaactacggcatgaattgggtgagacaagctcccgggcagggcttgaaatggatgggatggattaacacctacacaggggaatccacatatgcggacgacttcaaggggaggttcgcgttttcacttgatacttcagcgagcacggcgtatctccaactgtcgtcgcttcgctccgaagataccgcagtatacttttgcgccagattcgcgattaaaggggactattggggacaggggacccttgtcacggtgtcatcatgataa

SEQ ID NO: 472 - protein, 250 aa, Artificial Sequence
  source: /note=“Description of Artificial Sequence: Synthetic polypeptide”
DIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGCGTKLEIKRTGGGGSGGGGSGGGGSGGGGSQVQLVQSGAEVKKPGESVKISCKASGYTFTNYGMNWVRQAPGQCLKWMGWINTYTGESTYADDFKGRFAFSLDTSASTAYLQLSSLRSEDTAVYFCARFAIKGDYWGQGTLVTVSS

SEQ ID NO: 473 - DNA, 756 bp, Artificial Sequence
  source: /note=“Description of Artificial Sequence: Synthetic polynucleotide”
gatatcgtgatgacacagtcacccctgtcgctccctgtgactcccggagagcctgcgtccatctcgtgccggtcaacaaagtccctgctgcacagcgacggaattacgtatttgtactggtatttgcaaaaacccggacagagcccgcaattgttgatctaccagctctcaaaccttgcgagcggggtcccggatcgctttagctcgtcggggtcaggaactgacttcacactcaaaatctcaagggtcgaggccgaggatgaaggtgtctattactgcgctcagaatctcgaaatcccgcggacctttggttgcggtacgaagctggagatcaagcgaacgggaggcggtgggtccggtggcggtggttccggcggaggtggttcgggaggaggaggtagccaggtgcagcttgtccagtcgggagcagaggtaaagaagccaggagagtcggtgaaaatctcgtgtaaagcctcggggtacacattcacgaactacggcatgaattgggtgagacaagctcccgggcagtgtttgaaatggatgggatggattaacacctacacaggggaatccacatatgcggacgacttcaaggggaggttcgcgttttcacttgatacttcagcgagcacggcgtatctccaactgtcgtcgcttcgctccgaagataccgcagtatacttttgcgccagattcgcgattaaaggggactattggggacaggggacccttgtcacggtgtcatcatgataa

SEQ ID NO: 474 - protein, 253 aa, Artificial Sequence
  source: /note=“Description of Artificial Sequence: Synthetic polypeptide”
QVQLVQSGAEVKKPGESVKISCKASGYTFTNYGMNWVRQAPGQGLKWMGWINTYTGESTYADDFKGRFAFSLDTSASTAYLQLSSLRSEDTAVYFCARFAIKGDYWGQGTLVTVSSASTGGGGSGGGGSGGGGSGGGGSDIVMTQSPLSLPVTPGEPASISCRSTKSLLHSDGITYLYWYLQKPGQSPQLLIYQMSNLASGVPDRFSSSGSGTDFTLKISRVEAEDEGVYYCAQNLEIPRTFGGGTKLEIKRT

SEQ ID NO: 475 - DNA, 765 bp, Artificial Sequence
  source: /note=“Description of Artificial Sequence: Synthetic polynucleotide”
caggtgcagctcgtccagtcaggggcggaggtcaagaaaccaggagagtcagtaaagatctcgtgcaaagcgtcgggatatacctttacaaactacggcatgaattgggtgcgacaagcacccggccagggcctgaagtggatggggtggatcaatacatatactggggagtccacttatgccgacgacttcaagggaaggtttgccttctccctggatacgtcggcgtcgaccgcttatttgcagttgagctcgctgaggtcggaagatacagcagtgtacttctgcgctcgcttcgcaatcaaaggggattactggggtcaggggacgcttgtaaccgtgtcctcagcgtcgacgggtggtggtgggtcgggaggtggtggtagcggaggtggagggtcgggtggaggcggatcagatattgtgatgacacaatcgccgctctcactgcccgtaacgcccggagagcccgcgtcaatttcatgtcggtcgacaaagtcactccttcactcggacgggattacgtacctctattggtatcttcaaaagccgggtcagtcacctcaactcctcatctaccagatgtcgaacttggcatcaggggtccctgatcgcttctcgtccagcgggtccgggacggactttaccttgaaaatctcaagagtggaggccgaggacgaaggggtctactactgtgcccagaaccttgaaattccgcggacgtttggaggagggacaaagctggagatcaaaagaacttgataa

SEQ ID NO: 476 - protein, 252 aa, Artificial Sequence
  source: /note=“Description of Artificial Sequence: Synthetic polypeptide”
QVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSSASTGGGGSGGGGSGGGGSGGGGSGDIVMTQSPSSLSASVGDKATITCRSTKSLLHSNGITYLYWYLQKPGKAPKLLIYQMSNLASGVPDRFSSSGSGTEFTLTISSVQPEDEGTYYCAQNLEIPRTFGQGTKLEIK

SEQ ID NO: 477 - DNA, 762 bp, Artificial Sequence
  source: /note=“Description of Artificial Sequence: Synthetic polynucleotide”
caggtacaactccagcaatcaggaggcgggcttgtaaaacccggggagtcggtgaagatcagctgtgcagcatcagggtacacattcacaaactatggcatgaactgggtcaaacaggcgccagggaaggggctgaaatggatgggctggatcaatacgtacactggtgagagcacatacgcggatgatttcaaagggcggtttacgttttcactcgaaacgtcggcgtccgccgcatatcttcaaatcaattccttgaggccggaggacacagcagtctacttctgcgcccgatttgccattaagggtgattattggggccagggaaccacggtgactgtcagctccgcttcgacaggaggaggagggagcggtggaggaggatcgggaggtggagggtcgggtggtggtgggtcaggagacattgtgatgacccagtcgccctcgtcgctttcagcgagcgtcggggacaaggcgaccattacttgtcgctcaactaagtcgttgctgcactccaacgggatcacgtacctctattggtatctccaaaaacctggtaaagcgcctaagctcctgatctaccagatgtccaatttggcgtcgggagtaccggacagattttcaagctccggatcagggactgagttcacacttacgattagctccgtgcagcccgaggatgaagggacctattactgcgcccagaacttggaaatcccgagaaccttcggacagggtacgaagctggaaatcaagtgataa

SEQ ID NO: 478 - protein, 252 aa, Artificial Sequence
  source: /note=“Description of Artificial Sequence: Synthetic polypeptide”
QVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSSASTGGGGSGGGGSGGGGSGGGGSGDIVMTQSPSSLSASVGDKATITCRSTKSLLHSDGITYLYWYLQKPGKAPKLLIYQLSNLASGVPDRFSSSGSGTEFTLTISSVQPEDEGTYYCAQNLEIPRTFGQGTKLEIK

SEQ ID NO: 479 - DNA, 762 bp, Artificial Sequence
  source: /note=“Description of Artificial Sequence: Synthetic polynucleotide”
caggtccagttgcaacagtccggaggtgggcttgtgaaacccggggagtcggtgaagatctcatgcgcggcttcggggtacacgttcacaaactacgggatgaattgggtcaagcaggcacccgggaaggggttgaaatggatgggttggatcaatacatacactggggagagcacgtatgcagatgacttcaaaggacgctttaccttcagcctcgaaacgagcgcctcagccgcatacctccaaatcaatagcctccggccagaagatactgcggtgtatttctgcgctaggttcgccattaaaggggactattggggacaggggacgacggtgacggtatcatcagcgtcgactggtggaggcggctccggaggaggtgggtccggcggtggcgggtcaggtggtggtggctcgggagatattgtaatgacacagtccccgtcctccctctccgcgagcgtgggagataaggccacgattacatgtcgaagcaccaaatcgctccttcactcggacggaatcacatatttgtattggtacttgcaaaaaccggggaaggcgcctaagctgctgatctaccagctttcgaacctggcgtcaggggtccctgaccggttttcgtcgtcgggaagcgggaccgagtttacacttacgatctcgtcagtacagcccgaggacgaaggaacatactattgcgcccagaacttggagattccgagaacttttggccagggaaccaagctcgaaatcaagtgataa

SEQ ID NO: 480 - protein, 251 aa, Artificial Sequence
  source: /note=“Description of Artificial Sequence: Synthetic polypeptide”
DIVMTQSPSSLSASVGDKATITCRSTKSLLHSNGITYLYWYLQKPGKAPKLLIYQMSNLASGVPDRFSSSGSGTEFTLTISSVQPEDEGTYYCAQNLEIPRTFGQGTKLEIKRTGGGGSGGGGSGGGGSGGGGSGQVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSS

SEQ ID NO: 481 - DNA, 759 bp, Artificial Sequence
  source: /note=“Description of Artificial Sequence: Synthetic polynucleotide”
gacatcgtcatgacccagtcaccgtcctcactgtcggcgtcggtgggtgataaggccacaattacatgccgcagcacgaaatcactgctccactccaatgggattacatatctctattggtatctccaaaaacccggaaaggcacctaagttgctgatctaccagatgtcgaacttggcatcgggagtacccgataggttctcgtcgtcgggaagcggcacggagttcacgctcaccatttcctcagtccagccggaggacgaaggaacttactactgcgctcagaatcttgaaatcccgcgcacatttggacaagggacgaaacttgaaatcaagcgaactggaggaggtgggtcaggcggaggtgggagcggcggaggcggatcgggtggtggagggtcgggacaggtacagttgcagcagtcgggaggcgggctggtaaaacctggtgaaagcgtcaagatctcatgtgcagcctcagggtatacgttcaccaattacgggatgaactgggtgaagcaggcgccagggaaaggtcttaagtggatgggatggatcaacacttacacgggagagtccacatacgcggatgactttaaggggcggttcacgttttcgttggagacttcagcgtccgctgcctacctccaaatcaactcccttagacccgaggacacagcggtctatttctgtgcgcggtttgccattaaaggtgattattggggacagggtacgactgtgaccgtgtccagctgataa

SEQ ID NO: 482 - protein, 251 aa, Artificial Sequence
  source: /note=“Description of Artificial Sequence: Synthetic polypeptide”
DIVMTQSPSSLSASVGDKATITCRSTKSLLHSDGITYLYWYLQKPGKAPKLLIYQLSNLASGVPDRFSSSGSGTEFTLTISSVQPEDEGTYYCAQNLEIPRTFGQGTKLEIKRTGGGGSGGGGSGGGGSGGGGSGQVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSS

SEQ ID NO: 483 - DNA, 759 bp, Artificial Sequence
  source: /note=“Description of Artificial Sequence: Synthetic polynucleotide”
gacatcgtcatgacccagtcaccgtcctcactgtcggcgtcggtgggtgataaggccacaattacatgccgcagcacgaaatcactgctccactccgacgggattacatatctctattggtatctccaaaaacccggaaaggcacctaagttgctgatctaccagttgtcgaacttggcatcgggagtacccgataggttctcgtcgtcgggaagcggcacggagttcacgctcaccatttcctcagtccagccggaggacgaaggaacttactactgcgctcagaatcttgaaatcccgcgcacatttggacaagggacgaaacttgaaatcaagcgaactggaggaggtgggtcaggcggaggtgggagcggcggaggcggatcgggtggtggagggtcgggacaggtacagttgcagcagtcgggaggcgggctggtaaaacctggtgaaagcgtcaagatctcatgtgcagcctcagggtatacgttcaccaattacgggatgaactgggtgaagcaggcgccagggaaaggtcttaagtggatgggatggatcaacacttacacgggagagtccacatacgcggatgactttaaggggcggttcacgttttcgttggagacttcagcgtccgctgcctacctccaaatcaactcccttagacccgaggacacagcggtctatttctgtgcgcggtttgccattaaaggtgattattggggacagggtacgactgtgaccgtgtccagctgataa

SEQ ID NO: 484 - protein, 253 aa, Artificial Sequence
  source: /note=“Description of Artificial Sequence: Synthetic polypeptide”
DIVMTQSPSSLSASVGDKATITCRSTKSLLHSNGITYLYWYLQKPGKAPKLLIYQMSNLASGVPDRFSSSGSGTEFTLTISSVQPEDEGTYYCAQNLEIPRTFGQGTKLEIKRTPSHNSHQVPSAGGPTANSGTSGSQVQLQQSGGGLVKPGESVKISCAASGYTFTNYGMNWVKQAPGKGLKWMGWINTYTGESTYADDFKGRFTFSLETSASAAYLQINSLRPEDTAVYFCARFAIKGDYWGQGTTVTVSS

SEQ ID NO: 485 - DNA, 765 bp, Artificial Sequence
  source: /note=“Description of Artificial Sequence: Synthetic polynucleotide”
gacatcgtcatgacccagtcaccgtcctcactgtcggcgtcggtgggtgataaggccacaattacatgccgcagcacgaaatcactgctccactccaatgggattacatatctctattggtatctccaaaaacccggaaaggcacctaagttgctgatctaccagatgtcgaacttggcatcgggagtacccgataggttctcgtcgtcgggaagcggcacggagttcacgctcaccatttcctcagtccagccggaggacgaaggaacttactactgcgctcagaatcttgaaatcccgcgcacatttggacaagggacgaaacttgaaatcaagcgaactccgtcccacaacagccatcaagtgccctcggcgggagggcccaccgccaattcggggacatcagggagccaggtacagttgcagcagtcgggaggcgggctggtaaaacctggtgaaagcgtcaagatctcatgtgcagcctcagggtatacgttcaccaattacgggatgaactgggtgaagcaggcgccagggaaaggtcttaagtggatgggatggatcaacacttacacgggagagtccacatacgcggatgactttaaggggcggttcacgttttcgttggagacttcagcgtccgctgcctacctccaaatcaactcccttagacccgaggacacagcggtctatttctgtgcgcggtttgccattaaaggtgattattggggacagggtacgactgtgaccgtgtccagctgataa

SEQ ID NO: 486 - protein, 112 aa, Artificial Sequence
  source: /note=“Description of Artificial Sequence: Synthetic polypeptide”
DIVMTQSAFSNPVTLGTSASISCRSTKSLLHSNGITYLYWYLQKPGQSPQLLIYQMSNLASGVPDRFSSSGSGTDFTLRISRVEAEDVGVYYCAQNLEIPRTFGGGTKLEIK

SEQ ID NO: 487 - protein, 116 aa, Artificial Sequence
  source: /note=“Description of Artificial Sequence: Synthetic polypeptide”
QVQLQQSGPELKKPGETVKISCKASGYTFTNYGMNWVKQAPGRGLKWMGWINTYTGESTYADDFKGRFAFSLETSASAAYLQINNLKNEDTATYFCARFAIKGDYWGQGTTLTVSS

SEQ ID NO: 488 - protein, 253 aa, Artificial Sequence
  source: /note=“Description of Artificial Sequence: Synthetic polypeptide”
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRTPSHNSHQVPSAGGPTANSGTSGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSS

SEQ ID NO: 489 - protein, 1105 aa, Artificial Sequence
  source: /note=“Description of Artificial Sequence: Synthetic polypeptide”
QLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPSCPAPEFLGGPSVFLFPPKPKDTLMISRTPEVTCVVVDVSQEDPEVQFNWYVDGVEVHNAKTKPREEQFNSKYRVVSVLTVLHQDWLNGKEYKCKVSNKGLPSSIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSKSCDKTGGGGSGGGGSGGGGSGGGGSGGGGSGGGGSGGGGSGGGGSCPSCPAPEFLGGPSVFLFPPKPKDTLMISRTPEVTCVVVDVSQEDPEVQFNWYVDGVEVHNAKTKPREEQFNSDYRVVSVLTVLHQDWLNGKEYKCKVSNKGLPSSIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSGECGGGGSGGGGSDIVMTQSPLSLPVTPGEPASISCRSTKSLLHSDGITYLYWYLQKPGQSPQLLIYQLSNLASGVPDRFSSSGSGTDFTLKISRVEAEDEGVYYCAQNLEIPRTFGCGTKLEIKRTGGGGSGGGGSGGGGSDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRTPSHNSHQVPSAGGPTANSGTSGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSS

(19) United States
(12) Patent Application Publication
(10) Pub. No.: US 2017/0306415 A1
(43) Pub. Date: Oct. 26, 2017

(54) PREDICTING TUMOR RESPONSES TO ANTIBODIES AGAINST HEPATOCYTE GROWTH FACTOR (HGF) AND/OR ITS COGNATE RECEPTOR, C-MET

(71) Applicant: Merrimack Pharmaceuticals, Inc., Cambridge, MA (US)

(72) Inventors: Adnan ABU-YOUSIF, Boston, MA (US); Aaron FULGHAM, Belmont, MA (US); Brian HARMS, Roslindale, MA (US); Gavin MACBEATH, Wakefield, MA (US); Victoria RIMKUNAS, Reading, MA (US)

(73) Assignee: Merrimack Phamaceuticals, Inc., Cambridge, MA (US)

(21) Appl. No.: 15/515,889

(22) PCT Filed: Oct. 1, 2015
(86) PCT No.: PCT/US15/53561

§ 371 Date: Mar. 30, 2017

Related U.S. Application Data
(60) Provisional application No. 62/217,441, filed on Sep. 11, 2015.
(60) Provisional application No. 62/058,473, filed on Oct. 1, 2014.

(51) Int. Cl. C12Q 1/68; C07K 16/28; G01N 33/574; C07K 16/30; G06F 19/00

(52) U.S. Cl. CPC C12Q 1/6886; G01N 33/574; C07K 16/30; C07K 16/2863; G01N 33/57492; C12Q 2600/106; C07K 2317/31; C12Q 2600/158; G01N 2333/4753; G01N 2333/705; G01N 2333/71; G06F 19/3431

(57) ABSTRACT

The present invention provides methods for selecting a therapy for, or for providing a treatment to, a patient having a cancer. The methods of the present invention comprise detecting the protein biomarkers c-Met, HGF, and EpCAM in cancer cells or cancerous tissues of a subject, and predicting the subject's responsiveness to treatment with a c-Met-targeted or other HGF-inhibitory treatment, thereby enabling the selection and administration of an effective therapeutic.

### REFERENCE TO RELATED APPLICATIONS

This application claims priority to U.S. Provisional Application Ser. No. 62/217,441, filed Sep. 11, 2015 and U.S. Provisional Application Ser. No. 62/058,473, filed Oct. 1, 2014, each of which is incorporated herein by reference in its entirety.

### BACKGROUND

Targeted biologic agents are attractive therapeutic modalities because of the exquisite specificity they have for specific antigens. In malignant lesions, antigen expression is frequently upregulated and/or the associated pathway deregulated. The use of biomarkers for predicting outcomes to therapies has increased with the development of more targeted, specific therapeutics. For example, Trastuzumab (Herceptin®) is a monoclonal antibody that targets the HER2/neu receptor on cancer cells. The effect of trastuzumab is most pronounced when assays to detect Her-2/ErbB2 protein expression are used to determine if a patient is likely to benefit from such treatment. The predictive power of this assay has ensured that the test is now commonplace for diagnosis of breast cancers. Interestingly, efforts to employ a similar approach do not always confer the same predictive power in the context of other therapeutic targets. For example, response to the anti-Epidermal growth factor receptor (EGFR) therapy cetuximab (Erbitux®) in patients whose tumors express EGFR is only 15-20%. In fact, the most predictive tool for EGFR therapies is determination of the mutation status of KRAS.

Additional receptor tyrosine kinases outside of the EGFR subfamily have also been identified as drivers of cancer cell survival and spread. The hepatocyte growth factor (HGF)/c-Met signaling axis is one such example, which when aberrantly activated, is associated with cell proliferation, angiogenesis, invasion and metastasis of different tumors. Pathway activation can occur through protein overexpression, mutation, gene amplification, and also paracrine or autocrine up-regulation of the ligand, HGF. Accordingly, anti-Met inhibitors, including anti-Met monoclonal antibodies, are in development for use in the treatment of various cancers.

No biologic agents targeting HGF or c-Met have received regulatory approval to date. Approval of an agent that targets the HGF/c-Met signaling pathway may require molecules that more completely inhibit the pathway and do not activate the pathway. Such molecules include bispecific anti-c-Met, anti-EpCAM antibodies such as Ab#5, Ab#7, and Ab#13, each of which is disclosed in copending U.S. patent application Ser. No. 14/199,760, and in PCT publication No. WO/2014/138449. In addition, there is a need to develop and implement diagnostic and theranostic strategies that help identify the patients most likely to benefit from treatment. Disclosed herein are biomarker criteria and methodology to detect key mediators for anti-Met therapies in order to determine the optimum treatment strategy.

### SUMMARY

Provided herein are methods suitable for a) predicting responsiveness to, or b) selecting for treatment with a c-Met-targeted or other HGF-inhibitory treatment. These methods involve detection and quantification of protein biomarkers c-Met, HGF and EpCAM in cancer cells (including associated stromal cells in cancerous tumors). Suitable cancer cell samples include, but are not limited to, cells obtained by surgical tissue resection or core or fine needle biopsy (e.g., from tumors), and circulating tumor cells. Each of the biomarkers may be detected and measured as the protein, and may also or alternatively be detected and measured as an RNA (e.g., a gene transcript) that encodes the protein, e.g., by detecting and quantifying RNA in cancer cells that specifically hybridizes with sequences complementary to sequences encoding the protein. These levels are measured in at least one cancer cell sample obtained from the patient.

Accordingly, in one aspect a method is provided for selecting therapy for, or for providing treatment to, a patient having a cancer, the method comprising obtaining at least one biomarker score from at least one cancer cell sample from patient, wherein the at least one biomarker score comprises a score for one of c-Met and HGF; and, if each score meets a threshold, then 1) selecting the patient for treatment with, and/or 2) administering to the patient, an effective amount of a bispecific anti-EpCAM/anti-c-Met antibody, wherein the threshold comprises only one of:
i. an immunohistochemistry (IHC) score for c-Met of 2+ or higher; or
ii. a c-Met IHC score of 3+ or higher; or
iii. an HGF RNA in situ hybridization (ISH) score of 1+ or higher; or
iv. an HGF RNA ISH score of 2+ or higher; or
v. an HGF RNA ISH score of 3+ or higher; or
vi. an HGF RNA ISH score of 4+ or higher, an HGF
vii. an HGF IHC score of 1+ or higher;
viii. an HGF IHC score of 2+ or higher;
ix. an HGF IHC score of 3+ or higher; or
x. an HGF IHC score of 4+.

In one embodiment, the threshold is a c-Met IHC score of 3+. In another embodiment, the threshold is a c-Met IHC score of 2+ or higher. In yet another embodiment the threshold is an HGF (by RNA ISH or IHC) score of 1+ or higher.

In another aspect a method is provided for selecting therapy for, or for providing treatment to, a patient having a cancer, the method comprising obtaining at least two biomarker scores from at least one biopsy sample of the cancer from the patient, wherein the scored biomarkers comprise c-Met and HGF; and, if the scores meet a threshold, then 1) selecting the patient for treatment with, or 2) administering to the patient, an effective amount of a bispecific anti-EpCAM/anti-c-Met antibody, wherein the threshold comprises only one of:
i. an HGF (by RNA ISH or IHC) score of 1+ or higher and a c-Met IHC score of 1+ or higher; or
ii. an HGF RNA ISH or IHC score of 1+ or higher and a c-Met IHC score of 2+ or higher; or
iii. an HGF RNA ISH or IHC score of 1+ or higher and a c-Met IHC score of 3+; or
iv. an HGF RNA ISH or IHC score of 2+ or higher and a c-Met IHC score of 1+ or higher; or
v. an HGF RNA ISH or IHC score of 2+ or higher and a c-Met IHC score of 2+ or higher; or
vi. an HGF RNA ISH or IHC score of 2+ or higher and a c-Met IHC score of 3+; or
vii. an HGF RNA ISH or IHC score of 3+ or higher and a c-Met IHC score of 1+ or higher; or
viii. an HGF RNA ISH or IHC score of 3+ or higher and a c-Met IHC score of 2+ or higher; or
ix. an HGF RNA ISH or IHC score of 3+ or higher and a c-Met IHC score of 3+; or
x. an HGF RNA ISH or IHC score of 4+ and a c-Met IHC score of 1+ or higher; or
xi. an HGF RNA ISH or IHC score of 4+ and a c-Met IHC score of 2+ or higher; or
xii. an HGF RNA ISH or IHC score of 4+ and a c-Met IHC score of 3+; or
xiii. an HGF RNA ISH or IHC score of 0 and a c-Met IHC score of 2+; or
xiv. an HGF RNA ISH or IHC score of 0 and a c-Met IHC score of 3+.

In an embodiment of either of the preceding aspects, the method further comprises obtaining an EpCAM IHC biomarker score from the biopsy sample (e.g., from cancer cells within the sample), wherein the threshold further comprises an EpCAM IHC score of 1+ or higher (or 2+ or higher, or 3+).

In an embodiment, a bispecific anti-EpCAM/anti-c-Met antibody is used in treating cancer in a patient, wherein at least one cancer cell sample from the patient has at least one biomarker score for one of c-met and HGF, and each score meets a threshold, wherein the threshold comprises only one of (i)-(x). In further embodiments, the patient has been tested and found to have at least one biomarker score from at least one cancer cell sample from the patient for one of c-Met and HGF that meets the threshold.

In an embodiment, a bispecific anti-EpCAM/anti-c-Met antibody is used in treating cancer in a patient, wherein at least one cancer cell sample from the patient has at least two biomarker scores, wherein the scored biomarkers comprise c-met and HGF and the scores meet a threshold, wherein the threshold comprises only one of (i)-(xiv). In further embodiments, the patient has been tested and found to have at least two biomarker scores from at least one cancer cell sample from the patient for c-met and HGF that meets the threshold.

In an embodiment, a bispecific anti-EpCAM/anti-c-Met antibody is used in treating cancer in a patient wherein the treatment comprises testing a cancer cell sample from the patient to determine whether at least one more biomarker score for one of c-Met and HGF meets a threshold, wherein the threshold comprises only one of (i)-(x) and beginning administration of the drug if the threshold is met.

In yet another embodiment, a bispecific anti-EpCAM/anti-c-Met antibody is used in treating cancer in a patient wherein the treatment comprises testing a cancer cell sample from the patient to determine whether at least two biomarker scores for the biomarkers c-Met and HGF meets a threshold, wherein the threshold comprises only one of (i)-(xiv) and beginning administration of the drug if the threshold is met.

In an embodiment, methods of testing for responsiveness of, or selecting therapy for, a cancer patient to treatment with a bispecific anti-EpCAM/anti-c-Met antibody are provided, the methods comprising testing at least one cancer cell sample from said patient for at least one biomarker selected from c-Met and HGF and wherein if each score meets a threshold, the patient is responsive to said antibody (or selected for treatment with said antibody, wherein the threshold comprises only one of (i)-(x) (e.g. where the cancer cell sample is tested in vitro).

In an embodiment, methods of testing for responsiveness of, or selecting therapy for, a cancer patient to treatment with a bispecific anti-EpCAM/anti-c-Met antibody are provided, the methods comprising testing at least one cancer cell sample from said patient for at least two biomarkers selected from c-Met and HGF, and wherein if the scores meet a threshold, the patient is responsive to said antibody (or selected for treatment with said antibody), wherein the threshold comprises only one of (i)-(xiv) (e.g. where the cancer cell sample is tested in vitro).

In an embodiment, at least one biomarker selected from c-Met and HGF is used for assessing responsiveness of, or selecting therapy for, a cancer patient to treatment with a bispecific anti-EpCAM/anti-c-Met antibody (e.g. where the cancer cell sample is tested in vitro).

In an embodiment, at least two biomarkers selected from c-Met and HGF are used for assessing responsiveness of, or selecting therapy for, a cancer patient to treatment with a bispecific anti-EpCAM/anti-c-Met antibody (e.g. where the cancer cell sample is tested in vitro).

In an embodiment, a probe or an antibody for at least one biomarker selected from c-Met and HGF is used for assessing responsiveness of a cancer patient to treatment with a bispecific anti-EpCAM/anti-c-Met antibody (e.g. where a cancer cell sample from the patient is tested in vitro for the biomarker).

In an embodiment, at least one biomarker selected from c-Met and HGF is used in treating cancer in a patient wherein the treatment comprises obtaining at least one biomarker score from at least one cancer cell sample from the patient, wherein the at least one biomarker score comprises a score for one of c-Met and HGF; and if each score meets a threshold, administering an effective amount of a bispecific anti-EpCAM/anti-c-Met antibody, wherein the threshold comprises only one of (i)-(x).

In an embodiment, at least one biomarker selected from c-Met and HGF is used in treating cancer in a patient wherein the treatment comprises obtaining at least two biomarker scores from at least one cancer cell sample from the patient, wherein the scored biomarkers comprise c-Met and HGF; and if the scores meet a threshold, administering an effective amount of a bispecific anti-EpCAM/anti-c-Met antibody, wherein the threshold comprises only one of (i)-(xiv).

In an embodiment, a probe or an antibody for at least one biomarker selected from c-Met and HGF is used in treating cancer in a patient wherein the treatment comprises using the probe or antibody to obtain at least one biomarker score from at least one cancer cell sample from the patient wherein the at least one biomarker score comprises a score for one of c-Met and HGF; and if each score meets a threshold, administering an effective amount of a bispecific anti-EpCAM/anti-c-Met antibody, wherein the threshold comprises only one of (i)-(x).

In an embodiment, a probe or an antibody for at least one biomarker selected from c-Met and HGF is used in treating cancer in a patient wherein the treatment comprises using the probe or antibody to obtain at least two biomarker scores from at least one cancer cell sample from the patient, wherein the scored biomarkers comprise c-Met and HGF; and if the scores meet a threshold, administering an effective amount of a bispecific anti-EpCAM/anti-c-Met antibody, wherein the threshold comprises only one of (i)-(xiv).

In another embodiment of any of the preceding aspects, the bispecific anti-c-Met/anti-EpCAM antibody is Ab#5, Ab#7, or Ab#13 as disclosed in PCT publication No. WO/2014/138449. In certain embodiments, the bispecific anti-c-Met/anti-EpCAM antibody comprises a light chain amino acid sequence set forth in SEQ ID NO: 400. In certain embodiments, the bispecific anti-c-Met/anti-EpCAM antibody further comprises a heavy chain amino acid sequence select from the group consisting of SEQ ID NOs: 407, 410, and 417.

In an embodiment, the bispecific anti-c-Met/anti-EpCAM antibody comprises a light chain amino acid sequence set forth in SEQ ID NO: 400 and a heavy chain amino acid sequence set forth in SEQ ID NO: 407.

In an embodiment, the bispecific anti-c-Met/anti-EpCAM antibody comprises a light chain amino acid sequence set forth in SEQ ID NO: 400 and a heavy chain amino acid sequence set forth in SEQ ID NO: 410.

In an embodiment, the bispecific anti-c-Met/anti-EpCAM antibody comprises a light chain amino acid sequence set forth in SEQ ID NO: 400 and a heavy chain amino acid sequence set forth in SEQ ID NO: 417.

In another embodiment, the HGF ISH score is obtained using one or more nucleic acid ISH probes that hybridize specifically to a nucleic acid that comprises the sequence of nucleotides 346-1806 of the nucleotide sequence set forth in GenBank accession number NM_000601.4 (the full GenBank sequence is set forth below, with nucleotides 346-1806 underlined. In another embodiment, the c-Met IHC scores are obtained using at least one anti-c-Met antibody selected from clone SP44 (Ventana Medical Systems), Clone Met4 (DAKO, Carpinteria, Calif.), and Clone Met (D1C2) XP (Cell Signaling Technologies, Danvers, Mass.). In another embodiment, the EpCAM IHC scores are obtained using at least one anti-EpCAM antibody selected from clone VU1D9 (Cell Signaling Technologies; Cat# #2929) and Clone MOC-31 (DAKO M3525).

GenBank accession number NM_000601.4 nucleotide sequence:

| | |
|---|---|
| gggagttcag acctagatct ttccagttaa tcacacaaca aacttagctc atcgcaataa | 60 |
| aaagcagctc agagccgact ggctctttta ggcactgact ccgaacagga ttctttcacc | 120 |
| caggcatctc ctccagaggg atccgccagc ccgtccagca gcaccatgtg ggtgaccaaa | 180 |
| ctcctgccag ccctgctgct gcagcatgtc ctcctgcatc tcctcctgct ccccatcgcc | 240 |
| atcccctatg cagagggaca aaggaaaaga agaaatacaa ttcatgaatt caaaaaatca | 300 |
| gcaaagacta ccctaatcaa aatagatcca gcactgaaga taaaaaccaa aaaagtgaat | 360 |
| actgcagacc aatgtgctaa tagatgtact aggaataaag gacttccatt cacttgcaag | 420 |
| gcttttgttt ttgataaagc aagaaaacaa tgcctctggt tccccttcaa tagcatgtca | 480 |
| agtggagtga aaaaagaatt tggccatgaa tttgacctct atgaaaacaa agactacatt | 540 |
| agaaactgca tcattggtaa aggacgcagc tacaagggaa cagtatctat cactaagagt | 600 |
| ggcatcaaat gtcagccctg gagttccatg ataccacacg aacacagctt tttgccttcg | 660 |
| agctatcggg gtaaagacct acaggaaaac tactgtcgaa atcctcgagg ggaagaaggg | 720 |
| ggaccctggt gtttcacaag caatccagag gtacgctacg aagtctgtga cattcctcag | 780 |
| tgttcagaag ttgaatgcat gacctgcaat ggggagagtt atcgaggtct catggatcat | 840 |
| acagaatcag gcaagatttg tcagcgctgg gatcatcaga caccacaccg gcacaaattc | 900 |
| ttgcctgaaa gatatcccga caagggcttt gatgataatt attgccgcaa tcccgatggc | 960 |
| cagccgaggc catggtgcta tactcttgac cctcacaccc gctgggagta ctgtgcaatt | 1020 |
| aaaacatgcg ctgacaatac tatgaatgac actgatgttc ctttggaaac aactgaatgc | 1080 |
| atccaaggtc aaggagaagg ctacaggggc actgtcaata ccatttggaa tggaattcca | 1140 |
| tgtcagcgtt gggattctca gtatcctcac gagcatgaca tgactcctga aaatttcaag | 1200 |
| tgcaaggacc tacgagaaaa ttactgccga aatccagatg ggtctgaatc accctggtgt | 1260 |
| tttaccactg atccaaacat ccgagttggc tactgctccc aaattccaaa ctgtgatatg | 1320 |
| tcacatggac aagattgtta tcgtgggaat ggcaaaaatt atatgggcaa cttatcccaa | 1380 |
| acaagatctg gactaacatg ttcaatgtgg gacaagaaca tggaagactt acatcgtcat | 1440 |
| atcttctggg aaccagatgc aagtaagctg aatgagaatt actgccgaaa tccagatgat | 1500 |
| gatgctcatg gaccctggtg ctacacggga aatccactca ttccttggga ttattgccct | 1560 |
| atttctcgtt gtgaaggtga taccacacct acaatagtca atttagacca tcccgtaata | 1620 |
| tcttgtgcca aaacgaaaca attgcgagtt gtaaatggga ttccaacacg aacaaacata | 1680 |
| ggatggatgg ttagtttgag atacagaaat aaacatatct gcggaggatc attgataaag | 1740 |
| gagagttggg ttcttactgc acgacagtgt ttcccttctc gagacttgaa agattatgaa | 1800 |
| gcttggcttg gaattcatga tgtccacgga agaggagatg agaaatgcaa acaggttctc | 1860 |
| aatgtttccc agctggtata tggccctgaa ggatcagatc tggttttaat gaagcttgcc | 1920 |
| aggcctgctg tcctggatga ttttgttagt acgattgatt tacctaatta tggatgcaca | 1980 |
| attcctgaaa agaccagttg cagtgtttat ggctggggct acactggatt gatcaactat | 2040 |
| gatggcctat tacgagtggc acatctctat ataatgggaa atgagaaatg cagccagcat | 2100 |
| catcgaggga aggtgactct gaatgagtct gaaatatgtg ctggggctga aaagattgga | 2160 |
| tcaggaccat gtgaggggga ttatggtggc ccacttgttt gtgagcaaca taaaatgaga | 2220 |
| atggttcttg gtgtcattgt tcctggtcgt ggatgtgcca ttccaaatcg tcctggtatt | 2280 |
| tttgtccgag tagcatatta tgcaaaatgg atacacaaaa ttattttaac atataaggta | 2340 |
| ccacagtcat agctgaagta agtgtgtctg aagcacccac caatacaact gtcttttaca | 2400 |
| tgaagatttc agagaatgtg gaatttaaaa tgtcacttac aacaatccta agacaactac | 2460 |
| tggagagtca tgtttgttga aattctcatt aatgtttatg ggtgttttct gttgttttgt | 2520 |
| ttgtcagtgt tattttgtca atgttgaagt gaattaaggt acatgcaagt gtaataacat | 2580 |
| atctcctgaa gatacttgaa tggattaaaa aaacacacag gtatatttgc tggatgataa | 2640 |
| agatttcatg ggaaaaaaaa tcaattaatc tgtctaagct gctttctgat gttggtttct | 2700 |
| taataatgag taaaccacaa attaaatgtt attttaacct caccaaaaca atttatacct | 2760 |
| tgtgtcccta aattgtagcc ctatattaaa ttatattaca tttcaaaaaa aaaaaaaaaa | 2820 |

In another embodiment of the preceding aspects, the cancer is bladder, breast, cervical, colorectal, gastric, gastroesophageal, esophageal, head and neck, liver, lung (e.g., non-small cell lung cancer (NSCLC)), ovarian, pancreatic, prostrate, renal or thyroid cancer.

In another embodiment of the preceding aspects, if the threshold is not met, the patient receives treatment with an anti-cancer therapeutic that does not comprise a bispecific anti-EpCAM/anti-c-Met antibody.

In another embodiment, the treatment, when administered to a plurality of the selected patients, produces an increase in the frequency in the treated patients of at least one therapeutic effect selected from the group consisting of reduction in size of a tumor, reduction in number of metastatic lesions over time, complete response, partial response, stable disease, increase in overall response rate, or a pathologic complete response, compared to a comparator population of patients who receive the treatment without the selection. In another embodiment the treatment, when administered to a plurality of the selected patients, results in an increase in rates of progression-free survival or overall survival for the treated patients compared to a comparator population of patients who receive the treatment without the selection. In another embodiment, the treatment, when administered to a plurality of the selected patients, results in an improved quality of life for the treated patients compared to a comparator population of patients who receive the treatment without the selection. In the three immediately preceding embodiments, the comparator population may be a disease-matched comparator population (e.g., a control population).

### BRIEF DESCRIPTION OF THE DRAWINGS

FIG. 1 shows exemplary hepatocyte growth factor (HGF) RNA staining pattern in cell lines representative of each ordinal score for RNA ISH. The corresponding expression of mRNA by Robust Multi-array Average (RMA), normalized using quantile normalization, is presented at the bottom of the image. Shown are A549 (epithelial lung carcinoma, score 0, FIG. 1A), H2023 (non-small cell lung cancer, adenocarcinoma score 1, FIG. 1B), U87 (brain, glioblastoma, score 2, FIG. 1C), RKO (colon, score 3, FIG. 1D), and A204 (muscle—rhabdomyosarcoma, score 4, FIG. 1E) cancer cell lines. The inset shows a higher magnification of a particular region of the same cell line.

FIG. 2 shows exemplary hepatocyte growth factor (HGF) RNA staining pattern in cancer tissues representative of each of the ordinal scores for RNA ISH. Shown are colon (score 0, FIG. 2A), colon (score 1, FIG. 2B), gastric (score 2, FIG. 2C), lung (score 3, FIG. 2D), and lung (score 4 FIG. 2E) human cancer tissue sections. The inset shows a higher magnification of a particular region of the same tissue sample.

FIG. 3 shows exemplary c-Met IHC staining in cell lines representative of each of the ordinal scores used to score c-Met IHC along with estimated receptor density by quantitative flow cytometry. Shown are TOV-112D (ovarian cancer, FIG. 3A), NCI-H1703 (non-small-cell lung cancer, FIG. 3B), A549 (epithelial lung, FIG. 3C), and NCI-H441 (lung papillary adenocarcinoma, FIG. 3D) cell lines. The inset shows a higher magnification of a particular region of the same cell line.

FIG. 4 shows exemplary c-Met IHC staining in cancer tissues representative of each of the ordinal scores for c-Met IHC. Shown are lung (score 0, FIG. 4A), gastric (score 1, FIG. 4B), colon (score 2, FIG. 4C), and gastric (score 3, FIG. 4D) human cancer tissue. The inset shows a higher magnification of a particular region of the same tissue sample.

FIG. 5 shows an exemplary HGF IHC staining in cell lines representative of each of the ordinal scores used to score HGF IHC. Shown are A549 (epithelial lung, score 0, FIG. 5A), H2023 (non-small cell lung cancer, adenocarcinoma, score 1, FIG. 5B), U87 (brain, glioblastoma, score 2, FIG. 5C), KP4 (pancreatic cancer, score 3, FIG. 5D), and HCC827-HGF (lung adenocarcinoma, score 4, FIG. 5E) cell lines. The inset shows a higher magnification of a particular region of the same cell line.

FIG. 6 shows an exemplary HGF IHC staining in NSCLC cancer tissue samples representative of three of the ordinal scores for HGF IHC. Shown are a tissue with score 0 (HGF negative, FIG. 6A), a tissue section with score 1 (FIG. 6B), and a tissue section with score 3 (FIG. 6C).

FIG. 7 shows an exemplary EpCAM IHC staining in cell lines representative of each of the ordinal scores used to score EpCAM IHC along with estimated receptor density by quantitative flow cytometry. Shown are A549 (epithelial lung, score 0, FIG. 7A), ACHN (renal cell carcinoma, score 1, FIG. 7B), SK-OV-3 (ovarian adenocarcinoma, score 2, FIG. 7C), and H1993 (non-small cell lung cancer, score 3, FIG. 7D) cell lines. The inset shows a higher magnification of a particular region of the same cell line.

FIG. 8 shows an exemplary EpCAM IHC staining in cancer tissues representative of each of the ordinal scores for EpCAM IHC. Shown are gastric (score 0, FIG. 8A), gastric (score 1, FIG. 8B), lung (score 2, FIG. 8C) and colon (score 3, FIG. 8D) human cancer tissue. The inset shows a higher magnification of a particular region of the same tissue sample.

FIG. 9 shows exemplary in vivo activity of Ab#7 (triangles), OA-5D5 (squares) and negative control (“vehicle”, circles) in preclinical xenograft models of cancer that express distinct levels of c-Met, HGF, and EpCAM. In FIG. 9A is shown the results of a xenograft model using U87-MG brain cells having a c-Met 1+, EpCAM 0 (negative), HGF 2+ profile, wherein Ab#7 inhibits tumor cell growth. In FIG. 9B is shown the results of a xenograft model of H441 lung papillary adenocarcinoma cells having a c-Met 3+, EpCAM 3+, and HGF 0 (negative) profile, wherein both OA-5D5 and Ab#7 inhibited tumor cell growth. In FIG. 9C is shown the results of a xenograft model of HCC-827-HGF lung adenocarcinoma cells having a c-Met2+, EpCAM3+, HGF4+ profile, wherein only Ab#7 strongly inhibited tumor cell growth. Data are shown as a function of tumor volume vs. days post-implantation.

### DETAILED DESCRIPTION

Provided herein are methods suitable for a) predicting patient responsiveness to, or b) selecting patients for treatment with, a c-Met targeted or other HGF-inhibitory treatment e.g., a bispecific a c-Met targeted and EpCAM targeted bispecific antibody (e.g. a bispecific antibody such as Ab#5, Ab#7, or Ab#10 as disclosed in WO/2014138449), using particular biomarker scores obtained from a cancer cell sample from of the patient (i.e., c-Met, HGF, EpCAM or any combination thereof).

### Definitions

“c-Met”, also called “MET” and hepatocyte growth factor receptor (HGFR), is a protein that in humans is encoded by the MET gene, as described, e.g., in U.S. Pat. No. 7,605,127.

“c-Met inhibitor” indicates a therapeutic agent that inhibits, downmodulates, suppresses or downregulates activity or expression of c-Met, e.g. an agent that does one or more of the following: reduces cellular c-Met levels; reduces ligand binding to c-Met, and reduces c-Met-mediated intracellular signal transduction. The term is intended to include small molecule kinase inhibitors, antibodies, interfering RNAs (shRNA, siRNA), soluble receptors, and the like. An exemplary c-Met inhibitor is an antibody, e.g., an anti-c-Met antibody.

An “anti-c-Met antibody” is an antibody that immunospecifically binds to the ectodomain of c-Met. The antibody may be an isolated antibody. Exemplary anti-c-Met antibodies inhibit phosphorylation of c-Met mediated by ligand (e.g., HGF), and some may also inhibit transactivation of c-Met activity mediated by activation of another receptor tyrosine kinase. Anti-c-Met antibodies may also inhibit auto-phosphorylation of c-Met due to aberrantly high expression of c-Met, e.g., in c-Met gene-amplified settings where c-MET may be thus activated, e.g., via homodimerization. An exemplary anti-c-Met antibody is 224G11-TH7-Hz3, disclosed in U.S. Patent Pub. No. 2011/0097262: SEQ ID NO:4 (V_{H} domain), 10 (V_{L} domain), and 28 (hinge region); these sequences are shown below. Anti-c-Met antibodies also include bispecific antibodies, wherein the antibody binds c-Met and another antigen. Examples of such antibodies include Ab#5, Ab#7, or Ab#10 as disclosed in WO/2014138449, which bind both c-Met and EpCAM.

| SEQ ID NO: 4 of U.S. Patent Pub. No. 2011/0097262: | |
|---|---|
| QVQLVQSGAE VKKPGASVKV SCKASGYIFT AYTMHWVRQA PGQGLEWMGW IKPNNGLANY | 60 |
| AQKFQGRVTM TRDTSISTAY MELSRLRSDD TAVYYCARSE ITTEFDYWGQ GTLVTVSS | 118 |
| SEQ ID NO: 10 of U.S. Patent Pub. No. 2011/0097262: | |
| DIVMTQSPDS LAVSLGERAT INCKSSESVD SYANSFLHWY QQKPGQPPKL LIYRASTRES | 60 |
| GVPDRFSGSG SGTDFTLTIS SLQAEDVAVY YCQQSKEDPL TFGGGTKVEI KR | 112 |
| SEQ ID NO: 28 of U.S. Patent Pub. No. 2011/0097262: | |
| PKSCDCHCPP CP | 12 |

An “antibody,” is a protein consisting of one or more polypeptides comprising binding domains substantially encoded by immunoglobulin genes or fragments of immunoglobulin genes, wherein the protein immunospecifically binds to an antigen. One type of naturally occurring immunoglobulin structural unit (e.g., an IgG) comprises a tetramer that is composed of two identical pairs of polypeptide chains, each pair having one “light” (about 25 kD) and one “heavy” chain (about 50-70 kD). “VL” and VH″ refer to the variable regions of these light and heavy chains respectively. “Antibodies” include intact proteins as well as antigen-binding fragments, which may be produced by digestion of intact proteins, e.g., with various peptidases, or may be synthesized de novo either chemically or using recombinant DNA expression technology. Such fragments include, for example, F(ab)₂ dimers and Fab monomers, and single chain antibodies. Single chain antibodies exist, generally due to genetic engineering, as a single polypeptide chain, e.g., single chain Fv antibodies (scFv) in which a V_{H} fragment and a V_{L} fragment are joined together (directly or through a peptide linker) to form a continuous polypeptide that retains immunospecific binding activity.

“CH1 domain” refers to the heavy chain immunoglobulin constant domain located between the VH domain and the hinge. It spans EU positions 118-215. A CH1 domain may be a naturally occurring CH1 domain, or a naturally occurring CH1 domain in which one or more amino acids (“aas”) have been substituted, added or deleted, provided that the CH1 domain has the desired biological properties. A desired biological activity may be a natural biological activity, an enhanced biological activity or a reduced biological activity relative to the naturally occurring sequence.

“CH2 domain” refers to the heavy chain immunoglobulin constant domain that is located between the hinge and the CH3 domain. As defined here, it spans EU positions 237-340. A CH2 domain may be a naturally occurring CH2 domain, or a naturally occurring CH2 domain in which one or more aas have been substituted, added or deleted, provided that the CH2 domain has the desired biological properties. A desired biological activity may be a natural biological activity, an enhanced biological activity or a reduced biological activity relative to that of the naturally occurring domain.

“CH3 domain” refers to the heavy chain immunoglobulin constant domain that is located C-terminally of the CH2 domain and spans approximately 110 residues from the N-terminus of the CH2 domain, e.g., about positions 341-446b (EU numbering system). A CH3 domain may be a naturally occurring CH3 domain, or a naturally occurring CH3 domain in which one or more aas have been substituted, added or deleted, provided that the CH3 domain has the desired biological properties. A desired biological activity may be a natural biological activity, an enhanced biological activity or a reduced biological activity relative to that of the naturally occurring domain. A CH3 domain may or may not comprise a C-terminal lysine.

“Fab” refers to the antigen binding portion of an antibody, comprising two chains: a first chain that comprises a VH domain and a CH1 domain and a second chain that comprises a VL domain and a CL domain. Although a Fab is typically described as the N-terminal fragment of an antibody that was treated with papain and comprises a portion of the hinge region, it is also used herein as referring to a binding domain wherein the heavy chain does not comprise a portion of the hinge.

“Fc region” refers to the portion of a single immunoglobulin heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc region comprises at least a hinge, a CH2 domain, and a CH3 domain. Two Fc regions that are dimerized are referred to as “Fc” or “Fc dimer.” An Fc region may be a naturally occurring Fc region, or a naturally occurring Fc region in which one or more aas have been substituted, added or deleted, provided that the Fc region has the desired biological properties. A desired biological activity may be a natural biological activity, an enhanced biological activity or a reduced biological activity relative to that of the naturally occurring domain.

“Gly-Ser linker” or “Gly-Ser peptide” refers to a peptide that consists of glycine and serine residues. An exemplary Gly-Ser peptide comprises the amino acid sequence (Gly4 Ser)n, wherein n=1, 2, 3, 4, 5, 6, 7, 8, 9, 10, 11, 12, 13, 14, 15, 16, 17, 18, 19, 20 or more. In certain embodiments, n is a number between 1 and 5, n is a number between 6 and 10, n is a number between 11 and 15, n is a number between 16 and 20, n is a number between 21 and 25, or n is a number between 26 and 30.

“Hinge” or “hinge region” or “hinge domain” refers to the flexible portion of a heavy chain located between the CH1 domain and the CH2 domain. It is approximately 25 aas long, and is divided into an “upper hinge,” a “middle hinge” or “core hinge,” and a “lower hinge.” A hinge may be a naturally occurring hinge, or a naturally occurring hinge in which one or more aas have been substituted, added or deleted, provided that the hinge has the desired biological properties. A desired biological activity may be a natural biological activity, an enhanced biological activity or a reduced biological activity relative to the naturally occurring sequence. A “hinge subdomain” refers to the upper hinge, middle (or core) hinge or the lower hinge. The complete hinge consists of the upper hinge subdomain, middle hinge subdomain and lower hinge subdomain in amino to carboxy terminal order and without intervening sequences.

“Linker” refers to one or more aas connecting two domains or regions together. A linker may be flexible to allow the domains being connected by the linker to form a proper three dimensional structure thereby allowing them to have the required biological activity. A linker connecting the VH and the VL of an scFv is referred to herein as an “scFv linker.” A linker connecting the N-terminus of a VH domain or the C-terminus of the CH3 domain to a second VH or VL domain, e.g., that of an scFv, is referred to as a “connecting linker.”

A “TFc” or “tandem Fc” refers to an entity comprising in an amino to carboxyl terminal order: a first Fc region, which is linked at its C-terminus to the N-terminus of a TFc linker, which is linked at its C-terminus to the N-terminus of a second Fc region, wherein the first and the second Fc regions associate to form an Fc.

“TFcA” refers to a tandem Fc antibody. A TFcA may be a monovalent or monospecific TFcA, e.g., comprising a single binding site. A TFcA may also be a bispecific TFcA, which is referred to herein as a TFcBA. A TFcA may be monoclonal.

“TFcBA” refers to a tandem Fc bispecific antibody, an artificial hybrid protein comprising at least two different binding moieties or domains and thus at least two different binding sites (e.g., two different antibody binding sites), wherein one or more of the pluralities of the binding sites are covalently linked, e.g., via peptide bonds, to each other.

A TFcBA may comprise a heavy chain comprising in amino to carboxyl-terminal order:
(i) a first VH domain, a TFc, a connecting linker, and a second VH domain;
(ii) a first VH domain, a CH1 domain, a TFc, a connecting linker, and a second VH domain;
(iii) a first VH domain, a CH1 domain, a TFc, a connecting linker, a second VH domain, an scFv linker and a second VL domain, wherein the second VH and VL domains associate to form a second binding site;
(iv) a first VH domain, a TFc, a connecting linker, a second VH domain, and a CH1 domain;
(v) a first VH domain, a first CH1 domain, a TFc, a connecting linker, a second VH domain and a second CH1 domain;
(vi) a first VH domain, a first scFv linker, a first VL domain, a TFc, a connecting linker, and a second VH domain, wherein the first VL and VH domains associate to form a first binding site;
(vii) a first VH domain, a first scFv linker, a first VL domain, a TFc, a connecting linker, a second VH domain, and a CH1 domain, wherein the first VL and VH domains associate to form a first binding site; and
(viii) a first VH domain, a first scFv linker, a first VL domain, a TFc, a connecting linker, a second VH domain, a second scFv linker, and a second VL domain, wherein the first VH and VL domains form a first binding site and the second VH and VL domains form a second binding site.

A TFcBA of (i-)(v) may further comprise a light chain comprising a first VL domain and optionally a CL domain located at the C-terminus of the VL domain, wherein the first VH and VL domains associate to form a first binding site. A TFcBA of (i), (ii), (iv)-(vii) may comprise a light chain comprising a second VL domain and optionally a CL domain located at the C-terminus of the VL domain, wherein the first VH and VL domains associate to form a second binding site

An exemplary TFcBA described herein is an anti-c-Met+anti-EpCAM TFcBA, which is a polyvalent bispecific antibody that comprises a first binding site binding specifically to a c-Met protein, e.g., a human c-Met protein, and one or more second binding sites binding specifically to an EpCAM protein, e.g., a human EpCAM protein. When a TFcBA name comprises two antigens separated by a plus sign (+) this indicates that the binding sites for the two antigens may be in either relative amino to carboxy orientation in the molecule, whereas when the TFcBA name comprises two antigen binding site names separated by a slash (/) the antigen binding site to the left of the slash is amino terminal to the antigen binding site to the right of the slash. A TFcBA may be a bivalent binding protein, a trivalent binding protein, a tetravalent binding protein or a binding protein with more than 4 binding sites. An exemplary TFcBA is a bivalent bispecific antibody, i.e., an antibody that has 2 binding sites, each binding to a different antigen or epitope. In certain embodiments, the N-terminal binding site of a TFcBA is a Fab and the C-terminal binding site is an scFv.

“Module” refers to a structurally and/or functionally distinct part of a TFcA, such a binding site (e.g., an scFv domain or a Fab domain) and the TFc. Modules provided herein can be rearranged (by recombining sequences encoding them, either by recombining nucleic acids or by complete or fractional de novo synthesis of new polynucleotides) in numerous combinations with other modules to produce a wide variety of TFcAs, e.g., as disclosed herein.

“Immunospecific” or “immunospecifically” refer to binding via domains substantially encoded by the variable region(s) of immunoglobulin genes or fragments of immunoglobulin genes to one or more epitopes of a protein or other molecule of interest, but which do not specifically bind to unrelated molecules in a sample containing a mixed population of antigenic molecules. Typically, an antibody binds immunospecifically to a cognate antigen with a K_{D} with a value of no greater than 100 nM, or preferably no greater than 50 nM, (a higher K_{D} value indicates weaker binding) as measured e.g., by a surface plasmon resonance assay or a cell binding assay.

“Specific hybridization” refers to a nucleic acid molecule, such as a probe, that forms an anti-parallel double-stranded structure with a target region under certain hybridizing conditions, while failing to form such a structure when incubated with a different target polynucleotide or another region in the polynucleotide or with a polynucleotide lacking the desired target under the same hybridizing conditions. Typically, the nucleic acid molecule specifically hybridizes to the target region under conventional high stringency conditions. Appropriate stringency conditions that promote DNA hybridization are, for example, 6.0× sodium chloride/sodium citrate (SSC) at about 45 degrees C., followed by a wash of 2.0× SSC at 50 degrees C., and are known to those skilled in the art or can be found in Current Protocols in Molecular Biology, John Wiley & Sons, N.Y., 1989, 6.3.1-6.3.6. For example, the salt concentration in the wash step can be selected from a low stringency of about 2.0×SSC at 50 degrees C. to a high stringency of about 0.2×SSC at 50 degrees C.

The terms “suppress”, “suppression”, “inhibit” and “inhibition” as used herein, refer to any statistically significant decrease in biological activity (e.g., tumor cell growth), including full blocking of the activity. For example, “inhibition” can refer to a decrease of about 10%, 20%, 30%, 40%, 50%, 60%, 70%, 80%, 90%, or 100% in biological activity.

“FFPE” indicates formalin fixation and paraffin embedding (or, formalin fixed and paraffin embedded).

“Fl-IHC” indicates fluorescence-based quantitative immunohistochemistry.

“HGF” indicates any and all isotypes of hepatocyte growth factor, the naturally occurring ligand to c-Met. Hepatocyte growth factor (HGF) is a c-Met ligand that activates c-Met in living cells (i.e., triggers phosphorylation of tyrosine residues in the cytoplasmic domain of c-Met), thereby initiating intracellular signaling. Such signaling can promote cellular activities, e.g., the growth, proliferation, migration and metastasis of cancer cells. This activation may occur in an autocrine fashion, in which the HGF produced by a cell activates the same cell, or it may occur in a paracrine fashion, in which HGF produced by one cell (e.g., a stromal cell in a tumor) activates neighboring cells (e.g., tumor cells). Accordingly, it is desirable to measure HGF expression in both tumor cells and stromal cells in the same biopsy. This can be achieved by visualizing HGF transcripts (e.g., in FFPE patient samples) using RNA in situ hybridization (RNA-ISH) and scoring patient samples based on the observed hybridization levels. One non-limiting example of human HGF nucleotide and amino acid sequence are shown below (GenBank M60718.1).

### HGF Nucleotide Sequence Example:

| | |
|---|---|
| actgactccg aacaggattc tttcacccag gcatctcctc cagagggatc cgccagcccg | 60 |
| tccagcagca ccatgtgggt gaccaaactc ctgccagccc tgctgctgca gcatgtcctc | 120 |
| ctgcatctcc tcctgctccc catcgccatc ccctatgcag agggacaaag gaaaagaaga | 180 |
| aatacaattc atgaattcaa aaaatcagca aagactaccc taatcaaaat agatccagca | 240 |
| ctgaagataa aaaccaaaaa agtgaatact gcagaccaat gtgctaatag atgtactagg | 300 |
| aataaaggac ttccattcac ttgcaaggct tttgtttttg ataaagcaag aaaacaatgc | 360 |
| ctctggttcc ccttcaatag catgtcaagt ggagtgaaaa aagaatttgg ccatgaattt | 420 |
| gacctctatg aaaacaaaga ctacattaga aactgcatca ttggtaaagg acgcagctac | 480 |
| aagggaacag tatctatcac taagagtggc atcaaatgtc agccctggag ttccatgata | 540 |
| ccacacgaac acagcttttt gccttcgagc tatcggggta aagacctaca ggaaaactac | 600 |
| tgtcgaaatc ctcgagggga agaaggggga ccctggtgtt tcacaagcaa tccagaggta | 660 |
| cgctacgaag tctgtgacat tcctcagtgt tcagaagttg aatgcatgac ctgcaatggg | 720 |
| gagagttatc gaggtctcat ggatcataca gaatcaggca agatttgtca gcgctgggat | 780 |
| catcagacac cacaccggca caaattcttg cctgaaagat atcccgacaa gggctttgat | 840 |
| gataattatt gccgcaatcc cgatggccag ccgaggccat ggtgctatac tcttgaccct | 900 |
| cacacccgct gggagtactg tgcaattaaa acatgcgctg acaatactat gaatgacact | 960 |
| gatgttcctt tggaaacaac tgaatgcatc caaggtcaag gagaaggcta caggggcact | 1020 |
| gtcaatacca tttggaatgg aattccatgt cagcgttggg attctcagta tcctcacgag | 1080 |
| catgacatga ctcctgaaaa tttcaagtgc aaggacctac gagaaaatta ctgccgaaat | 1140 |
| ccagatgggt ctgaatcacc ctggtgtttt accactgatc caaacatccg agttggctac | 1200 |
| tgctcccaaa ttccaaactg tgatatgtca catggacaag attgttatcg tgggaatggc | 1260 |
| aaaaattata tgggcaactt atcccaaaca agatctggac taacatgttc aatgtgggac | 1320 |
| aagaacatgg aagacttaca tcgtcatatc ttctgggaac cagatgcaag taagctgaat | 1380 |
| gagaattact gccgaaatcc agatgatgat gctcatggac cctggtgcta cacgggaaat | 1440 |
| ccactcattc cttgggatta ttgccctatt tctcgttgtg aaggtgatac cacacctaca | 1500 |
| atagtcaatt tagaccatcc cgtaatatct tgtgccaaaa cgaaacaatt gcgagttgta | 1560 |
| aatgggattc caacacgaac aaacatagga tggatggtta gtttgagata cagaaataaa | 1620 |
| catatctgcg gaggatcatt gataaaggag agttgggttc ttactgcacg acagtgtttc | 1680 |
| ccttctcgag acttgaaaga ttatgaagct tggcttggaa ttcatgatgt ccacggaaga | 1740 |
| ggagatgaga aatgcaaaca ggttctcaat gtttcccagc tggtatatgg ccctgaagga | 1800 |
| tcagatctgg ttttaatgaa gcttgccagg cctgctgtcc tggatgattt tgttagtacg | 1860 |
| attgatttac ctaattatgg atgcacaatt cctgaaaaga ccagttgcag tgtttatggc | 1920 |
| tggggctaca ctggattgat caactatgat ggcctattac gagtggcaca tctctatata | 1980 |
| atgggaaatg agaaatgcag ccagcatcat cgagggaagg tgactctgaa tgagtctgaa | 2040 |
| atatgtgctg gggctgaaaa gattggatca ggaccatgtg agggggatta tggtggccca | 2100 |
| cttgtttgtg agcaacataa aatgagaatg gttcttggtg tcattgttcc tggtcgtgga | 2160 |
| tgtgccattc caaatcgtcc tggtattttt gtccgagtag catattatgc aaaatggata | 2220 |
| cacaaaatta ttttaacata taaggtacca cagtcatagc tgaagtaagt gtgtctgaag | 2280 |
| cacccaccaa tacaactgtc ttttac | 2306 |

### HGF Amino Acid Sequence Example:

| | |
|---|---|
| MWVTKLLPAL LLQHVLLHLL LLPIAIPYAE GQRKRRNTIH EFKKSAKTTL IKIDPALKIK | 60 |
| TKKVNTADQC ANRCTRNKGL PFTCKAFVFD KARKQCLWFP FNSMSSGVKK EFGHEFDLYE | 120 |
| NKDYIRNCII GKGRSYKGTV SITKSGIKCQ PWSSMIPHEH SFLPSSYRGK DLQENYCRNP | 180 |
| RGEEGGPWCF TSNPEVRYEV CDIPQCSEVE CMTCNGESYR GLMDHTESGK ICQRWDHQTP | 240 |
| HRHKFLPERY PDKGFDDNYC RNPDGQPRPW CYTLDPHTRW EYCAIKTCAD NTMNDTDVPL | 300 |
| ETTECIQGQG EGYRGTVNTI WNGIPCQRWD SQYPHEHDMT PENFKCKDLR ENYCRNPDGS | 360 |
| ESPWCFTTDP NIRVGYCSQI PNCDMSHGQD CYRGNGKNYM GNLSQTRSGL TCSMWDKNME | 420 |
| DLHRHIFWEP DASKLNENYC RNPDDDAHGP WCYTGNPLIP WDYCPISRCE GDTTPTIVNL | 480 |
| DHPVISCAKT KQLRVVNGIP TRTNIGWMVS LRYRNKHICG GSLIKESWVL TARQCFPSRD | 540 |
| LKDYEAWLGI HDVHGRGDEK CKQVLNVSQL VYGPEGSDLV LMKLARPAVL DDFVSTIDLP | 600 |
| NYGCTIPEKT SCSVYGWGYT GLINYDGLLR VAHLYIMGNE KCSQHHRGKV TLNESEICAG | 660 |
| AEKIGSGPCE GDYGGPLVCE QHKMRMVLGV IVPGRGCAIP NRPGIFVRVA YYAKWIHKII | 720 |
| LTYKVPQS | 728 |

“PCR” indicates polymerase chain reaction in any experimental embodiment of the method first set forth in Mullis, 1987, U.S. Pat. No. 4,683,202).

“qIHC” indicates chromogenic quantitative immunohistochemistry.

“RT-PCR” indicates reverse transcription followed by PCR of the resulting reverse transcripts.

A “sample”, “tumor cell sample,” “cancer cell sample” “patient sample”, or “sample from a patient”, as used herein, is meant a sample comprising tumor cells from the patient. Such a sample may be, e.g., from a biopsy of a tumor, a tissue sample, or circulating tumor cells from the blood.

“EpCAM” refers to epithelial cell adhesion molecule. Exemplary human EpCAM nucleic acid and protein sequences are set forth in RefSeqGene Gene ID: 4072 and GenBank Accession Number: NP 002345.2, respectively, which sequences are shown below:

### EpCAM Nucleotide Sequence Example:

| | |
|---|---|
| AACTGCAGCG CCGGGGCTGG GGGAGGGGAG CCTACTCACT CCCCCAACTC CCGGGCGGTG | 60 |
| ACTCATCAAC GAGCACCAGC GGCCAGAGGT GAGCAGTCCC GGGAAGGGGC CGAGAGGCGG | 120 |
| GGCCGCCAGG TCGGGCAGGT GTGCGCTCCG CCCCGCCGCG CGCACAGAGC GCTAGTCCTT | 180 |
| CGGCGAGCGA GCACCTTCGA CGCGGTCCGG GGACCCCCTC GTCGCTGTCC TCCCGACGCG | 240 |
| GACCCGCGTG CCCCAGGCCT CGCGCTGCCC GGCCGGCTCC TCGTGTCCCA CTCCCGGCGC | 300 |
| ACGCCCTCCC GCGAGTCCCG GGCCCCTCCC GCGCCCCTCT TCTCGGCGCG CGCGCAGCAT | 360 |
| GGCGCCCCCG CAGGTCCTCG CGTTCGGGCT TCTGCTTGCC GCGGCGACGG CGACTTTTGC | 420 |
| CGCAGCTCAG GAAGAATGTG TCTGTGAAAA CTACAAGCTG GCCGTAAACT GCTTTGTGAA | 480 |
| TAATAATCGT CAATGCCAGT GTACTTCAGT TGGTGCACAA AATACTGTCA TTTGCTCAAA | 540 |
| GCTGGCTGCC AAATGTTTGG TGATGAAGGC AGAAATGAAT GGCTCAAAAC TTGGGAGAAG | 600 |
| AGCAAAACCT GAAGGGGCCC TCCAGAACAA TGATGGGCTT TATGATCCTG ACTGCGATGA | 660 |
| GAGCGGGCTC TTTAAGGCCA AGCAGTGCAA CGGCACCTCC ATGTGCTGGT GTGTGAACAC | 720 |
| TGCTGGGGTC AGAAGAACAG ACAAGGACAC TGAAATAACC TGCTCTGAGC GAGTGAGAAC | 780 |
| CTACTGGATC ATCATTGAAC TAAAACACAA AGCAAGAGAA AAACCTTATG ATAGTAAAAG | 840 |
| TTTGCGGACT GCACTTCAGA AGGAGATCAC AACGCGTTAT CAACTGGATC CAAAATTTAT | 900 |
| CACGAGTATT TTGTATGAGA ATAATGTTAT CACTATTGAT CTGGTTCAAA ATTCTTCTCA | 960 |
| AAAAACTCAG AATGATGTGG ACATAGCTGA TGTGGCTTAT TATTTTGAAA AAGATGTTAA | 1020 |
| AGGTGAATCC TTGTTTCATT CTAAGAAAAT GGACCTGACA GTAAATGGGG AACAACTGGA | 1080 |
| TCTGGATCCT GGTCAAACTT TAATTTATTA TGTTGATGAA AAAGCACCTG AATTCTCAAT | 1140 |
| GCAGGGTCTA AAAGCTGGTG TTATTGCTGT TATTGTGGTT GTGGTGATAG CAGTTGTTGC | 1200 |
| TGGAATTGTT GTGCTGGTTA TTTCCAGAAA GAAGAGAATG GCAAAGTATG AGAAGGCTGA | 1260 |
| GATAAAGGAG ATGGGTGAGA TGCATAGGGA ACTCAATGCA TAACTATATA ATTTGAAGAT | 1320 |
| TATAGAAGAA GGGAAATAGC AAATGGACAC AAATTACAAA TGTGTGTGCG TGGGACGAAG | 1380 |
| ACATCTTTGA AGGTCATGAG TTTGTTAGTT TAACATCATA TATTTGTAAT AGTGAAACCT | 1440 |
| GTACTCAAAA TATAAGCAGC TTGAAACTGG CTTTACCAAT CTTGAAATTT GACCACAAGT | 1500 |
| GTCTTATATA TGCAGATCTA ATGTAAAATC CAGAACTTGG ACTCCATCGT TAAAATTATT | 1560 |
| TATGTGTAAC ATTCAAATGT GTGCATTAAA TATGCTTCCA CAGTAAAATC TGAAAAACTG | 1620 |
| ATTTGTGATT GAAAGCTGCC TTTCTATTTA CTTGAGTCTT GTACATACAT ACTTTTTTAT | 1680 |
| GAGCTATGAA ATAAAACATT TTAAACTGAA TTTCTTAAAA AAAAAAAAAA A | 1731 |

### EpCAM Amino Acid Sequence Example:

| | |
|---|---|
| MAPPQVLAFG LLLAAATATF AAAQEECVCE NYKLAVNCFV NNNRQCQCTS VGAQNTVICS | 60 |
| KLAAKCLVMK AEMNGSKLGR RAKPEGALQN NDGLYDPDCD ESGLFKAKQC NGTSMCWCVN | 120 |
| TAGVRRTDKD TEITCSERVR TYWIIIELKH KAREKPYDSK SLRTALQKEI TTRYQLDPKF | 180 |
| ITSILYENNV ITIDLVQNSS QKTQNDVDIA DVAYYFEKDV KGESLFHSKK MDLTVNGEQL | 240 |
| DLDPGQTLIY YVDEKAPEFS MQGLKAGVIA VIVVVVIAVV AGIVVLVISR KKRMAKYEKA | 300 |
| EIKEMGEMHR ELNA | 314 |

An exemplary anti-EpCAM antibody is EpCAM APC clone EBA-1 from BD Biosciences (San Jose, Calif., USA), catalog #347200.

Various aspects and embodiments are described in further detail in the following subsections. A biomarker score gives an indication of the level of expression of that biomarker in a particular sample that is assayed. High levels of expression are associated with higher scores. Biomarker levels and hence scores can be obtained by methods known in the art such as ISH (e.g., to measure RNA levels) or IHC (e.g., to measure protein levels). These techniques are discussed in more detail below

### Biomarkers

The methods described herein involve one or more particular biomarkers, levels of which are measured in at least one cancer cell sample from a patient.

Scores for any single one of the biomarkers c-Met, HGF, and EpCAM can be used in the methods provided herein. In the case of c-Met, high level expression (scores of 2+, or more definitively 3+) may result in ligand-independent activation of c-Met (e.g., via autophosphorylation, e.g., as a result of concentration-driven homodimerization of c-Met in c-Met overexpressing cancer cells) regardless of the presence of absence of a ligand such as HGF. This, taken together with the high level of binding of anti-c-Met/anti-EpCAM bispecific antibodies to cells overexpressing c-Met (even in the absence of EpCAM), renders c-Met a particularly useful single biomarker for anti-c-Met/anti-EpCAM bispecific antibodies that block ligand-independent activation of c-Met in cells overexpressing c-Met. Therefore patients who express high levels of c-Met (e.g., as determined on the basis of the biomarker) will benefit from treatment with anti-c-Met/anti-EpCAM bispecific antibodies that block ligand-independent activation of c-Met in cells overexpressing c-Met.

Additionally, scores for each of any combination of the biomarkers described herein can be used. In one embodiment, the scores of at least two biomarkers are used (e.g., c-Met and HGF; c-Met and EpCAM; c-Met, HGF, and EpCAM; EpCAM and HGF).

### Biopsy Samples

The expression of one or more biomarkers may be determined in a biopsy sample (biopsy) obtained from a subject. Such a sample is typically further processed after it is obtained from the subject. Biopsy samples suitable for detecting and quantitating the biomarkers described herein may be fresh, frozen, or fixed. Suitable samples are preferably sectioned. Alternatively, samples may be solubilized and/or homogenized and subsequently analyzed.

In one embodiment, a freshly obtained biopsy sample is embedded in a cryoprotectant such as OCT® or Cryomatrix® and frozen using, for example, liquid nitrogen or difluorodichloromethane. The frozen sample is serially sectioned in a cryostat. In another embodiment, samples are fixed and embedded prior to sectioning. For example, a tissue sample may be fixed in, for example, formalin, glutaraldehyde, ethanol or methanol, serially dehydrated (e.g., using alcohol and or xylenes) and embedded in, for example, paraffin.

In one embodiment, the sample is prepared as a microtome section of a biopsy (e.g., FFPE prior to microtome sectioning). In another embodiment, the biopsy is obtained within 30, 60, or 90 days prior to treating the patient. In certain embodiments, the methods of the invention include one or more of the steps required to process the sample from the subject, including (a) fixing or freezing the sample, (b) sectioning the sample, (c) solubilizing and/or homogenizing the sample. In yet some embodiments, the methods of the invention further comprise the step of obtaining the sample from the patient. In other embodiments, the methods are carried out on samples which have been obtained from the patient.

### Circulating Tumor Cell Samples

Circulating tumor cells are cells that have detached from a primary tumor and entered the vascular system. These may be found in frequencies on the order of 1-10 CTC per mL of whole blood in patients with metastatic disease and the isolation of these cells may offer a non-invasive alternative to tumor biopsies and may often be used in cases where a procuring a biopsy sample isn't possible.

### Detecting and Scoring Biomarkers

### Nucleic Acid Assays

In various embodiments, expression of the biomarker is detected at the nucleic acid level. For example, the biomarker score for HGF can be assessed based on HGF RNA levels. In one embodiment, RNA is detected using an RNA-ISH assay as discussed in further detail below.

Another method for determining the level of RNA in a sample involves the process of nucleic acid amplification from homogenized tissue, e.g., by RT-PCR (reverse transcribing the RNA and then, amplifying the resulting cDNA employing PCR or any other nucleic acid amplification method, followed by the detection of the amplified molecules.

In particular aspects, RNA expression is assessed by quantitative fluorogenic RT-PCR (qPCR) e.g., by using the TaqMan™ System. Such methods typically utilize pairs of oligonucleotide primers that are specific for the nucleic acid of interest. Further details of such assays are provided below in the Examples. A suitable ordinal scoring system is shown below when mRNA levels are measured in FFPE tissue sections:

| Staining | |
|---|---|
| Score | Microscope objective scoring* |
| 0 | No staining or less than 1 dot per 10 cells (40x magnification) |
| 1 | 1-3 dots/cell (visible at 20-40x magnification) |
| 2 | 4-10 dots/cell. Very few dot clusters (visible at 20-40x |
| | magnification) |
| 3 | >10 dots/cell. Fewer than 10% positive cells have dot clusters |
| | (visible at 20x magnification) |
| 4 | >10 dots/cell. Greater than 10% positive cells have dot clusters |
| | (visible at 20x magnification) |

*If <5% of cells score 1 and >95% of cells score 0, a score of 0 will be given.
If 5-30% of cells score 1 and >70% of cells score 0, a score of 0.5 will be given.
Scoring performed at 20x magnification.

### Protein Assays

Expression of the biomarker also can be detected at the protein level. Accordingly, the score for c-Met, EpCAM, or HGF can be assessed based on detected levels of protein. In a particular embodiment, expression of protein levels is measured using immunohistochemistry (IHC). Immunohistochemistry is a technique for detecting proteins in cells of a tissue section by using antibodies that specifically bind to the proteins. Exemplary IHC assays, such as Fl-IHC and qIHC are described in further detail below.

Exemplary IHC assays, such as Fl-IHC and qIHC are described in further detail below in the Examples.

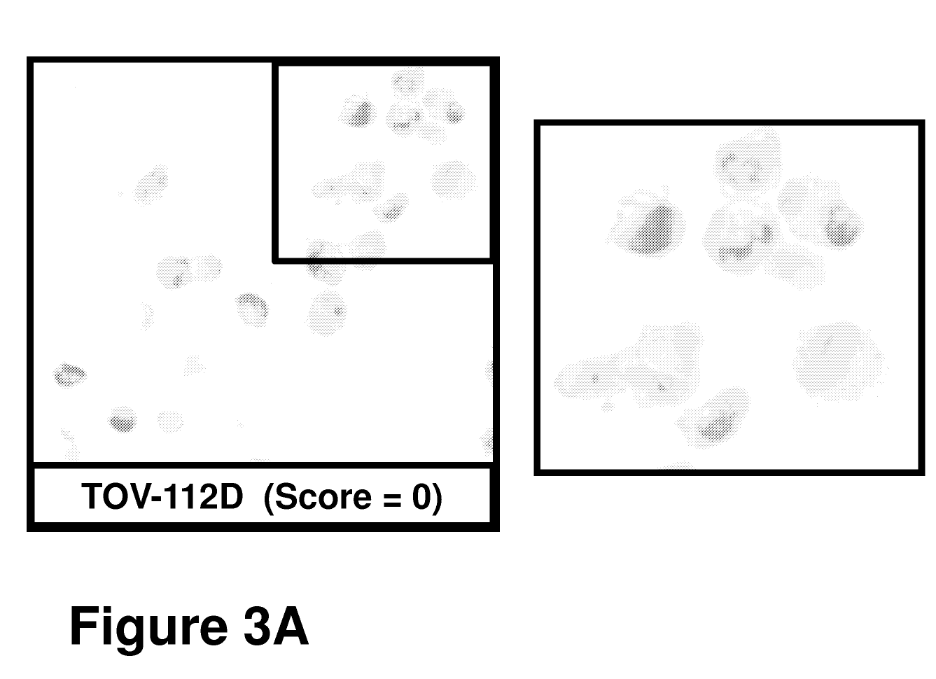

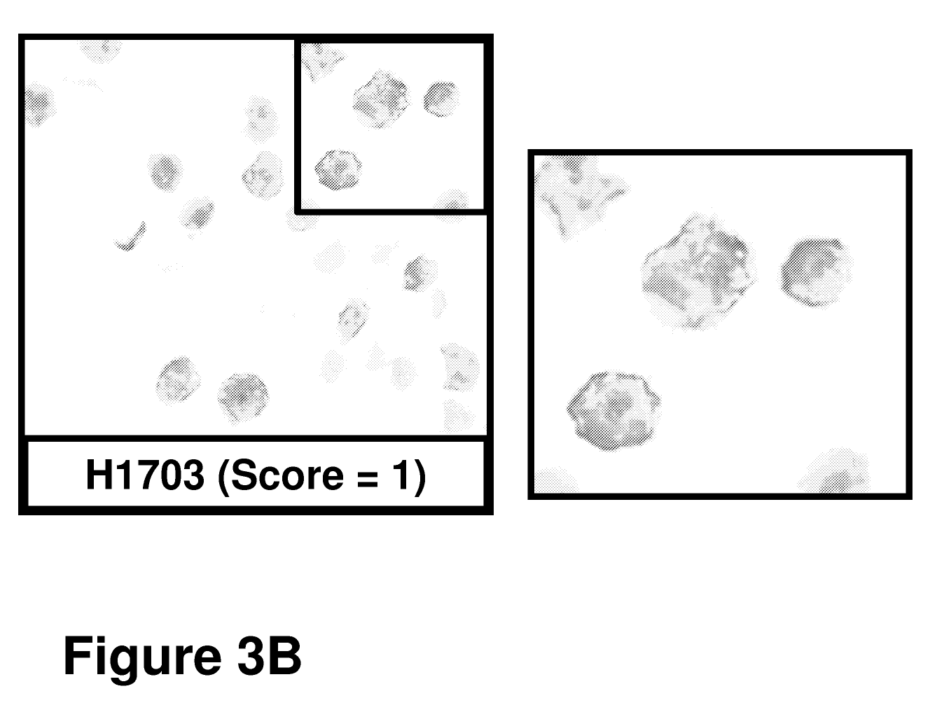

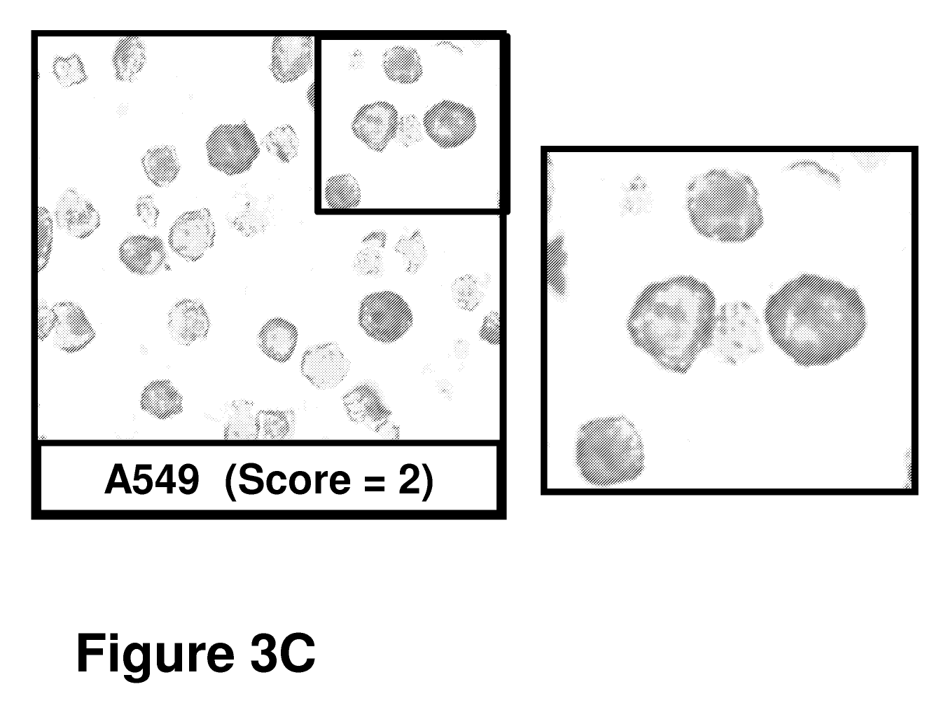

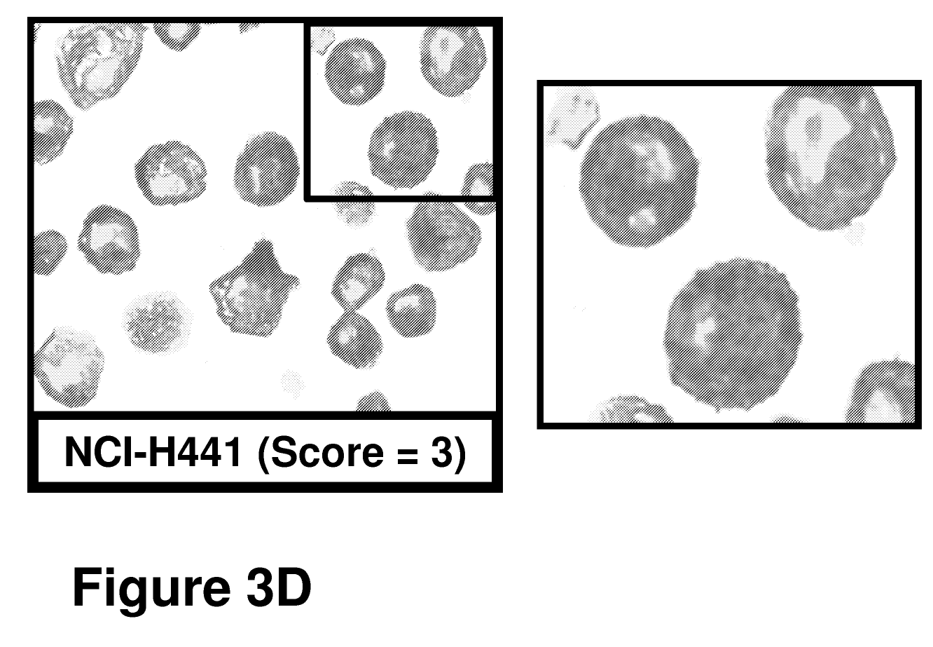

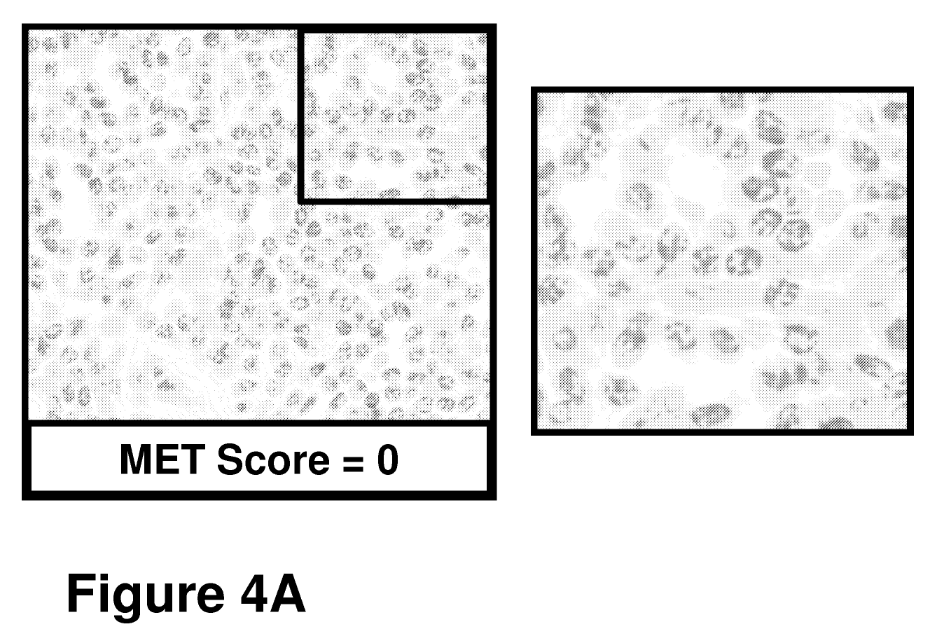

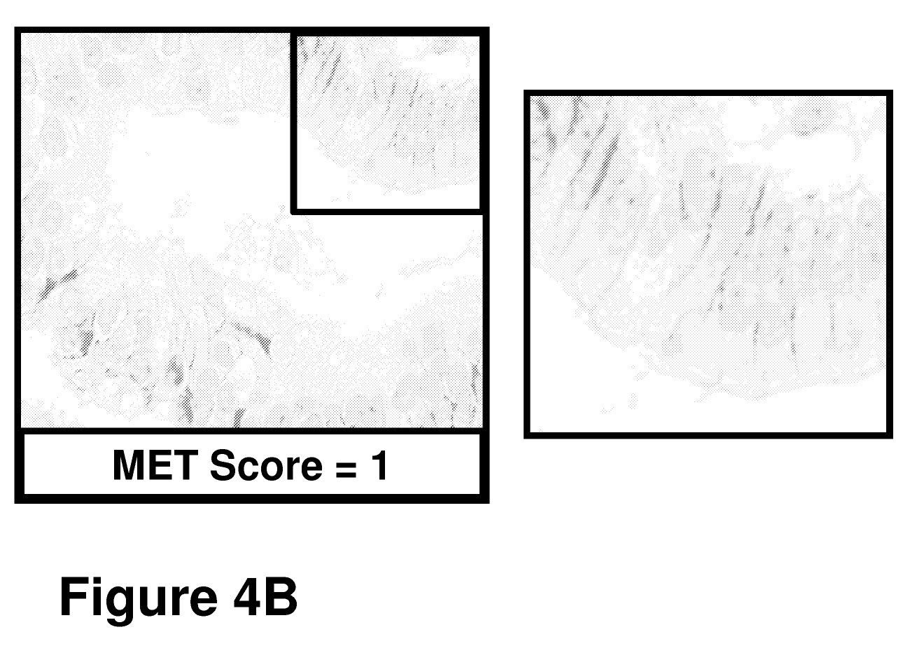

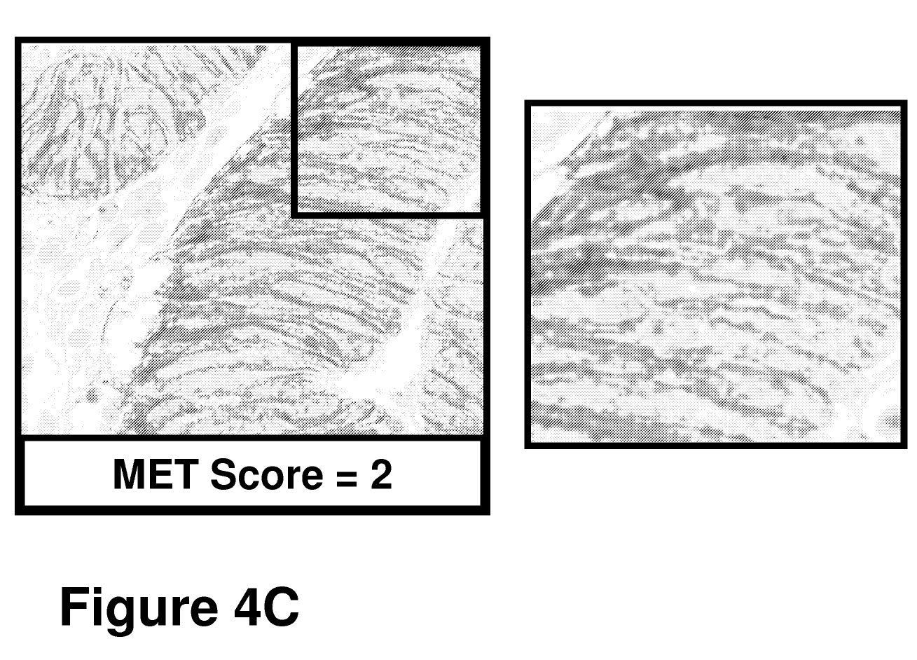

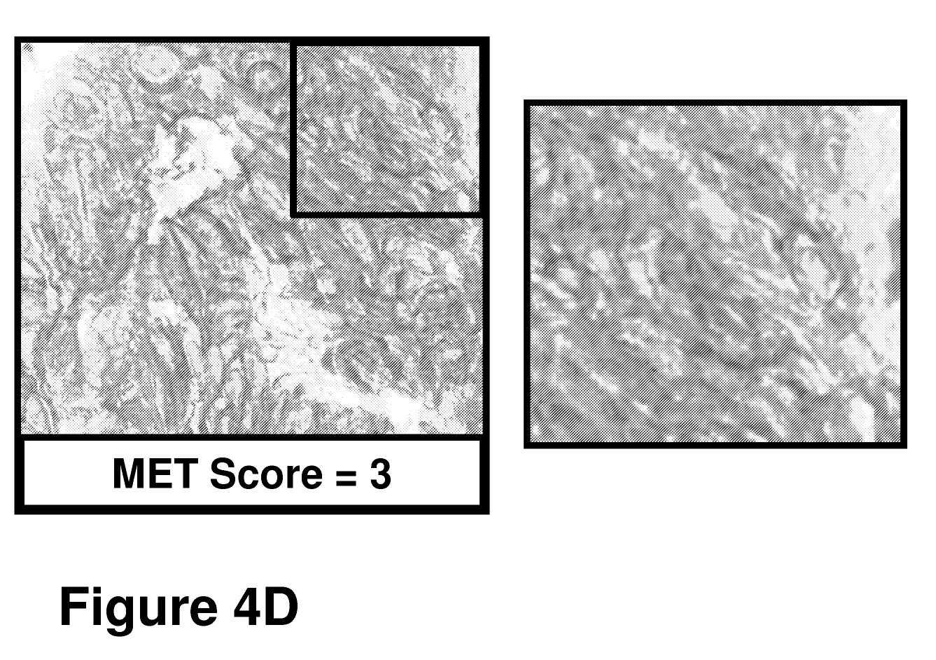

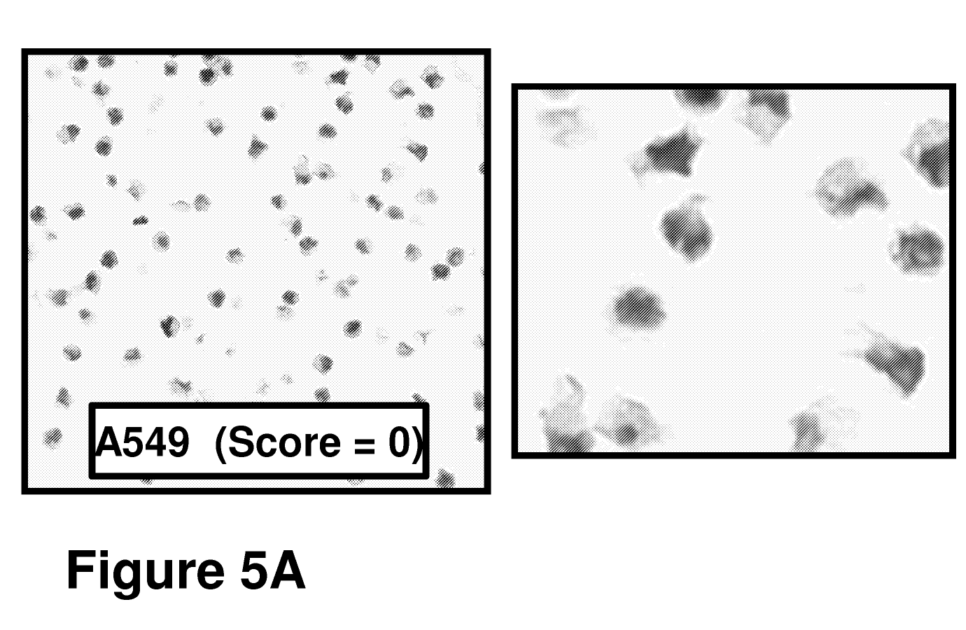

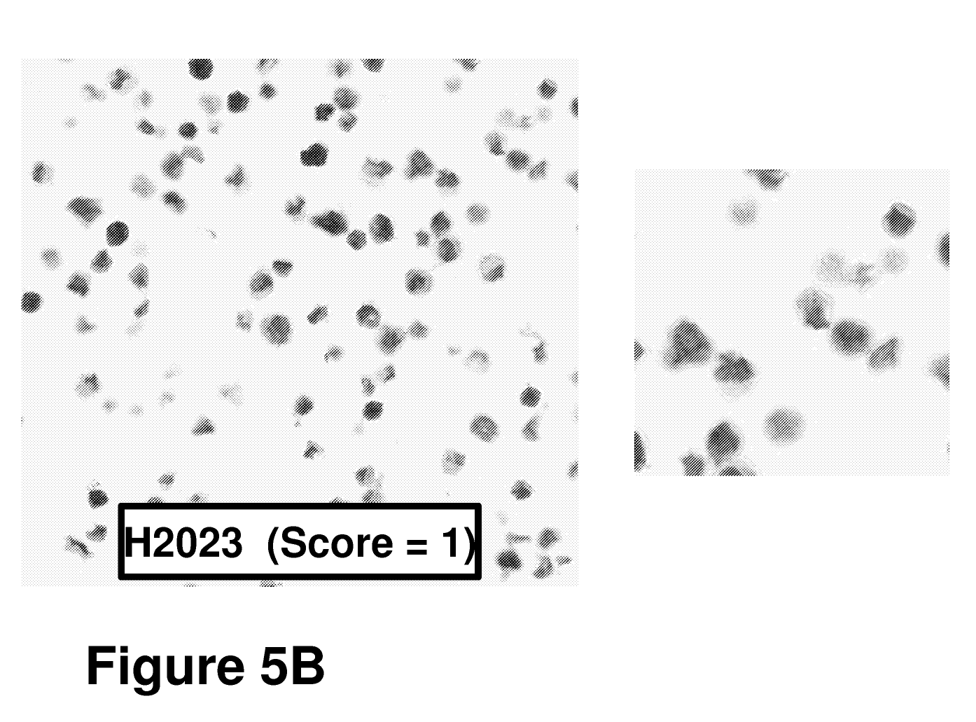

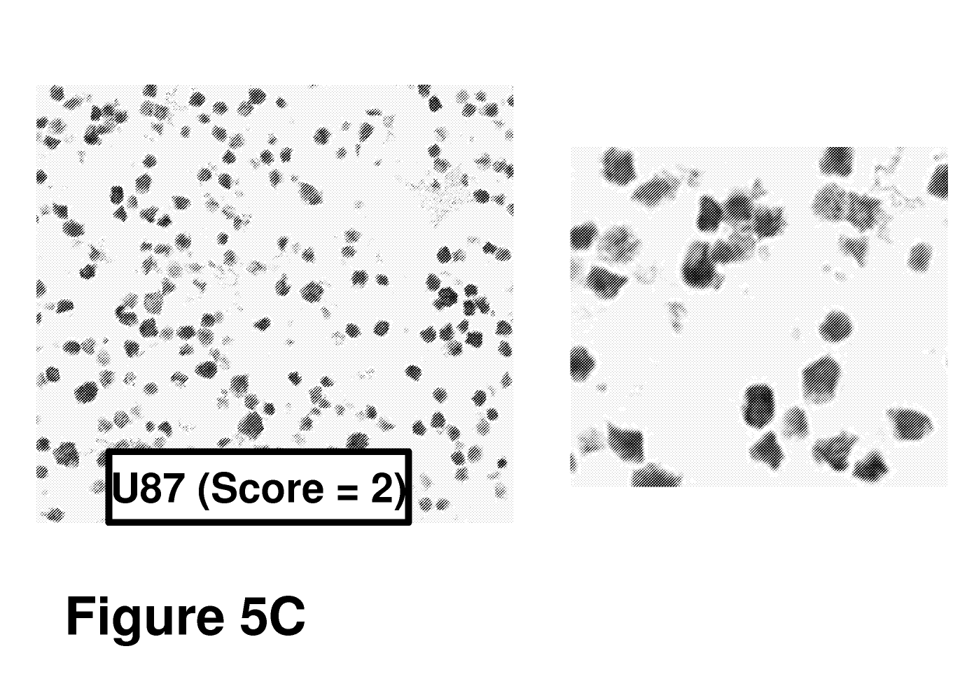

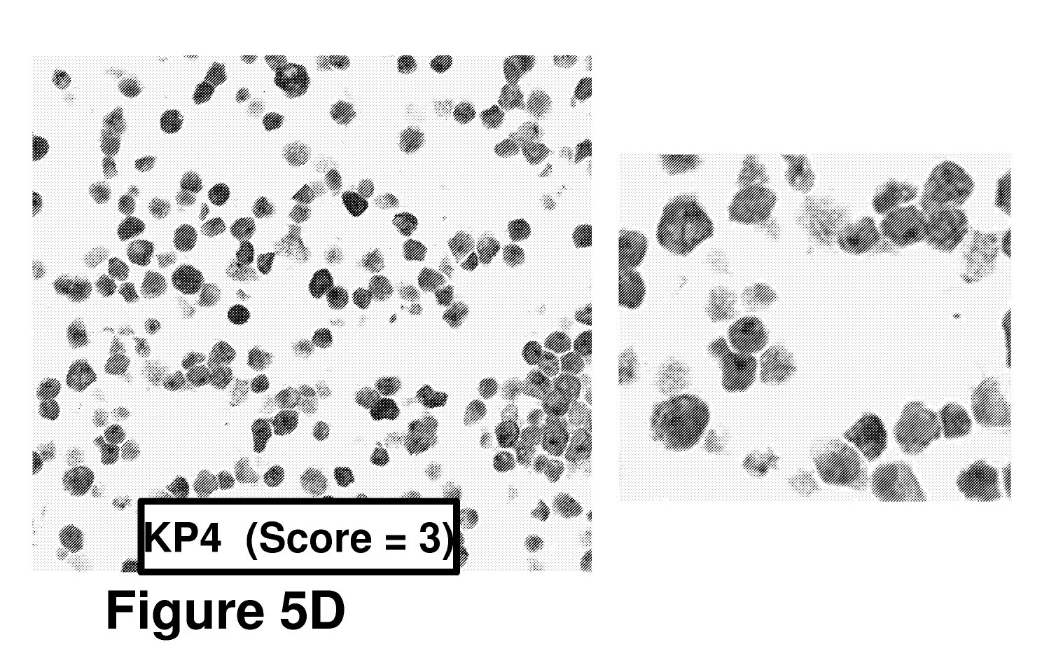

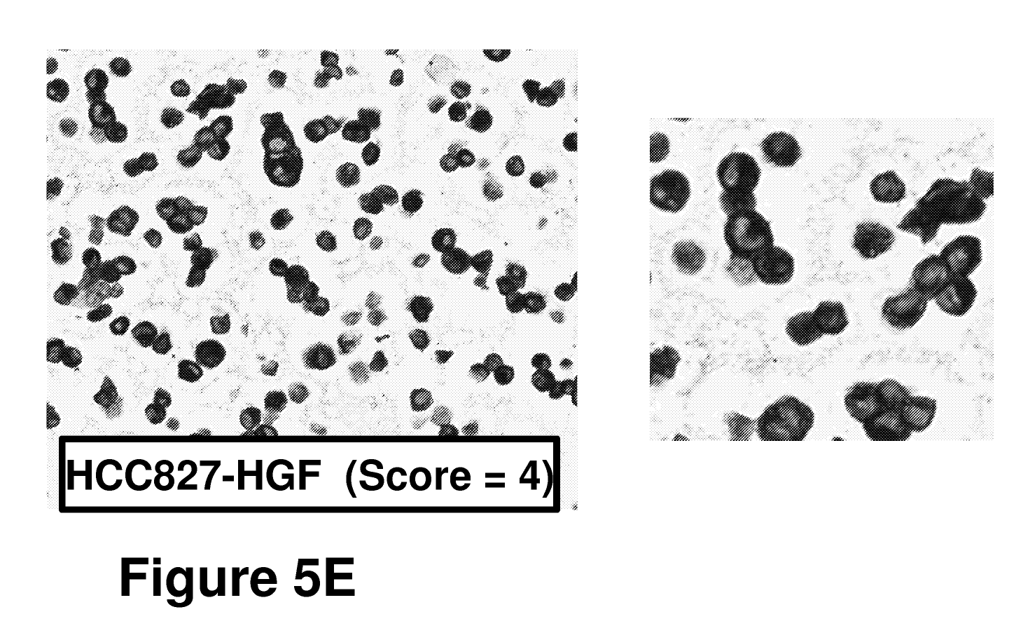

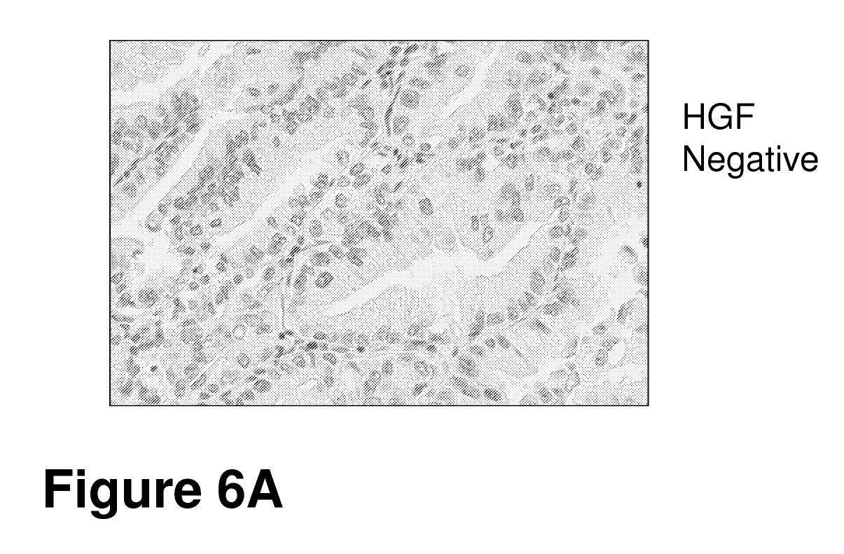

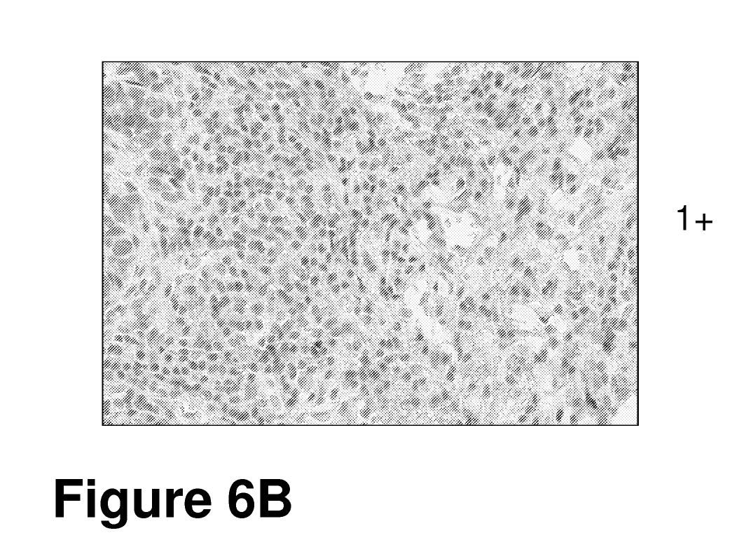

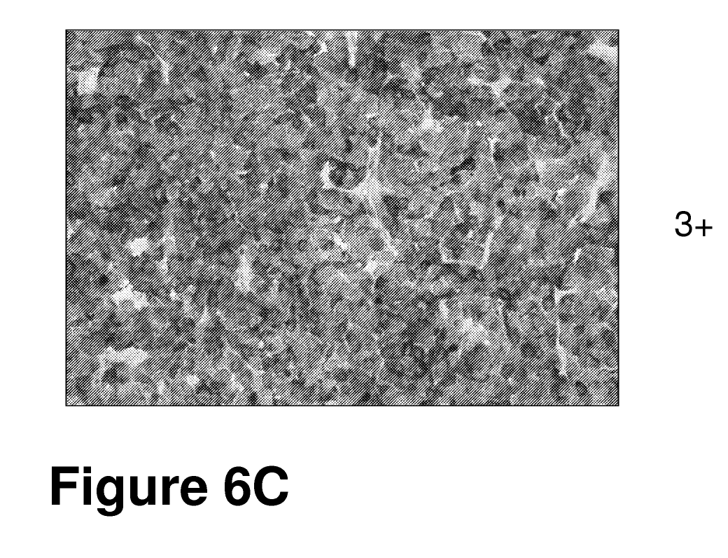

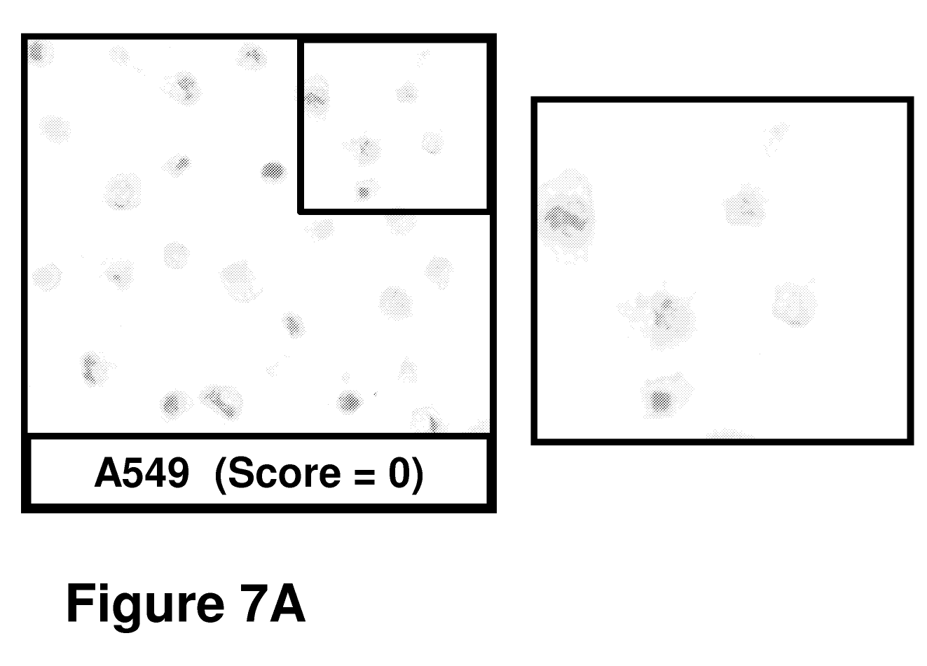

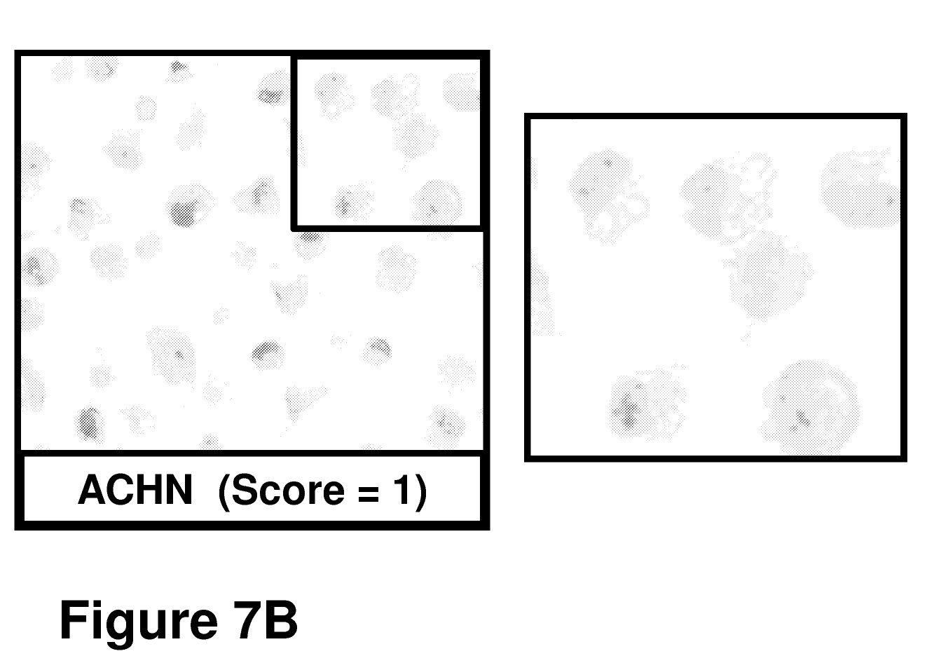

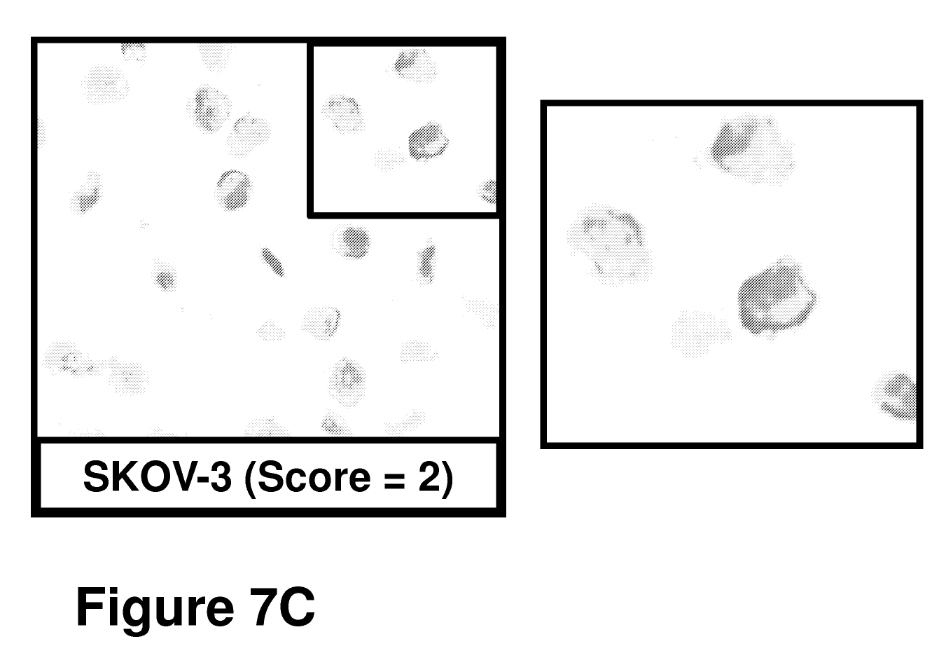

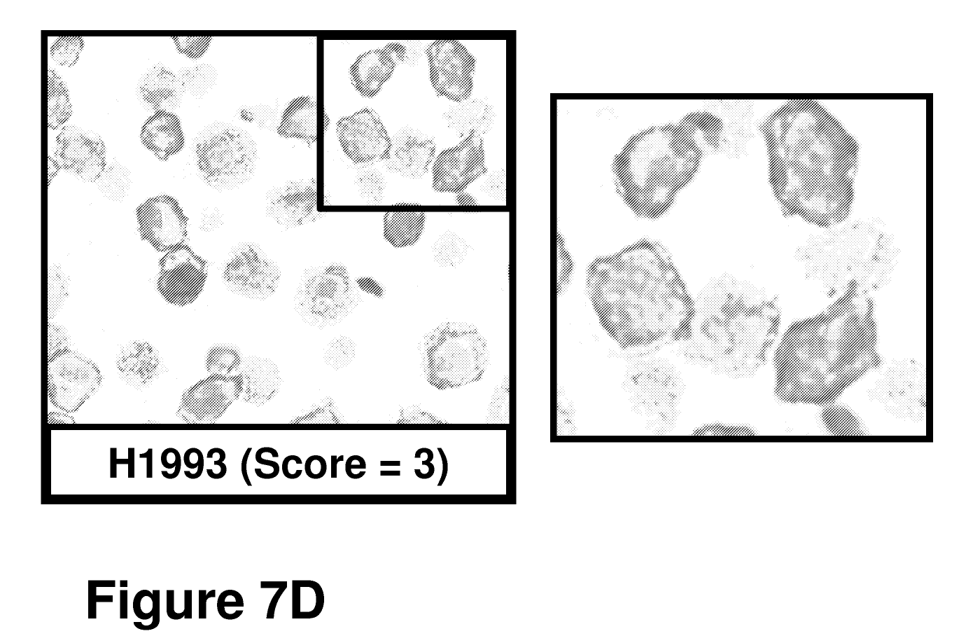

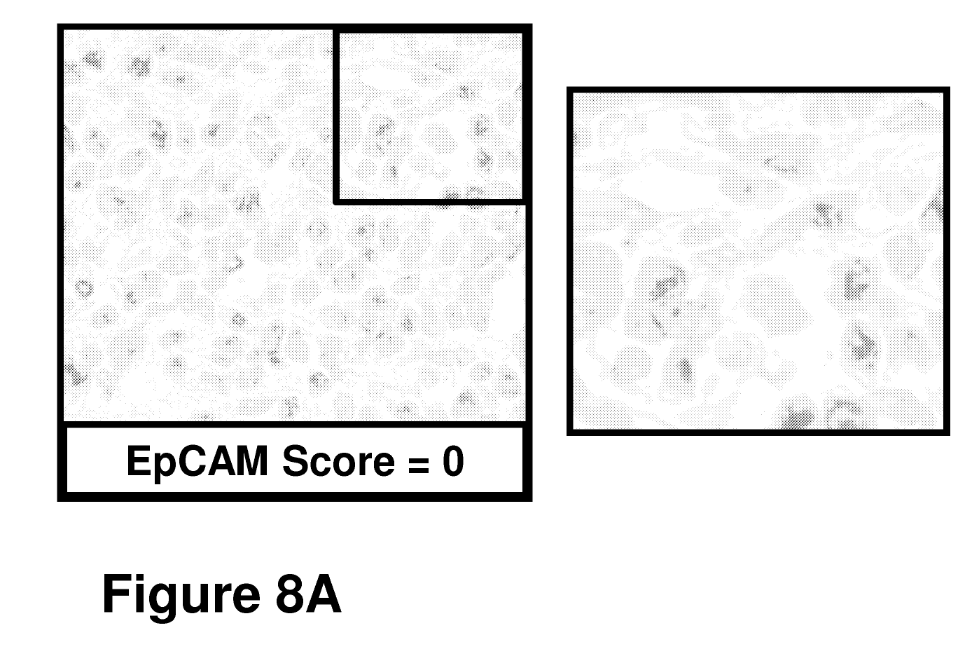

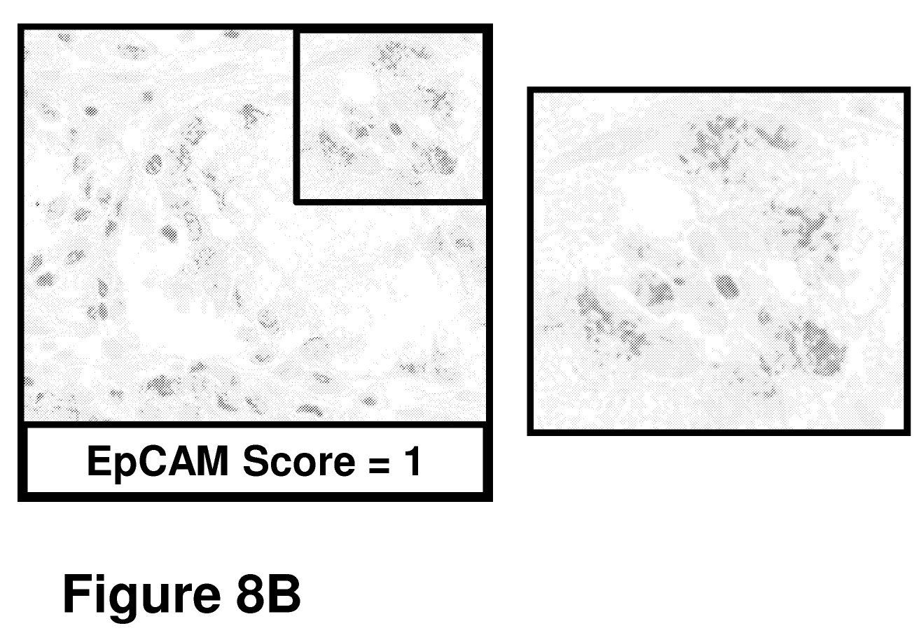

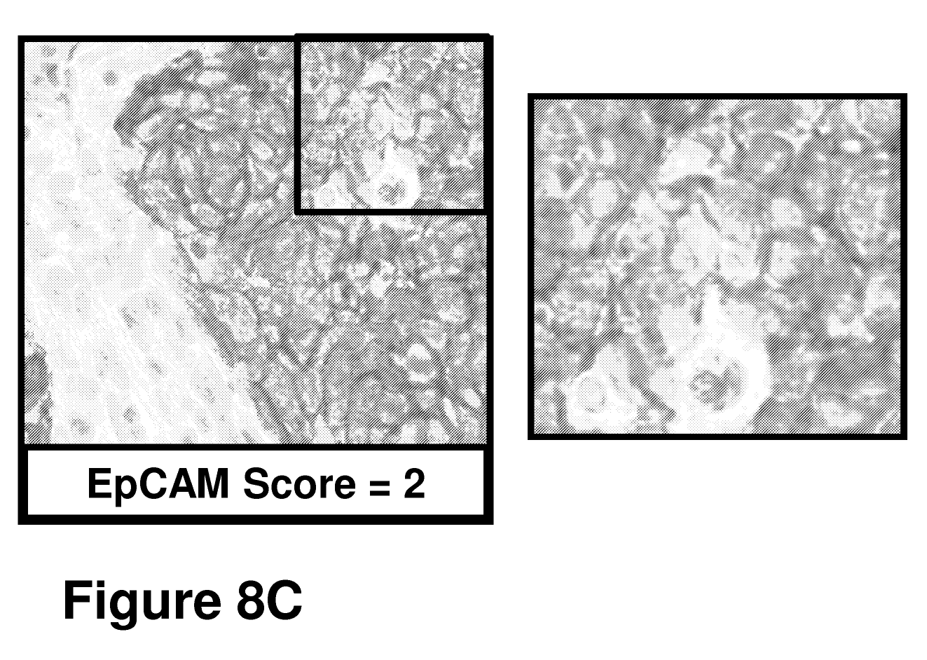

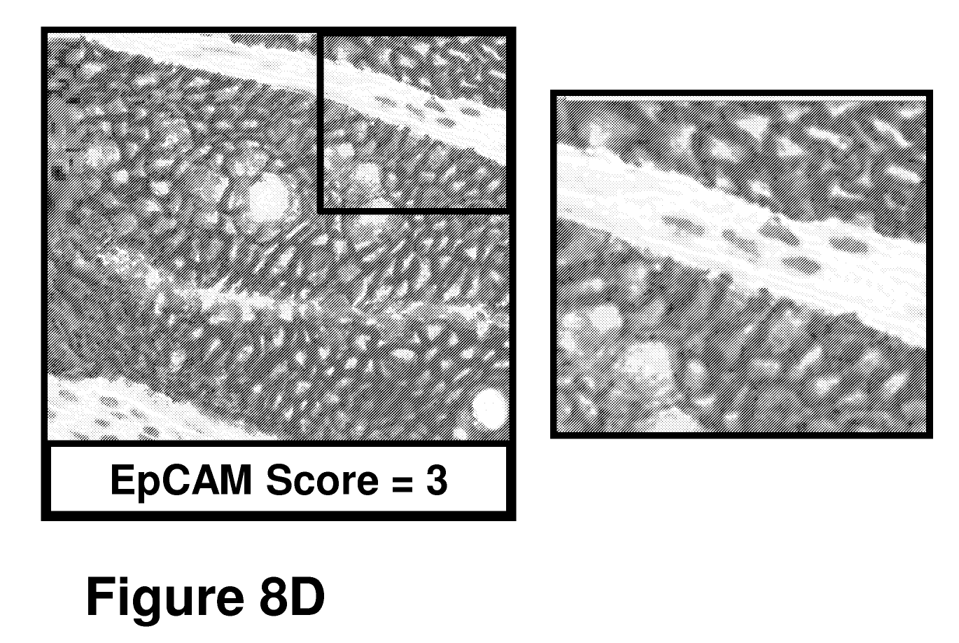

Scoring of a sample stained for the detection of protein levels is performed using image analysis software capable of determining the number of staining intensity in a cell or by a trained pathologist who is familiar with the pattern created when the chromogen precipitates at the site of antibody binding in the case of tissue that has been stained with protein-specific antibodies using a chromogen as a detectable agent. A suitable system uses a system rating from no staining to high staining, 0, 1, 2, 3, and 4. Examples of these levels of staining using the protocols set forth in Example 4 are shown in FIGS. 3 and 4 (for anti-c-Met staining), FIGS. 5 and 6 (for anti-HGF staining), and FIGS. 7 and 8 (for anti-EpCAM staining).

c-Met Inhibitors

Methods provided herein can be used to predict efficacy of therapeutic treatment using any suitable c-Met inhibitor or combination of inhibitors, either alone (e.g., as monotherapy) or in combination with other therapeutic agents.

In one embodiment, the c-Met inhibitor is a bispecific anti-c-Met, anti-EpCAM antibody. Exemplary bispecific anti-c-Met, anti-EpCAM antibodies are described, e.g., in co-pending U.S. Patent Publication No. 2014-0294834. The general structure of the bispecific antibodies disclosed herein comprises a bivalent antibody with a single Fab directed against c-Met, a TFc backbone structure (described herein and in copending PCT Application Serial No. PCT/US2012/52490, e.g., SEQ ID NO:394 and 395), and a single scFv antibody fragment directed against EpCAM.

In one embodiment, the c-Met inhibitor is an anti-c-Met, anti-EpCAM TFcA, which may be monovalent or polyvalent, e.g., bivalent, trivalent, or tetravalent. TFcAs which are polyvalent may be monospecific, bispecific, trispecific, or tetraspecific. When a TFcBA is multispecific, it may be monovalent for one or more specificities.

In certain embodiments, the TFcBA comprises a first binding site (e.g., an anti-c-Met Fab), a second binding site (e.g., an anti-EpCAM scFv), and a TFc that links the first and the second binding sites together. A TFcBA may be described as containing three modules, wherein the first module comprises the first binding site, the second module comprises the TFc and the third module comprises the second binding site. A TFc generally comprises in a contiguous amino acid sequence a first Fc region, a TFc linker, and a second Fc region, wherein the TFc linker links the first Fc region to the second Fc region and allows the association of the two Fc regions. Each of the two Fc regions of a TFc may comprise a hinge, a CH2 domain and a CH3 domain. Each of these regions may be from the same immunoglobulin isotype, or from different isotypes. For example, the hinge, CH2 and CH3 domains may all be from IgG1, IgG2, IgG3 or IgG4, or certain domains or portions thereof may be from one immunoglobulin isotype and another domain or portion may be from another immunoglobulin isotype. For example, a TFcBA may comprise all domains from IgG1, or alternatively, it may comprise an IgG1/IgG4 hybrid hinge, an IgG4 CH2 domain and an IgG1 CH3 domain. An Fc region preferably comprises human Fc domains, however, sequences from other mammals or animals may also be used, provided that the TFcBA retains its biological activity and is preferably not significantly immunogenic in a human subject.

Exemplary TFcBAs inhibit ligand-induced signal transduction through one or both of the receptors targeted by the TFcBA and may thereby inhibit tumor cell proliferation or tumor growth. TFcBAs may also induce receptor downregulation or block receptor dimerization. Exemplary anti c-Met/EpCAM TFcBAs comprise a single anti-c-Met binding site (monovalent for anti-c-Met) and one or more anti-EpCAM binding sites (monovalent or polyvalent for anti-EpCAM). Nucleic acid and amino acid sequences for exemplary bispecific anti-c-Met, anti-EpCAM antibodies are set forth below.

```
Sequence information for additional embodiments
Amino Acid (aa) and Nucleotide (nt) sequences
Underline: CDRs
Bold sequence: Stabilization mutations
>anti-c-Met-LC aa
(SEQ ID NO: 400)
(Light Chain for SEQ ID NOs: 403-415)
DIQMTQSPSSVSASVGDRVTITCRASQGISSWLAWYQQKPGKAPKLLIYAASSLQSGVPSRFSGSGSGTDFTLTI
SS
                                                        CDR1 CDR2
LQPEDFATYYCLQANSFPPTFGGGTKVEIKRTVAAPSVFIFPPSDEQLKSGTASVVCLLNNFYPREAKVQWKVDN
AL
                          CDR3
QSGNSQESVTEQDSKDSTYSLSSTLTLSKADYEKHKVYACEVTHQGLSSPVTKSFNRGEC**
>RESERVED SEQUENCE
(SEQ ID NO: 401)
XXXX
>anti-c-Met-LC nt
(SEQ ID NO: 402)
(Light Chain for SEQ ID NOs: 403-415)
gacattcagatgacccagtctccttcaagcgtcagcgcttccgtgggcgaccgggtcaccatcacatgcagagcc
tcccaggggattagctcctggctggcttggtatcagcagaagcctgggaaagcaccaaagctgctgatctatgcc
gcttctagtctgcagtccggagtgccctctcgattctctggcagtgggtcaggaaccgactttactctgaccatt
tcaagcctgcagcctgaggatttcgctacatactattgcctgcaggcaaactctttcccccctacctttggcggg
ggaacaaaagtggagatcaagcgtacggtggcagccccatccgtcttcatttttccaccctctgacgaacagctg
aaaagtggcacagccagcgtggtctgtctgctgaacaatttttacccccgcgaagccaaagtgcagtggaaggtc
gataacgctctgcagagcgggaattcccaggagtctgtgactgaacaggacagtaaagattcaacctatagcctg
tcctctacactgactctgagcaaggcagattacgagaagcacaaagtgtatgcctgcgaagtcacacatcaggga
ctgagttcacctgtgactaagagcttcaatagaggcgagtgttgataa
>Ab #1-HC-aa
(SEQ ID NO: 403)
QLQLQESGPGLVKPSETLSLTCTVSGGSISSSVYYWSWIRQPPGKGLEWIGVIYPSGNTYYSPSLKSRVTISVDT
SKNQFSLKLSSVTAADTAVYYCARTIYDLFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPSCPAPEFLGGPSVFLFPPKPKDTLMISRTPEVTCVVVDVSQEDPEVQFNWYVDGVEVHNAKTKPREEQFNSK
YRVVSVLTVLHQDWLNGKEYKCKVSNKGLPSSIEKTISKAKGQPREPQVYTLPPSREEMTKNQVSLSCAVKGFYP
SDIAVEWESNGQPENNYKTTPPVLDSDGSFFLVSKLTVDKSRWQQGNVFSCSVMHEALHNHYTQKSLSLSKSCDK
TGGGGSGGGGSGGGGSGGGGSGGGGSGGGGSGGGGSGGGGSCPSCPAPEFLGGPSVFLFPPKPKDTLMISRTPEV
TCVVVDVSQEDPEVQFNWYVDGVEVHNAKTKPREEQFNSDYRVVSVLTVLHQDWLNGKEYKCKVSNKGLPSSIEK
TISKAKGQPREPQVYTLPPSREEMTKNQVSLWCLVKGFYPSDIAVEWESNGQPENNYKTTPPVLDSDGSFFLYSK
LTVDKSRWQQGNVFSCSVMHEALHNHYTQKSLSLSGECGGGGSGGGGS
DIVMTQSPLSLPVTPGEPASISCRSTKSLLHSNGITYLYWYLQKPGQSPQLLIYQMSNLASGVPDRFSSSGSGTD
FTLKISRVEAEDEGVYYCAQNLEIPRTFGGGTKLEIKRTGGGGSGGGGSGGGGSGGGGSQVQLVQSGAEVKKPGE
SVKISCKASGYTFTNYGMNWVRQAPGQGLKWMGWINTYTGESTYADDFKGRFAFSLDTSASTAYLQLSSLRSEDT
AVYFCARFAIKGDYWGQGTLVTVSS**
>Ab #2-HC-aa
(SEQ ID NO: 404)
QLQLQESGPGLVKPSETLSLTCTVSGGSISSSVYYWSWIRQPPGKGLEWIGVIYPSGNTYYSPSLKSRVTISVDT
SKNQFSLKLSSVTAADTAVYYCARTIYDLFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPSCPAPEFLGGPSVFLFPPKPKDTLMISRTPEVTCVVVDVSQEDPEVQFNWYVDGVEVHNAKTKPREEQFNSK
YRVVSVLTVLHQDWLNGKEYKCKVSNKGLPSSIEKTISKAKGQPREPQVYTLPPSREEMTKNQVSLSCAVKGFYP
SDIAVEWESNGQPENNYKTTPPVLDSDGSFFLVSKLTVDKSRWQQGNVFSCSVMHEALHNHYTQKSLSLSKSCDK
TGGGGSGGGGSGGGGSGGGGSGGGGSGGGGSGGGGSGGGGSCPSCPAPEFLGGPSVFLFPPKPKDTLMISRTPEV
TCVVVDVSQEDPEVQFNWYVDGVEVHNAKTKPREEQFNSDYRVVSVLTVLHQDWLNGKEYKCKVSNKGLPSSIEK
TISKAKGQPREPQVYTLPPSREEMTKNQVSLWCLVKGFYPSDIAVEWESNGQPENNYKTTPPVLDSDGSFFLYSK
LTVDKSRWQQGNVFSCSVMHEALHNHYTQKSLSLSGECGGGGSGGGGS
QVQLVQSGSELKKPGESVKVSCKASGYTFTNYGMHWVRQAPGQGLKWMGWINTYTGESTYADDFKGRFAFSLDTS
ASTAYLQISSLKAEDTAVYFCARFAIKGDYWGQGTLVTVSSASTGGGGSGGGGSGGGGSGGGGSDIVMTQSPLSL
PVTPGEPASISCRSTKSLLHSDGITYLYWYLQKPGQSPQLLIYQLSNLASGVPDRFSSSGSGTDFTLKISRVEAE
DEGVYYCAQNLEIPYTFGQGTKVEIK**
>Ab #3-HC-aa
(SEQ ID NO: 405)
QLQLQESGPGLVKPSETLSLTCTVSGGSISSSVYYWSWIRQPPGKGLEWIGVIYPSGNTYYSPSLKSRVTISVDT
SKNQFSLKLSSVTAADTAVYYCARTIYDLFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPSCPAPEFLGGPSVFLFPPKPKDTLMISRTPEVTCVVVDVSQEDPEVQFNWYVDGVEVHNAKTKPREEQFNSK
YRVVSVLTVLHQDWLNGKEYKCKVSNKGLPSSIEKTISKAKGQPREPQVYTLPPSREEMTKNQVSLSCAVKGFYP
SDIAVEWESNGQPENNYKTTPPVLDSDGSFFLVSKLTVDKSRWQQGNVFSCSVMHEALHNHYTQKSLSLSKSCDK
TGGGGSGGGGSGGGGSGGGGSGGGGSGGGGSGGGGSGGGGSCPSCPAPEFLGGPSVFLFPPKPKDTLMISRTPEV
TCVVVDVSQEDPEVQFNWYVDGVEVHNAKTKPREEQFNSDYRVVSVLTVLHQDWLNGKEYKCKVSNKGLPSSIEK
TISKAKGQPREPQVYTLPPSREEMTKNQVSLWCLVKGFYPSDIAVEWESNGQPENNYKTTPPVLDSDGSFFLYSK
LTVDKSRWQQGNVFSCSVMHEALHNHYTQKSLSLSGECGGGGSGGGGS
QVQLVQSGSELKKPGESVKVSCKASGYTFTNYGMNWVRQAPGQGLKWMGWINTYTGESTYADDFKGRFAFSLDTS
ASTAYLQISSLKAEDTAVYFCARFAIKGDYWGQGTLVTVSSASTGGGGSGGGGSGGGGSGGGGSDIVMTQSPLSL
PVTPGEPASISCRSTKSLLHSDGITYLYWYLQKPGQSPQLLIYQLSNLASGVPDRFSSSGSGTDFTLKISRVEAE
DEGVYYCAQNLEIPRTFGQGTKVEIK**
>Ab #4-HC-aa
(SEQ ID NO: 406)
QLQLQESGPGLVKPSETLSLTCTVSGGSISSSVYYWSWIRQPPGKGLEWIGVIYPSGNTYYSPSLKSRVTISVDT
SKNQFSLKLSSVTAADTAVYYCARTIYDLFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPSCPAPEFLGGPSVFLFPPKPKDTLMISRTPEVTCVVVDVSQEDPEVQFNWYVDGVEVHNAKTKPREEQFNSK
YRVVSVLTVLHQDWLNGKEYKCKVSNKGLPSSIEKTISKAKGQPREPQVYTLPPSREEMTKNQVSLSCAVKGFYP
SDIAVEWESNGQPENNYKTTPPVLDSDGSFFLVSKLTVDKSRWQQGNVFSCSVMHEALHNHYTQKSLSLSKSCDK
TGGGGSGGGGSGGGGSGGGGSGGGGSGGGGSGGGGSGGGGSCPSCPAPEFLGGPSVFLFPPKPKDTLMISRTPEV
TCVVVDVSQEDPEVQFNWYVDGVEVHNAKTKPREEQFNSDYRVVSVLTVLHQDWLNGKEYKCKVSNKGLPSSIEK
TISKAKGQPREPQVYTLPPSREEMTKNQVSLWCLVKGFYPSDIAVEWESNGQPENNYKTTPPVLDSDGSFFLYSK
LTVDKSRWQQGNVFSCSVMHEALHNHYTQKSLSLSGECGGGGSGGGGS
DIVMTQSPLSLPVTPGEPASISCRSTKSLLHSDGITYLYWYLQKPGQSPQLLIYQLSNLASGVPDRFSSSGSGTD
FTLKISRVEAEDEGVYYCAQNLEIPRTFGQGTKVEIKRTGGGGSGGGGSGGGGSGGGGSGQVQLVQSGSELKKPG
ESVKVSCKASGYTFTNYGMNWVRQAPGQGLKWMGWINTYTGESTYADDFKGRFAFSLDTSASTAYLQISSLKAED
TAVYFCARFAIKGDYWGQGTLVTVSS**
>Ab #5-HC-aa
(SEQ ID NO: 407)
QLQLQESGPGLVKPSETLSLTCTVSGGSISSSVYYWSWIRQPPGKGLEWIGVIYPSGNTYYSPSLKSRVTISVDT
SK
                                                              CDR1 CDR2
NQFSLKLSSVTAADTAVYYCARTIYDLFDIWGQGTMVTVSSASTKGPSVFPLAPSSKSTSGGTAALGCLVKDYFP
EP
                                                  CDR3
VTVSWNSGALTSGVHTFPAVLQSSGLYSLSSVVTVPSSSLGTQTYICNVNHKPSNTKVDKKVEPKSCDKTHTCPS
CPAPEFLGGPSVFLFPPKPKDTLMISRTPEVTCVVVDVSQEDPEVQFNWYVDGVEVHNAKTKPREEQFNSKYRVV
SVLTVLHQDWLNGKEYKCKVSNKGLPSSIEKTISKAKGQPREPQVYTLPPSREEMTKNQVSLSCAVKGFYPSDIA
VEWESNGQPENNYKTTPPVLDSDGSFFLVSKLTVDKSRWQQGNVFSCSVMHEALHNHYTQKSLSLSKSCDKTGGG
GSGGGGSGGGGSGGGGSGGGGSGGGGSGGGGSGGGGSCPSCPAPEFLGGPSVFLFPPKPKDTLMISRTPEVTCVV
VDVSQEDPEVQFNWYVDGVEVHNAKTKPREEQFNSDYRVVSVLTVLHQDWLNGKEYKCKVSNKGLPSSIEKTISK
AKGQPREPQVYTLPPSREEMTKNQVSLWCLVKGFYPSDIAVEWESNGQPENNYKTTPPVLDSDGSFFLYSKLTVD
KSRWQQGNVFSCSVMHEALHNHYTQKSLSLSGECGGGGSGGGGSDIVMTQSPLSLPVTPGEPASISCRSTKSLLH
SNGITYLYWYLQKPGQ
                                                                                                                        CDR4
SPQLLIYQMSNLASGVPDRFSSSGSGTDFTLKISRVEAEDEGVYYCAQNLEIPRTFGGGTKLEIKRTGGGGSGGG
GS
                  CDR5 CDR6
GGGGSGGGGSQVQLVQSGAEVKKPGESVKISCKASGYTFTNYGMNWVRQAPGQGLKWMGWINTYTGESTYADDFK
GR
                                                                              CDR7 CDR8
FAFSLDTSASTAYLQLSSLRSEDTAVYFCARFAIKGDYWGQGTLVTVSS**
                                                                    CDR9
>Ab #5-HC-nt
(SEQ ID NO: 408)
cagctgcagctgcaggagagcggacccggactggtcaagccctccgagacactgtctctgacctgcacagtgtca
ggcgggagcatcagctcctctgtctactattggtcttggattcggcagccccctggaaagggcctggaatggatc
ggcgtgatctacccaagtgggaacacttactattctcccagtctgaaatccagagtgaccatcagtgtcgacaca
tcaaagaatcagttcagcctgaaactgagttcagtgacagccgctgatactgcagtctactattgtgccaggacc
atctatgacctgtttgatatttgggggcagggaactatggtgaccgtcagctccgctagcactaagggaccttcc
gtgttcccactggccccctcaagcaaatctaccagtgggggaacagcagccctgggctgtctggtgaaggattac
tttccagagcccgtgaccgtcagttggaactcaggggctctgactagcggagtgcacaccttccccgcagtcctg
cagtcctctgggctgtactccctgagttcagtggtcactgtgcccagctcctctctgggaacacagacttatatc
tgcaacgtgaatcacaagccttccaataccaaagtcgacaagaaagtcgagcctaagtcatgtgataaaacccat
acatgcccaagctgtcctgcaccagaatttctgggaggaccaagcgtgttcctgtttccacctaagcctaaagac
accctgatgattagtcgcactccagaggtgacctgtgtggtcgtggacgtcagccaggaggatcccgaagtgcag
tttaactggtacgtcgatggcgtggaagtccacaatgctaagacaaaaccaagagaggaacagttcaacagcaag
tacagggtcgtgtccgtgctgactgtcctgcatcaggactggctgaacggaaaagagtataagtgcaaagtgagc
aataagggcctgcccagttcaatcgagaaaacaatttccaaggcaaaaggccagcctcgggaaccacaggtgtat
accctgccacccagcagagaggaaatgacaaagaaccaggtgtcactgagctgtgccgtcaaaggcttttaccct
agcgatatcgctgtggagtgggaatccaatgggcagccagaaaacaattataagaccacacctccagtgctggac
agcgatggatccttctttctggtgtccaagctgaccgtggacaaatctcggtggcagcagggcaacgtgttctcc
tgctctgtcatgcatgaggccctgcacaatcattacacccagaaaagtctgtcactgagcaagtcctgtgataaa
acaggaggcgggggatctggcgggggaggcagtgggggaggcgggtccggaggaggaggaagcggaggaggagga
tccggaggaggcgggtctggaggcgggggaagtggaggaggaggatcttgcccaagttgtccagcacctgagttc
ctgggaggaccatccgtgttcctgtttccccctaagcccaaagacaccctgatgatcagccggacacctgaagtg
acttgcgtcgtggtggacgtgtcccaggaggatccagaggtccagtttaactggtatgtcgatggcgtggaggtc
cacaatgctaagaccaaacctcgcgaggaacagttcaactctgactaccgagtggtcagtgtgctgacagtcctg
catcaggattggctgaacgggaaggaatacaaatgtaaagtgagcaataagggactgccaagctccatcgagaag
accatcagcaaagccaaaggccagcccagggaacctcaggtgtacactctgccaccctcccgcgaggaaatgacc
aagaaccaggtgtctctgtggtgtctggtcaaaggattttatccctctgacatcgccgtggagtgggaaagtaat
ggccagcctgaaaacaattacaagactacccctccagtgctggactcagatgggagcttctttctgtatagtaag
ctgaccgtggataaatcacggtggcagcagggaaatgtgttctcttgcagtgtcatgcacgaggccctgcataac
cattacacacagaagtcattaagcttatcgggggaatgtggaggcggagggagcggcggaggcggcagcgatatc
gtgatgacacagtcacccctgtcgctccctgtgactcccggagagcctgcgtccatctcgtgccggtcaacaaag
tccctgctgcacagcaatggaattacgtatttgtactggtatttgcaaaaacccggacagagcccgcaattgttg
atctaccagatgtcaaaccttgcgagcggggtcccggatcgctttagctcgtcggggtcaggaactgacttcaca
ctcaaaatctcaagggtcgaggccgaggatgaaggtgtctattactgcgctcagaatctcgaaatcccgcggacc
tttggtggaggtacgaagctggagatcaagcgaacgggaggcggtgggtccggtggcggtggttccggcggaggt
ggttcgggaggaggaggtagccaggtgcagcttgtccagtcgggagcagaggtaaagaagccaggagagtcggtg
aaaatctcgtgtaaagcctcggggtacacattcacgaactacggcatgaattgggtgagacaagctcccgggcag
ggcttgaaatggatgggatggattaacacctacacaggggaatccacatatgcggacgacttcaaggggaggttc
gcgttttcacttgatacttcagcgagcacggcgtatctccaactctcgtcgcttcgctccgaagataccgcagta
tacttttgcgccagattcgcgattaaaggggactattggggacaggggacccttgtcacggtgtcatcatgataa
>Ab #6-HC-aa
(SEQ ID NO: 409)
QLQLQESGPGLVKPSETLSLTCTVSGGSISSSVYYWSWIRQPPGKGLEWIGVIYPSGNTYYSPSLKSRVTISVDT
SKNQFSLKLSSVTAADTAVYYCARTIYDLFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPSCPAPEFLGGPSVFLFPPKPKDTLMISRTPEVTCVVVDVSQEDPEVQFNWYVDGVEVHNAKTKPREEQFNSK
YRVVSVLTVLHQDWLNGKEYKCKVSNKGLPSSIEKTISKAKGQPREPQVYTLPPSREEMTKNQVSLSCAVKGFYP
SDIAVEWESNGQPENNYKTTPPVLDSDGSFFLVSKLTVDKSRWQQGNVFSCSVMHEALHNHYTQKSLSLSKSCDK
TGGGGSGGGGSGGGGSGGGGSGGGGSGGGGSGGGGSGGGGSCPSCPAPEFLGGPSVFLFPPKPKDTLMISRTPEV
TCVVVDVSQEDPEVQFNWYVDGVEVHNAKTKPREEQFNSDYRVVSVLTVLHQDWLNGKEYKCKVSNKGLPSSIEK
TISKAKGQPREPQVYTLPPSREEMTKNQVSLWCLVKGFYPSDIAVEWESNGQPENNYKTTPPVLDSDGSFFLYSK
LTVDKSRWQQGNVFSCSVMHEALHNHYTQKSLSLSGECGGGGSGGGGS
DIVMTQSPLSLPVTPGEPASISCRSTKSLLHSDGITYLYWYLQKPGQSPQLLIYQLSNLASGVPDRFSSSGSGTD
FTLKISRVEAEDEGVYYCAQNLEIPRTFGGGTKLEIKRTGGGGSGGGGSGGGGSGGGGSQVQLVQSGAEVKKPGE
SVKISCKASGYTFTNYGMNWVRQAPGQGLKWMGWINTYTGESTYADDFKGRFAFSLDTSASTAYLQLSSLRSEDT
AVYFCARFAIKGDYWGQGTLVTVSS**
>Ab #7-HC-aa
(SEQ ID NO: 410)
QLQLQESGPGLVKPSETLSLTCTVSGGSISSSVYYWSWIRQPPGKGLEWIGVIYPSGNTYYSPSLKSRVTISVDT
SKNQFSLKLSSVTAADTAVYYCARTIYDLFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPSCPAPEFLGGPSVFLFPPKPKDTLMISRTPEVTCVVVDVSQEDPEVQFNWYVDGVEVHNAKTKPREEQFNSK
YRVVSVLTVLHQDWLNGKEYKCKVSNKGLPSSIEKTISKAKGQPREPQVYTLPPSREEMTKNQVSLSCAVKGFYP
SDIAVEWESNGQPENNYKTTPPVLDSDGSFFLVSKLTVDKSRWQQGNVFSCSVMHEALHNHYTQKSLSLSKSCDK
TGGGGSGGGGSGGGGSGGGGSGGGGSGGGGSGGGGSGGGGSCPSCPAPEFLGGPSVFLFPPKPKDTLMISRTPEV
TCVVVDVSQEDPEVQFNWYVDGVEVHNAKTKPREEQFNSDYRVVSVLTVLHQDWLNGKEYKCKVSNKGLPSSIEK
TISKAKGQPREPQVYTLPPSREEMTKNQVSLWCLVKGFYPSDIAVEWESNGQPENNYKTTPPVLDSDGSFFLYSK
LTVDKSRWQQGNVFSCSVMHEALHNHYTQKSLSLSGECGGGGSGGGGSDIVMTQSPLSLPVTPGEPASISCRSTK
SLLHSDGITYLYWYLQKPGQSPQLLIYQLSN
LASGVPDRFSSSGSGTDFTLKISRVEAEDEGVYYCAQNLEIPRTFGCGTKLEIKRTGGGGSGGGGSGGGGS
GGGGSQVQLVQSGAEVKKPGESVKISCKASGYTFTNYGMNWVRQAPGQCLKWMGWINTYTGESTYADDFKG
RFAFSLDTSASTAYLQLSSLRSEDTAVYFCARFAIKGDYWGQGTLVTVSS**
>Ab #7-HC-nt
(SEQ ID NO: 411)
cagctgcagctgcaggagagcggacccggactggtcaagccctccgagacactgtctctgacctgcacagtgtca
ggcgggagcatcagctcctctgtctactattggtcttggattcggcagccccctggaaagggcctggaatggatc
ggcgtgatctacccaagtgggaacacttactattctcccagtctgaaatccagagtgaccatcagtgtcgacaca
tcaaagaatcagttcagcctgaaactgagttcagtgacagccgctgatactgcagtctactattgtgccaggacc
atctatgacctgtttgatatttgggggcagggaactatggtgaccgtcagctccgctagcactaagggaccttcc
gtgttcccactggccccctcaagcaaatctaccagtgggggaacagcagccctgggctgtctggtgaaggattac
tttccagagcccgtgaccgtcagttggaactcaggggctctgactagcggagtgcacaccttccccgcagtcctg
cagtcctctgggctgtactccctgagttcagtggtcactgtgcccagctcctctctgggaacacagacttatatc
tgcaacgtgaatcacaagccttccaataccaaagtcgacaagaaagtcgagcctaagtcatgtgataaaacccat
acatgcccaagctgtcctgcaccagaatttctgggaggaccaagcgtgttcctgtttccacctaagcctaaagac
accctgatgattagtcgcactccagaggtgacctgtgtggtcgtggacgtcagccaggaggatcccgaagtgcag
tttaactggtacgtcgatggcgtggaagtccacaatgctaagacaaaaccaagagaggaacagttcaacagcaag
tacagggtcgtgtccgtgctgactgtcctgcatcaggactggctgaacggaaaagagtataagtgcaaagtgagc
aataagggcctgcccagttcaatcgagaaaacaatttccaaggcaaaaggccagcctcgggaaccacaggtgtat
accctgccacccagcagagaggaaatgacaaagaaccaggtgtcactgagctgtgccgtcaaaggcttttaccct
agcgatatcgctgtggagtgggaatccaatgggcagccagaaaacaattataagaccacacctccagtgctggac
agcgatggatccttctttctggtgtccaagctgaccgtggacaaatctcggtggcagcagggcaacgtgttctcc
tgctctgtcatgcatgaggccctgcacaatcattacacccagaaaagtctgtcactgagcaagtcctgtgataaa
acaggaggcgggggatctggcgggggaggcagtgggggaggcgggtccggaggaggaggaagcggaggaggagga
tccggaggaggcgggtctggaggcgggggaagtggaggaggaggatcttgcccaagttgtccagcacctgagttc
ctgggaggaccatccgtgttcctgtttccccctaagcccaaagacaccctgatgatcagccggacacctgaagtg
acttgcgtcgtggtggacgtgtcccaggaggatccagaggtccagtttaactggtatgtcgatggcgtggaggtc
cacaatgctaagaccaaacctcgcgaggaacagttcaactctgactaccgagtggtcagtgtgctgacagtcctg
catcaggattggctgaacgggaaggaatacaaatgtaaagtgagcaataagggactgccaagctccatcgagaag
accatcagcaaagccaaaggccagcccagggaacctcaggtgtacactctgccaccctcccgcgaggaaatgacc
aagaaccaggtgtctctgtggtgtctggtcaaaggattttatccctctgacatcgccgtggagtgggaaagtaat
ggccagcctgaaaacaattacaagactacccctccagtgctggactcagatgggagcttctttctgtatagtaag
ctgaccgtggataaatcacggtggcagcagggaaatgtgttctcttgcagtgtcatgcacgaggccctgcataac
cattacacacagaagtcattaagcttatcgggggaatgtggaggcggagggagcggcggaggcggcagcgatatc
gtgatgacacagtcacccctgtcgctccctgtgactcccggagagcctgcgtccatctcgtgccggtcaacaaag
tccctgctgcacagcgacggaattacgtatttgtactggtatttgcaaaaacccggacagagcccgcaattgttg
atctaccagctctcaaaccttgcgagcggggtcccggatcgctttagctcgtcggggtcaggaactgacttcaca
ctcaaaatctcaagggtcgaggccgaggatgaaggtgtctattactgcgctcagaatctcgaaatcccgcggacc
tttggttgcggtacgaagctggagatcaagcgaacgggaggcggtgggtccggtggcggtggttccggcggaggt
ggttcgggaggaggaggtagccaggtgcagcttgtccagtcgggagcagaggtaaagaagccaggagagtcggtg
aaaatctcgtgtaaagcctcggggtacacattcacgaactacggcatgaattgggtgagacaagctcccgggcag
tgtttgaaatggatgggatggattaacacctacacaggggaatccacatatgcggacgacttcaaggggaggttc
gcgttttcacttgatacttcagcgagcacggcgtatctccaactgtcgtcgcttcgctccgaagataccgcagta
tacttttgcgccagattcgcgattaaaggggactattggggacaggggacccttgtcacggtgtcatcatgataa
>Ab #8-HC-aa
(SEQ ID NO: 412)
QLQLQESGPGLVKPSETLSLTCTVSGGSISSSVYYWSWIRQPPGKGLEWIGVIYPSGNTYYSPSLKSRVTISVDT
SKNQFSLKLSSVTAADTAVYYCARTIYDLFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPSCPAPEFLGGPSVFLFPPKPKDTLMISRTPEVTCVVVDVSQEDPEVQFNWYVDGVEVHNAKTKPREEQFNSK
YRVVSVLTVLHQDWLNGKEYKCKVSNKGLPSSIEKTISKAKGQPREPQVYTLPPSREEMTKNQVSLSCAVKGFYP
SDIAVEWESNGQPENNYKTTPPVLDSDGSFFLVSKLTVDKSRWQQGNVFSCSVMHEALHNHYTQKSLSLSKSCDK
TGGGGSGGGGSGGGGSGGGGSGGGGSGGGGSGGGGSGGGGSCPSCPAPEFLGGPSVFLFPPKPKDTLMISRTPEV
TCVVVDVSQEDPEVQFNWYVDGVEVHNAKTKPREEQFNSDYRVVSVLTVLHQDWLNGKEYKCKVSNKGLPSSIEK
TISKAKGQPREPQVYTLPPSREEMTKNQVSLWCLVKGFYPSDIAVEWESNGQPENNYKTTPPVLDSDGSFFLYSK
LTVDKSRWQQGNVFSCSVMHEALHNHYTQKSLSLSGECGGGGSGGGGSQVQLVQSGAEVKKPGESVKISCKASGY
TFTNYGMNWVRQAPGQGLKWMGWINTYTGESTYADDFKGRFAFSLDTSASTAYLQLSSLRSEDTAVYFCARFAIK
GDYWGQGTLVTVSSASTGGGGSGGGGSGGGGSGGGGSDIVMTQSPLSLPVTPGEPASISCRSTKSLLHSDGITYL
YWYLQKPGQSPQLLIYQMSNLASGVPDRFSSSGSGTDFTLKISRVEAEDEGVYYCAQNLEIPRTFGGGTKLEIKR
T**
>Ab #9-HC-aa
(SEQ ID NO: 413)
QLQLQESGPGLVKPSETLSLTCTVSGGSISSSVYYWSWIRQPPGKGLEWIGVIYPSGNTYYSPSLKSRVTISVDT
SKNQFSLKLSSVTAADTAVYYCARTIYDLFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPSCPAPEFLGGPSVFLFPPKPKDTLMISRTPEVTCVVVDVSQEDPEVQFNWYVDGVEVHNAKTKPREEQFNSK
YRVVSVLTVLHQDWLNGKEYKCKVSNKGLPSSIEKTISKAKGQPREPQVYTLPPSREEMTKNQVSLSCAVKGFYP
SDIAVEWESNGQPENNYKTTPPVLDSDGSFFLVSKLTVDKSRWQQGNVFSCSVMHEALHNHYTQKSLSLSKSCDK
TGGGGSGGGGSGGGGSGGGGSGGGGSGGGGSGGGGSGGGGSCPSCPAPEFLGGPSVFLFPPKPKDTLMISRTPEV
TCVVVDVSQEDPEVQFNWYVDGVEVHNAKTKPREEQFNSDYRVVSVLTVLHQDWLNGKEYKCKVSNKGLPSSIEK
TISKAKGQPREPQVYTLPPSREEMTKNQVSLWCLVKGFYPSDIAVEWESNGQPENNYKTTPPVLDSDGSFFLYSK
LTVDKSRWQQGNVFSCSVMHEALHNHYTQKSLSLSGECGGGGSGGGGSQVQLQQSGGGLVKPGESVKISCAASGY
TFTNYGMNWVKQAPGKGLKWMGWINTYTGESTYADDFKGRFTFSLETSASAAYLQINSLRPEDTAVYFCARFAIK
GDYWGQGTTVTVSSASTGGGGSGGGGSGGGGSGGGGSGDIVMTQSPSSLSASVGDKATITCRSTKSLLHSNGITY
LYWYLQKPGKAPKLLIYQMSNLA
SGVPDRFSSSGSGTEFTLTISSVQPEDEGTYYCAQNLEIPRTFGQGTKLEIK**
>Ab #10-HC-aa
(SEQ ID NO: 414)
QLQLQESGPGLVKPSETLSLTCTVSGGSISSSVYYWSWIRQPPGKGLEWIGVIYPSGNTYYSPSLKSRVTISVDT
SKNQFSLKLSSVTAADTAVYYCARTIYDLFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPSCPAPEFLGGPSVFLFPPKPKDTLMISRTPEVTCVVVDVSQEDPEVQFNWYVDGVEVHNAKTKPREEQFNSK
YRVVSVLTVLHQDWLNGKEYKCKVSNKGLPSSIEKTISKAKGQPREPQVYTLPPSREEMTKNQVSLSCAVKGFYP
SDIAVEWESNGQPENNYKTTPPVLDSDGSFFLVSKLTVDKSRWQQGNVFSCSVMHEALHNHYTQKSLSLSKSCDK
TGGGGSGGGGSGGGGSGGGGSGGGGSGGGGSGGGGSGGGGSCPSCPAPEFLGGPSVFLFPPKPKDTLMISRTPEV
TCVVVDVSQEDPEVQFNWYVDGVEVHNAKTKPREEQFNSDYRVVSVLTVLHQDWLNGKEYKCKVSNKGLPSSIEK
TISKAKGQPREPQVYTLPPSREEMTKNQVSLWCLVKGFYPSDIAVEWESNGQPENNYKTTPPVLDSDGSFFLYSK
LTVDKSRWQQGNVFSCSVMHEALHNHYTQKSLSLSGECGGGGSGGGGSQVQLQQSGGGLVKPGESVKISCAASGY
TFTNYGMNWVKQAPGKGLKWMGWINTYTGESTYADDFKGRFTFSLETSASAAYLQINSLRPEDTAVYFCARFAIK
GDYWGQGTTVTVSSASTGGGGSGGGGSGGGGSGGGGSGDIVMTQSPSSLSASVGDKATITCRSTKSLLHSDGITY
LYWYLQKPGKAPKLLIYQLSNLASGVPDRFSSSGSGTEFTLTISSVQPEDEGTYYCAQNLEIPRTFGQGTKLEIK
**
>Ab #11-HC-aa
(SEQ ID NO: 415)
QLQLQESGPGLVKPSETLSLTCTVSGGSISSSVYYWSWIRQPPGKGLEWIGVIYPSGNTYYSPSLKSRVTISVDT
SKNQFSLKLSSVTAADTAVYYCARTIYDLFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPSCPAPEFLGGPSVFLFPPKPKDTLMISRTPEVTCVVVDVSQEDPEVQFNWYVDGVEVHNAKTKPREEQFNSK
YRVVSVLTVLHQDWLNGKEYKCKVSNKGLPSSIEKTISKAKGQPREPQVYTLPPSREEMTKNQVSLSCAVKGFYP
SDIAVEWESNGQPENNYKTTPPVLDSDGSFFLVSKLTVDKSRWQQGNVFSCSVMHEALHNHYTQKSLSLSKSCDK
TGGGGSGGGGSGGGGSGGGGSGGGGSGGGGSGGGGSGGGGSCPSCPAPEFLGGPSVFLFPPKPKDTLMISRTPEV
TCVVVDVSQEDPEVQFNWYVDGVEVHNAKTKPREEQFNSDYRVVSVLTVLHQDWLNGKEYKCKVSNKGLPSSIEK
TISKAKGQPREPQVYTLPPSREEMTKNQVSLWCLVKGFYPSDIAVEWESNGQPENNYKTTPPVLDSDGSFFLYSK
LTVDKSRWQQGNVFSCSVMHEALHNHYTQKSLSLSGECGGGGSGGGGSDIVMTQSPSSLSASVGDKATITCRSTK
SLLHSNGITYLYWYLQKPGKAPKLLIYQMSNLASGVPDRFSSSGSGTEFTLTISSVQPEDEGTYYCAQNLEIPRT
FGQGTKLEIKRTGGGGSGGGGSGGGGSGGGGSGQVQLQQSGGGLVKPGESVKISCAASGYTFTNYGMNWVKQAPG
KGLKWMGWINTYTGESTYADDFKGRFTFSLETSASAAYLQINSLRPEDTAVYFCARFAIKGDYWGQGTTVTVSS*
*
>Ab #12-HC-aa
(SEQ ID NO: 416)
QLQLQESGPGLVKPSETLSLTCTVSGGSISSSVYYWSWIRQPPGKGLEWIGVIYPSGNTYYSPSLKSRVTISVDT
SKNQFSLKLSSVTAADTAVYYCARTIYDLFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPSCPAPEFLGGPSVFLFPPKPKDTLMISRTPEVTCVVVDVSQEDPEVQFNWYVDGVEVHNAKTKPREEQFNSK
YRVVSVLTVLHQDWLNGKEYKCKVSNKGLPSSIEKTISKAKGQPREPQVYTLPPSREEMTKNQVSLSCAVKGFYP
SDIAVEWESNGQPENNYKTTPPVLDSDGSFFLVSKLTVDKSRWQQGNVFSCSVMHEALHNHYTQKSLSLSKSCDK
TGGGGSGGGGSGGGGSGGGGSGGGGSGGGGSGGGGSGGGGSCPSCPAPEFLGGPSVFLFPPKPKDTLMISRTPEV
TCVVVDVSQEDPEVQFNWYVDGVEVHNAKTKPREEQFNSDYRVVSVLTVLHQDWLNGKEYKCKVSNKGLPSSIEK
TISKAKGQPREPQVYTLPPSREEMTKNQVSLWCLVKGFYPSDIAVEWESNGQPENNYKTTPPVLDSDGSFFLYSK
LTVDKSRWQQGNVFSCSVMHEALHNHYTQKSLSLSGECGGGGSGGGGSDIVMTQSPSSLSASVGDKATITCRSTK
SLLHSDGITYLYWYLQKPGKAPKLLIYQLSNLASGVPDRFSSSGSGTEFTLTISSVQPEDEGTYYCAQNLEIPRT
FGQGTKLEIKRTGGGGSGGGGSGGGGSGGGGSGQVQLQQSGGGLVKPGESVKISCAASGYTFTNYGMNWVKQAPG
KGLKWMGWINTYTGESTYADDFKGRFTFSLETSASAAYLQINSLRPEDTAVYFCARFAIKGDYWGQGTTVTVSS*
*
>Ab #13-HC-aa
(SEQ ID NO: 417)
QLQLQESGPGLVKPSETLSLTCTVSGGSISSSVYYWSWIRQPPGKGLEWIGVIYPSGNTYYSPSLKSRVTISVDT
SKNQFSLKLSSVTAADTAVYYCARTIYDLFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPSCPAPEFLGGPSVFLFPPKPKDTLMISRTPEVTCVVVDVSQEDPEVQFNWYVDGVEVHNAKTKPREEQFNSK
YRVVSVLTVLHQDWLNGKEYKCKVSNKGLPSSIEKTISKAKGQPREPQVYTLPPSREEMTKNQVSLSCAVKGFYP
SDIAVEWESNGQPENNYKTTPPVLDSDGSFFLVSKLTVDKSRWQQGNVFSCSVMHEALHNHYTQKSLSLSKSCDK
TGGGGSGGGGSGGGGSGGGGSGGGGSGGGGSGGGGSGGGGSCPSCPAPEFLGGPSVFLFPPKPKDTLMISRTPEV
TCVVVDVSQEDPEVQFNWYVDGVEVHNAKTKPREEQFNSDYRVVSVLTVLHQDWLNGKEYKCKVSNKGLPSSIEK
TISKAKGQPREPQVYTLPPSREEMTKNQVSLWCLVKGFYPSDIAVEWESNGQPENNYKTTPPVLDSDGSFFLYSK
LTVDKSRWQQGNVFSCSVMHEALHNHYTQKSLSLSGECGGGGSGGGGSDIVMTQSPSSLSASVGDKATITCRSTK
SLLHSNGITYLYWYLQKPGKAPKLLIYQMSNLASGVPDRFSSSGSGTEFTLTISSVQPEDEGTYYCAQNLEIPRT
FGQGTKLEIKRTPSHNSHQVPSAGGPTANSGTSGSQVQLQQSGGGLVKPGESVKISCAASGYTFTNYGMNWVKQA
PGKGLKWMGWINTYTGESTYADDFKGRFTFSLETSASAAYLQINSLRPEDTAVYFCARFAIKGDYWGQGTTVTVS
S**
>Ab #13-HC-nt
(SEQ ID NO: 418)
cagctgcagctgcaggagagcggacccggactggtcaagccctccgagacactgtctctgacctgcacagtgtca
ggcgggagcatcagctcctctgtctactattggtcttggattcggcagccccctggaaagggcctggaatggatc
ggcgtgatctacccaagtgggaacacttactattctcccagtctgaaatccagagtgaccatcagtgtcgacaca
tcaaagaatcagttcagcctgaaactgagttcagtgacagccgctgatactgcagtctactattgtgccaggacc
atctatgacctgtttgatatttgggggcagggaactatggtgaccgtcagctccgctagcactaagggaccttcc
gtgttcccactggccccctcaagcaaatctaccagtgggggaacagcagccctgggctgtctggtgaaggattac
tttccagagcccgtgaccgtcagttggaactcaggggctctgactagcggagtgcacaccttccccgcagtcctg
cagtcctctgggctgtactccctgagttcagtggtcactgtgcccagctcctctctgggaacacagacttatatc
tgcaacgtgaatcacaagccttccaataccaaagtcgacaagaaagtcgagcctaagtcatgtgataaaacccat
acatgcccaagctgtcctgcaccagaatttctgggaggaccaagcgtgttcctgtttccacctaagcctaaagac
accctgatgattagtcgcactccagaggtgacctgtgtggtcgtggacgtcagccaggaggatcccgaagtgcag
tttaactggtacgtcgatggcgtggaagtccacaatgctaagacaaaaccaagagaggaacagttcaacagcaag
tacagggtcgtgtccgtgctgactgtcctgcatcaggactggctgaacggaaaagagtataagtgcaaagtgagc
aataagggcctgcccagttcaatcgagaaaacaatttccaaggcaaaaggccagcctcgggaaccacaggtgtat
accctgccacccagcagagaggaaatgacaaagaaccaggtgtcactgagctgtgccgtcaaaggcttttaccct
agcgatatcgctgtggagtgggaatccaatgggcagccagaaaacaattataagaccacacctccagtgctggac
agcgatggatccttctttctggtgtccaagctgaccgtggacaaatctcggtggcagcagggcaacgtgttctcc
tgctctgtcatgcatgaggccctgcacaatcattacacccagaaaagtctgtcactgagcaagtcctgtgataaa
acaggaggcgggggatctggcgggggaggcagtgggggaggcgggtccggaggaggaggaagcggaggaggagga
tccggaggaggcgggtctggaggcgggggaagtggaggaggaggatcttgcccaagttgtccagcacctgagttc
ctgggaggaccatccgtgttcctgtttccccctaagcccaaagacaccctgatgatcagccggacacctgaagtg
acttgcgtcgtggtggacgtgtcccaggaggatccagaggtccagtttaactggtatgtcgatggcgtggaggtc
cacaatgctaagaccaaacctcgcgaggaacagttcaactctgactaccgagtggtcagtgtgctgacagtcctg
catcaggattggctgaacgggaaggaatacaaatgtaaagtgagcaataagggactgccaagctccatcgagaag
accatcagcaaagccaaaggccagcccagggaacctcaggtgtacactctgccaccctcccgcgaggaaatgacc
aagaaccaggtgtctctgtggtgtctggtcaaaggattttatccctctgacatcgccgtggagtgggaaagtaat
ggccagcctgaaaacaattacaagactacccctccagtgctggactcagatgggagcttctttctgtatagtaag
ctgaccgtggataaatcacggtggcagcagggaaatgtgttctcttgcagtgtcatgcacgaggccctgcataac
cattacacacagaagtcattaagcttatcgggagagtgcggtggcggagggagcggaggaggcgggtcggacatc
gtcatgacccagtcaccgtcctcactgtcggcgtcggtgggtgataaggccacaattacatgccgcagcacgaaa
tcactgctccactccaatgggattacatatctctattggtatctccaaaaacccggaaaggcacctaagttgctg
atctaccagatgtcgaacttggcatcgggagtacccgataggttctcgtcgtcgggaagcggcacggagttcacg
ctcaccatttcctcagtccagccggaggacgaaggaacttactactgcgctcagaatcttgaaatcccgcgcaca
tttggacaagggacgaaacttgaaatcaagcgaactccgtcccacaacagccatcaagtgccctcggcgggaggg
cccaccgccaattcggggacatcagggagccaggtacagttgcagcagtcgggaggcgggctggtaaaacctggt
gaaagcgtcaagatctcatgtgcagcctcagggtatacgttcaccaattacgggatgaactgggtgaagcaggcg
ccagggaaaggtcttaagtggatgggatggatcaacacttacacgggagagtccacatacgcggatgactttaag
gggcggttcacgttttcgttggagacttcagcgtccgctgcctacctccaaatcaactcccttagacccgaggac
acagcggtctatttctgtgcgcggtttgccattaaaggtgattattggggacagggtacgactgtgaccgtgtcc
agctgataa
cMet-his (bold is signal sequence)
(SEQ ID NO: 419)
MGAPAVLAPGILVILFTLVQRSNG
ECKEALAKSEMNVNMKYQLPNFTAETPIQNVILHEHHIFLGATNYIYVLNEEDLQKVAEYKTGPVLEHPDCFPCQ
DCSSKANLSGGVWKDNINMALVVDTYYDDQLISCGSVNRGTCQRHVFPHNHTADIQSEVHCIFSPQIEEPSQCPD
CVVSALGAKVLSSVKDRFINFFVGNTINSSYFPDHPLHSISVRRLKETKDGFMFLTDQSYIDVLPEFRDSYPIKY
VHAFESNNFIYFLTVQRETLDAQTFHTRIIRFCSINSGLHSYMEMPLECILTEKRKKRSTKKEVFNILQAAYVSK
PGAQLARQIGASLNDDILFGVFAQSKPDSAEPMDRSAMCAFPIKYVNDFFNKIVNKNNVRCLQHFYGPNHEHCFN
RTLLRNSSGCEARRDEYRTEFTTALQRVDLFMGQFSEVLLTSISTFIKGDLTIANLGTSEGRFMQVVVSRSGPST
PHVNFLLDSHPVSPEVIVEHTLNQNGYTLVITGKKITKIPLNGLGCRHFQSCSQCLSAPPFVQCGWCHDKCVRSE
ECLSGTWTQQICLPAIYKVFPNSAPLEGGTRLTICGWDFGFRRNNKFDLKKTRVLLGNESCTLTLSESTMNTLKC
TVGPAMNKHFNMSIIISNGHGTTQYSTFSYVDPVITSISPKYGPMAGGTLLTLTGNYLNSGNSRHISIGGKTCTL
KSVSNSILECYTPAQTISTEFAVKLKIDLANRETSIFSYREDPIVYEIHPTKSFISGGSTITGVGKNLNSVSVPR
MVINVHEAGRNFTVACQHRSNSEIICCTTPSLQQLNLQLPLKTKAFFMLDGILSKYFDLIYVHNPVFKPFEKPVM
ISMGNENVLEIKGNDIDPEAVKGEVLKVGNKSCENIHLHSEAVLCTVPNDLLKLNSELNIEWKQAISSTVLGKVI
VQPDQNFTGGGGSHHHHHH**
Human EpCAM-Fc-his tagged (bold is signal sequence)
(SEQ ID NO: 420)
MGWSLILLFLVAVATRVIS
QEECVCENYKLAVNCFVNNNRQCQCTSVGAQNTVICSKLAAKCLVMKAEMNGSKLGRRAKPEGALQNNDGLYDPD
CDESGLFKAKQCNGTSMCWCVNTAGVRRTDKDTEITCSERVRTYWIIIELKHKAREKPYDSKSLRTALQKEITTR
YQLDPKFITSILYENNVITIDLVQNSSQKTQNDVDIADVAYYFEKDVKGESLFHSKKMDLTVNGEQLDLDPGQTL
IYYVDEKAPEFSMQGLKIEGRMDPKSSDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGGGGGSHHHHHH**
Anti-c-Met-OA-5D5-HC-aa
(SEQ ID NO: 421)
EVQLVESGGGLVQPGGSLRLSCAASGYTFTSYWLHWVRQAPGKGLEWVGMIDPSNSDTRF
NPNFKDRFTISADTSKNTAYLQMNSLRAEDTAVYYCATYRSYVTPLDYWGQGTLVTVSS
Anti-c-Met-OA-5D5-LC-aa
(SEQ ID NO: 422)
DIQMTQSPSSLSASVGDRVTITCKSSQSLLYTSSQKNYLAWYQQKPGKAPKLLIYWASTR
ESGVPSRFSGSGSGTDFTLTISSLQPEDFATYYCQQYYAYPWTFGQGTKVEIKR
RESERVED: SEQ ID NO: 423, SEQ ID NO: 424
>Ab #X1-OA-5D5
(SEQ ID NO: 425)
EVQLVESGGGLVQPGGSLRLSCAASGYTFTSYWLHWVRQAPGKGLEWVGMIDPSNSDTRFNPNFKD
RFTISADTSKNTAYLQMNSLRAEDTAVYYCATYRSYVTPLDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKK
VEPKSCDKTHTCPSCPAPEFLGGPSVFLFPPKPKDTLMISRTPEVTCVVVDVSQEDPEVQFNWYVDGVEVHNAKT
KPREEQFNSKYRVVSVLTVLHQDWLNGKEYKCKVSNKGLPSSIEKTISKAKGQPREPQVYTLPPSREEMTKNQVS
LSCAVKGFYPSDIAVEWESNGQPENNYKTTPPVLDSDGSFFLVSKLTVDKSRWQQGNVFSCSVMHEALHNHYTQK
SLSLSKSCDKTGGGGSGGGGSGGGGSGGGGSGGGGSGGGGSGGGGSGGGGSCPSCPAPEFLGGPSVFLFPPKPKD
TLMISRTPEVTCVVVDVSQEDPEVQFNWYVDGVEVHNAKTKPREEQFNSDYRVVSVLTVLHQDWLNGKEYKCKVS
NKGLPSSIEKTISKAKGQPREPQVYTLPPSREEMTKNQVSLWCLVKGFYPSDIAVEWESNGQPENNYKTTPPVLD
SDGSFFLYSKLTVDKSRWQQGNVFSCSVMHEALHNHYTQKSLSLSGECGGGGSGGGGSDIVMTQSPLSLPVTPGE
PASISCRSTKSLLHSNGITYLYWYLQKPGQSPQLLIYQMSNLASGVPDRFSSSGSGTDFTLKISRVEAEDEGVYY
CAQNLEIPRTFGGGTKLEIKRTGGGGSGGGGSGGGGSGGGGSQVQLVQSGAEVKKPGESVKISCKASGYTFTNYG
MNWVRQAPGQGLKWMGWINTYTGESTYADDFKGRFAFSLDTSASTAYLQLSSLRSEDTAVYFCARFAIKGDYWGQ
GTLVTVSS**
>Ab #X2-OA-5D5
(SEQ ID NO: 426)
EVQLVESGGGLVQPGGSLRLSCAASGYTFTSYWLHWVRQAPGKGLEWVGMIDPSNSDTRFNPNFKD
RFTISADTSKNTAYLQMNSLRAEDTAVYYCATYRSYVTPLDYWGQGTLVTVSS
ASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPSCPAPEFLGGPSVFLFPPKPKDTLMISRTPEVTCVVVDVS
QEDPEVQFNWYVDGVEVHNAKTKPREEQFNSKYRVVSVLTVLHQDWLNGKEYKCKVSNKGLPSSIEKTISKAKGQ
PREPQVYTLPPSREEMTKNQVSLSCAVKGFYPSDIAVEWESNGQPENNYKTTPPVLDSDGSFFLVSKLTVDKSRW
QQGNVFSCSVMHEALHNHYTQKSLSLSKSCDKTGGGGSGGGGSGGGGSGGGGSGGGGSGGGGSGGGGSGGGGSCP
SCPAPEFLGGPSVFLFPPKPKDTLMISRTPEVTCVVVDVSQEDPEVQFNWYVDGVEVHNAKTKPREEQFNSDYRV
VSVLTVLHQDWLNGKEYKCKVSNKGLPSSIEKTISKAKGQPREPQVYTLPPSREEMTKNQVSLWCLVKGFYPSDI
AVEWESNGQPENNYKTTPPVLDSDGSFFLYSKLTVDKSRWQQGNVFSCSVMHEALHNHYTQKSLSLSGECGGGGS
GGGGSQVQLVQSGSELKKPGESVKVSCKASGYTFTNYGMHWVRQAPGQGLKWMGWINTYTGESTYADDFKGRFAF
SLDTSASTAYLQISSLKAEDTAVYFCARFAIKGDYWGQGTLVTVSSASTGGGGSGGGGSGGGGSGGGGSDIVMTQ
SPLSLPVTPGEPASISCRSTKSLLHSDGITYLYWYLQKPGQSPQLLIYQLSNLASGVPDRFSSSGSGTDFTLKIS
RVEAEDEGVYYCAQNLEIPYTFGQGTKVEIK**
>Ab #X3-OA-5D5
(SEQ ID NO: 427)
EVQLVESGGGLVQPGGSLRLSCAASGYTFTSYWLHWVRQAPGKGLEWVGMIDPSNSDTRFNPNFKD
RFTISADTSKNTAYLQMNSLRAEDTAVYYCATYRSYVTPLDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKK
VEPKSCDKTHTCPSCPAPEFLGGPSVFLFPPKPKDTLMISRTPEVTCVVVDVSQEDPEVQFNWYVDGVEVHNAKT
KPREEQFNSKYRVVSVLTVLHQDWLNGKEYKCKVSNKGLPSSIEKTISKAKGQPREPQVYTLPPSREEMTKNQVS
LSCAVKGFYPSDIAVEWESNGQPENNYKTTPPVLDSDGSFFLVSKLTVDKSRWQQGNVFSCSVMHEALHNHYTQK
SLSLSKSCDKTGGGGSGGGGSGGGGSGGGGSGGGGSGGGGSGGGGSGGGGSCPSCPAPEFLGGPSVFLFPPKPKD
TLMISRTPEVTCVVVDVSQEDPEVQFNWYVDGVEVHNAKTKPREEQFNSDYRVVSVLTVLHQDWLNGKEYKCKVS
NKGLPSSIEKTISKAKGQPREPQVYTLPPSREEMTKNQVSLWCLVKGFYPSDIAVEWESNGQPENNYKTTPPVLD
SDGSFFLYSKLTVDKSRWQQGNVFSCSVMHEALHNHYTQKSLSLSGECGGGGSGGGGSQVQLVQSGSELKKPGES
VKVSCKASGYTFTNYGMNWVRQAPGQGLKWMGWINTYTGESTYADDFKGRFAFSLDTSASTAYLQISSLKAEDTA
VYFCARFAIKGDYWGQGTLVTVSSASTGGGGSGGGGSGGGGSGGGGSDIVMTQSPLSLPVTPGEPASISCRSTKS
LLHSDGITYLYWYLQKPGQSPQLLIYQLSNLASGVPDRFSSSGSGTDFTLKISRVEAEDEGVYYCAQNLEIPRTF
GQGTKVEIK**
>Ab #X4-OA-5D5
(SEQ ID NO: 428)
EVQLVESGGGLVQPGGSLRLSCAASGYTFTSYWLHWVRQAPGKGLEWVGMIDPSNSDTRFNPNFKD
RFTISADTSKNTAYLQMNSLRAEDTAVYYCATYRSYVTPLDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKK
VEPKSCDKTHTCPSCPAPEFLGGPSVFLFPPKPKDTLMISRTPEVTCVVVDVSQEDPEVQFNWYVDGVEVHNAKT
KPREEQFNSKYRVVSVLTVLHQDWLNGKEYKCKVSNKGLPSSIEKTISKAKGQPREPQVYTLPPSREEMTKNQVS
LSCAVKGFYPSDIAVEWESNGQPENNYKTTPPVLDSDGSFFLVSKLTVDKSRWQQGNVFSCSVMHEALHNHYTQK
SLSLSKSCDKTGGGGSGGGGSGGGGSGGGGSGGGGSGGGGSGGGGSGGGGSCPSCPAPEFLGGPSVFLFPPKPKD
TLMISRTPEVTCVVVDVSQEDPEVQFNWYVDGVEVHNAKTKPREEQFNSDYRVVSVLTVLHQDWLNGKEYKCKVS
NKGLPSSIEKTISKAKGQPREPQVYTLPPSREEMTKNQVSLWCLVKGFYPSDIAVEWESNGQPENNYKTTPPVLD
SDGSFFLYSKLTVDKSRWQQGNVFSCSVMHEALHNHYTQKSLSLSGECGGGGSGGGGSDIVMTQSPLSLPVTPGE
PASISCRSTKSLLHSDGITYLYWYLQKPGQSPQLLIYQLSNLASGVPDRFSSSGSGTDFTLKISRVEAEDEGVYY
CAQNLEIPRTFGQGTKVEIKRTGGGGSGGGGSGGGGSGGGGSGQVQLVQSGSELKKPGESVKVSCKASGYTFTNY
GMNWVRQAPGQGLKWMGWINTYTGESTYADDFKGRFAFSLDTSASTAYLQISSLKAEDTAVYFCARFAIKGDYWG
QGTLVTVSS**
>Ab #X5-OA-5D5
(SEQ ID NO: 429)
EVQLVESGGGLVQPGGSLRLSCAASGYTFTSYWLHWVRQAPGKGLEWVGMIDPSNSDTRFNPNFKD
RFTISADTSKNTAYLQMNSLRAEDTAVYYCATYRSYVTPLDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKK
VEPKSCDKTHTCPSCPAPEFLGGPSVFLFPPKPKDTLMISRTPEVTCVVVDVSQEDPEVQFNWYVDGVEVHNAKT
KPREEQFNSKYRVVSVLTVLHQDWLNGKEYKCKVSNKGLPSSIEKTISKAKGQPREPQVYTLPPSREEMTKNQVS
LSCAVKGFYPSDIAVEWESNGQPENNYKTTPPVLDSDGSFFLVSKLTVDKSRWQQGNVFSCSVMHEALHNHYTQK
SLSLSKSCDKTGGGGSGGGGSGGGGSGGGGSGGGGSGGGGSGGGGSGGGGSCPSCPAPEFLGGPSVFLFPPKPKD
TLMISRTPEVTCVVVDVSQEDPEVQFNWYVDGVEVHNAKTKPREEQFNSDYRVVSVLTVLHQDWLNGKEYKCKVS
NKGLPSSIEKTISKAKGQPREPQVYTLPPSREEMTKNQVSLWCLVKGFYPSDIAVEWESNGQPENNYKTTPPVLD
SDGSFFLYSKLTVDKSRWQQGNVFSCSVMHEALHNHYTQKSLSLSGECGGGGSGGGGSDIVMTQSPLSLPVTPGE
PASISCRSTKSLLHSNGITYLYWYLQKPGQSPQLLIYQMSNLASGVPDRFSSSGSGTDFTLKISRVEAEDEGVYY
CAQNLEIPRTFGGGTKLEIKRTGGGGSGGGGSGGGGSGGGGSQVQLVQSGAEVKKPGESVKISCKASGYTFTNYG
MNWVRQAPGQGLKWMGWINTYTGESTYADDFKGRFAFSLDTSASTAYLQLSSLRSEDTAVYFCARFAIKGDYWGQ
GTLVTVSS**
>Ab #X6-OA-5D5
(SEQ ID NO: 430)
EVQLVESGGGLVQPGGSLRLSCAASGYTFTSYWLHWVRQAPGKGLEWVGMIDPSNSDTRFNPNFKD
RFTISADTSKNTAYLQMNSLRAEDTAVYYCATYRSYVTPLDYWGQGTLVTVSS
ASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPSCPAPEFLGGPSVFLFPPKPKDTLMISRTPEVTCVVVDVS
QEDPEVQFNWYVDGVEVHNAKTKPREEQFNSKYRVVSVLTVLHQDWLNGKEYKCKVSNKGLPSSIEKTISKAKGQ
PREPQVYTLPPSREEMTKNQVSLSCAVKGFYPSDIAVEWESNGQPENNYKTTPPVLDSDGSFFLVSKLTVDKSRW
QQGNVFSCSVMHEALHNHYTQKSLSLSKSCDKTGGGGSGGGGSGGGGSGGGGSGGGGSGGGGSGGGGSGGGGSCP
SCPAPEFLGGPSVFLFPPKPKDTLMISRTPEVTCVVVDVSQEDPEVQFNWYVDGVEVHNAKTKPREEQFNSDYRV
VSVLTVLHQDWLNGKEYKCKVSNKGLPSSIEKTISKAKGQPREPQVYTLPPSREEMTKNQVSLWCLVKGFYPSDI
AVEWESNGQPENNYKTTPPVLDSDGSFFLYSKLTVDKSRWQQGNVFSCSVMHEALHNHYTQKSLSLSGECGGGGS
GGGGSDIVMTQSPLSLPVTPGEPASISCRSTKSLLHSDGITYLYWYLQKPGQSPQLLIYQLSNLASGVPDRFSSS
GSGTDFTLKISRVEAEDEGVYYCAQNLEIPRTFGGGTKLEIKRTGGGGSGGGGSGGGGSGGGGSQVQLVQSGAEV
KKPGESVKISCKASGYTFTNYGMNWVRQAPGQGLKWMGWINTYTGESTYADDFKGRFAFSLDTSASTAYLQLSSL
RSEDTAVYFCARFAIKGDYWGQGTLVTVSS**
>Ab #X7-OA-5D5
(SEQ ID NO: 431)
EVQLVESGGGLVQPGGSLRLSCAASGYTFTSYWLHWVRQAPGKGLEWVGMIDPSNSDTRFNPNFKD
RFTISADTSKNTAYLQMNSLRAEDTAVYYCATYRSYVTPLDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKK
VEPKSCDKTHTCPSCPAPEFLGGPSVFLFPPKPKDTLMISRTPEVTCVVVDVSQEDPEVQFNWYVDGVEVHNAKT
KPREEQFNSKYRVVSVLTVLHQDWLNGKEYKCKVSNKGLPSSIEKTISKAKGQPREPQVYTLPPSREEMTKNQVS
LSCAVKGFYPSDIAVEWESNGQPENNYKTTPPVLDSDGSFFLVSKLTVDKSRWQQGNVFSCSVMHEALHNHYTQK
SLSLSKSCDKTGGGGSGGGGSGGGGSGGGGSGGGGSGGGGSGGGGSGGGGSCPSCPAPEFLGGPSVFLFPPKPKD
TLMISRTPEVTCVVVDVSQEDPEVQFNWYVDGVEVHNAKTKPREEQFNSDYRVVSVLTVLHQDWLNGKEYKCKVS
NKGLPSSIEKTISKAKGQPREPQVYTLPPSREEMTKNQVSLWCLVKGFYPSDIAVEWESNGQPENNYKTTPPVLD
SDGSFFLYSKLTVDKSRWQQGNVFSCSVMHEALHNHYTQKSLSLSGECGGGGSGGGGSDIVMTQSPLSLPVTPGE
PASISCRSTKSLLHSDGITYLYWYLQKPGQSPQLLIYQLSNLASGVPDRFSSSGSGTDFTLKISRVEAEDEGVYY
CAQNLEIPRTFGCGTKLEIKRTGGGGSGGGGSGGGGSGGGGSQVQLVQSGAEVKKPGESVKISCKASGYTFTNYG
MNWVRQAPGQCLKWMGWINTYTGESTYADDFKGRFAFSLDTSASTAYLQLSSLRSEDTAVYFCARFAIKGDYWGQ
GTLVTVSS**
>Ab #X8-OA-5D5
(SEQ ID NO: 432)
EVQLVESGGGLVQPGGSLRLSCAASGYTFTSYWLHWVRQAPGKGLEWVGMIDPSNSDTRFNPNFKD
RFTISADTSKNTAYLQMNSLRAEDTAVYYCATYRSYVTPLDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKK
VEPKSCDKTHTCPSCPAPEFLGGPSVFLFPPKPKDTLMISRTPEVTCVVVDVSQEDPEVQFNWYVDGVEVHNAKT
KPREEQFNSKYRVVSVLTVLHQDWLNGKEYKCKVSNKGLPSSIEKTISKAKGQPREPQVYTLPPSREEMTKNQVS
LSCAVKGFYPSDIAVEWESNGQPENNYKTTPPVLDSDGSFFLVSKLTVDKSRWQQGNVFSCSVMHEALHNHYTQK
SLSLSKSCDKTGGGGSGGGGSGGGGSGGGGSGGGGSGGGGSGGGGSGGGGSCPSCPAPEFLGGPSVFLFPPKPKD
TLMISRTPEVTCVVVDVSQEDPEVQFNWYVDGVEVHNAKTKPREEQFNSDYRVVSVLTVLHQDWLNGKEYKCKVS
NKGLPSSIEKTISKAKGQPREPQVYTLPPSREEMTKNQVSLWCLVKGFYPSDIAVEWESNGQPENNYKTTPPVLD
SDGSFFLYSKLTVDKSRWQQGNVFSCSVMHEALHNHYTQKSLSLSGECGGGGSGGGGSQVQLVQSGAEVKKPGES
VKISCKASGYTFTNYGMNWVRQAPGQGLKWMGWINTYTGESTYADDFKGRFAFSLDTSASTAYLQLSSLRSEDTA
VYFCARFAIKGDYWGQGTLVTVSSASTGGGGSGGGGSGGGGSGGGGSDIVMTQSPLSLPVTPGEPASISCRSTKS
LLHSDGITYLYWYLQKPGQSPQLLIYQMSNLASGVPDRFSSSGSGTDFTLKISRVEAEDEGVYYCAQNLEIPRTF
GGGTKLEIKRT**
>Ab #X9-OA-5D5
(SEQ ID NO: 433)
EVQLVESGGGLVQPGGSLRLSCAASGYTFTSYWLHWVRQAPGKGLEWVGMIDPSNSDTRFNPNFKD
RFTISADTSKNTAYLQMNSLRAEDTAVYYCATYRSYVTPLDYWGQGTLVTVSS
ASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPSCPAPEFLGGPSVFLFPPKPKDTLMISRTPEVTCVVVDVS
QEDPEVQFNWYVDGVEVHNAKTKPREEQFNSKYRVVSVLTVLHQDWLNGKEYKCKVSNKGLPSSIEKTISKAKGQ
PREPQVYTLPPSREEMTKNQVSLSCAVKGFYPSDIAVEWESNGQPENNYKTTPPVLDSDGSFFLVSKLTVDKSRW
QQGNVFSCSVMHEALHNHYTQKSLSLSKSCDKTGGGGSGGGGSGGGGSGGGGSGGGGSGGGGSGGGGSGGGGSCP
SCPAPEFLGGPSVFLFPPKPKDTLMISRTPEVTCVVVDVSQEDPEVQFNWYVDGVEVHNAKTKPREEQFNSDYRV
VSVLTVLHQDWLNGKEYKCKVSNKGLPSSIEKTISKAKGQPREPQVYTLPPSREEMTKNQVSLWCLVKGFYPSDI
AVEWESNGQPENNYKTTPPVLDSDGSFFLYSKLTVDKSRWQQGNVFSCSVMHEALHNHYTQKSLSLSGECGGGGS
GGGGSQVQLQQSGGGLVKPGESVKISCAASGYTFTNYGMNWVKQAPGKGLKWMGWINTYTGESTYADDFKGRFTF
SLETSASAAYLQINSLRPEDTAVYFCARFAIKGDYWGQGTTVTVSSASTGGGGSGGGGSGGGGSGGGGSGDIVMT
QSPSSLSASVGDKATITCRSTKSLLHSNGITYLYWYLQKPGKAPKLLIYQMSNLASGVPDRFSSSGSGTEFTLTI
SSVQPEDEGTYYCAQNLEIPRTFGQGTKLEIK**
>Ab #X10-OA-5D5
(SEQ ID NO: 434)
EVQLVESGGGLVQPGGSLRLSCAASGYTFTSYWLHWVRQAPGKGLEWVGMIDPSNSDTRFNPNFKDRFTISADTS
KNTAYLQMNSLRAEDTAVYYCATYRSYVTPLDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPSCPAPEFLGGPSVFLFPPKPKDTLMISRTPEVTCVVVDVSQEDPEVQFNWYVDGVEVHNAKTKPREEQFNS
KYRVVSVLTVLHQDWLNGKEYKCKVSNKGLPSSIEKTISKAKGQPREPQVYTLPPSREEMTKNQVSLSCAVKGFY
PSDIAVEWESNGQPENNYKTTPPVLDSDGSFFLVSKLTVDKSRWQQGNVFSCSVMHEALHNHYTQKSLSLSKSCD
KTGGGGSGGGGSGGGGSGGGGSGGGGSGGGGSGGGGSGGGGSCPSCPAPEFLGGPSVFLFPPKPKDTLMISRTPE
VTCVVVDVSQEDPEVQFNWYVDGVEVHNAKTKPREEQFNSDYRVVSVLTVLHQDWLNGKEYKCKVSNKGLPSSIE
KTISKAKGQPREPQVYTLPPSREEMTKNQVSLWCLVKGFYPSDIAVEWESNGQPENNYKTTPPVLDSDGSFFLYS
KLTVDKSRWQQGNVFSCSVMHEALHNHYTQKSLSLSGECGGGGSGGGGSQVQLQQSGGGLVKPGESVKISCAASG
YTFTNYGMNWVKQAPGKGLKWMGWINTYTGESTYADDFKGRFTFSLETSASAAYLQINSLRPEDTAVYFCARFAI
KGDYWGQGTTVTVSSASTGGGGSGGGGSGGGGSGGGGSGDIVMTQSPSSLSASVGDKATITCRSTKSLLHSDGIT
YLYWYLQKPGKAPKLLIYQLSNLASGVPDRFSSSGSGTEFTLTISSVQPEDEGTYYCAQNLEIPRTFGQGTKLEI
K**
>Ab #X11-OA-5D5
(SEQ ID NO: 435)
EVQLVESGGGLVQPGGSLRLSCAASGYTFTSYWLHWVRQAPGKGLEWVGMIDPSNSDTRFNPNFKD
RFTISADTSKNTAYLQMNSLRAEDTAVYYCATYRSYVTPLDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKK
VEPKSCDKTHTCPSCPAPEFLGGPSVFLFPPKPKDTLMISRTPEVTCVVVDVSQEDPEVQFNWYVDGVEVHNAKT
KPREEQFNSKYRVVSVLTVLHQDWLNGKEYKCKVSNKGLPSSIEKTISKAKGQPREPQVYTLPPSREEMTKNQVS
LSCAVKGFYPSDIAVEWESNGQPENNYKTTPPVLDSDGSFFLVSKLTVDKSRWQQGNVFSCSVMHEALHNHYTQK
SLSLSKSCDKTGGGGSGGGGSGGGGSGGGGSGGGGSGGGGSGGGGSGGGGSCPSCPAPEFLGGPSVFLFPPKPKD
TLMISRTPEVTCVVVDVSQEDPEVQFNWYVDGVEVHNAKTKPREEQFNSDYRVVSVLTVLHQDWLNGKEYKCKVS
NKGLPSSIEKTISKAKGQPREPQVYTLPPSREEMTKNQVSLWCLVKGFYPSDIAVEWESNGQPENNYKTTPPVLD
SDGSFFLYSKLTVDKSRWQQGNVFSCSVMHEALHNHYTQKSLSLSGECGGGGSGGGGSDIVMTQSPSSLSASVGD
KATITCRSTKSLLHSNGITYLYWYLQKPGKAPKLLIYQMSNLASGVPDRFSSSGSGTEFTLTISSVQPEDEGTYY
CAQNLEIPRTFGQGTKLEIKRTGGGGSGGGGSGGGGSGGGGSGQVQLQQSGGGLVKPGESVKISCAASGYTFTNY
GMNWVKQAPGKGLKWMGWINTYTGESTYADDFKGRFTFSLETSASAAYLQINSLRPEDTAVYFCARFAIKGDYWG
QGTTVTVSS**
>Ab #X12-OA-5D5
(SEQ ID NO: 436)
EVQLVESGGGLVQPGGSLRLSCAASGYTFTSYWLHWVRQAPGKGLEWVGMIDPSNSDTRFNPNFKD
RFTISADTSKNTAYLQMNSLRAEDTAVYYCATYRSYVTPLDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKK
VEPKSCDKTHTCPSCPAPEFLGGPSVFLFPPKPKDTLMISRTPEVTCVVVDVSQEDPEVQFNWYVDGVEVHNAKT
KPREEQFNSKYRVVSVLTVLHQDWLNGKEYKCKVSNKGLPSSIEKTISKAKGQPREPQVYTLPPSREEMTKNQVS
LSCAVKGFYPSDIAVEWESNGQPENNYKTTPPVLDSDGSFFLVSKLTVDKSRWQQGNVFSCSVMHEALHNHYTQK
SLSLSKSCDKTGGGGSGGGGSGGGGSGGGGSGGGGSGGGGSGGGGSGGGGSCPSCPAPEFLGGPSVFLFPPKPKD
TLMISRTPEVTCVVVDVSQEDPEVQFNWYVDGVEVHNAKTKPREEQFNSDYRVVSVLTVLHQDWLNGKEYKCKVS
NKGLPSSIEKTISKAKGQPREPQVYTLPPSREEMTKNQVSLWCLVKGFYPSDIAVEWESNGQPENNYKTTPPVLD
SDGSFFLYSKLTVDKSRWQQGNVFSCSVMHEALHNHYTQKSLSLSGECGGGGSGGGGSDIVMTQSPSSLSASVGD
KATITCRSTKSLLHSDGITYLYWYLQKPGKAPKLLIYQLSNLASGVPDRFSSSGSGTEFTLTISSVQPEDEGTYY
CAQNLEIPRTFGQGTKLEIKRTGGGGSGGGGSGGGGSGGGGSGQVQLQQSGGGLVKPGESVKISCAASGYTFTNY
GMNWVKQAPGKGLKWMGWINTYTGESTYADDFKGRFTFSLETSASAAYLQINSLRPEDTAVYFCARFAIKGDYWG
QGTTVTVSS**
>Ab #X13-OA-5D5
(SEQ ID NO: 437)
EVQLVESGGGLVQPGGSLRLSCAASGYTFTSYWLHWVRQAPGKGLEWVGMIDPSNSDTRFNPNFKD
RFTISADTSKNTAYLQMNSLRAEDTAVYYCATYRSYVTPLDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKK
VEPKSCDKTHTCPSCPAPEFLGGPSVFLFPPKPKDTLMISRTPEVTCVVVDVSQEDPEVQFNWYVDGVEVHNAKT
KPREEQFNSKYRVVSVLTVLHQDWLNGKEYKCKVSNKGLPSSIEKTISKAKGQPREPQVYTLPPSREEMTKNQVS
LSCAVKGFYPSDIAVEWESNGQPENNYKTTPPVLDSDGSFFLVSKLTVDKSRWQQGNVFSCSVMHEALHNHYTQK
SLSLSKSCDKTGGGGSGGGGSGGGGSGGGGSGGGGSGGGGSGGGGSGGGGSCPSCPAPEFLGGPSVFLFPPKPKD
TLMISRTPEVTCVVVDVSQEDPEVQFNWYVDGVEVHNAKTKPREEQFNSDYRVVSVLTVLHQDWLNGKEYKCKVS
NKGLPSSIEKTISKAKGQPREPQVYTLPPSREEMTKNQVSLWCLVKGFYPSDIAVEWESNGQPENNYKTTPPVLD
SDGSFFLYSKLTVDKSRWQQGNVFSCSVMHEALHNHYTQKSLSLSGECGGGGSGGGGSDIVMTQSPSSLSASVGD
KATITCRSTKSLLHSNGITYLYWYLQKPGKAPKLLIYQMSNLASGVPDRFSSSGSGTEFTLTISSVQPEDEGTYY
CAQNLEIPRTFGQGTKLEIKRTPSHNSHQVPSAGGPTANSGTSGSQVQLQQSGGGLVKPGESVKISCAASGYTFT
NYGMNWVKQAPGKGLKWMGWINTYTGESTYADDFKGRFTFSLETSASAAYLQINSLRPEDTAVYFCARFAIKGDY
WGQGTTVTVSS**
>Anti-EpCAM-HC1 aa
(SEQ ID NO: 438)
QVQLVQSGAEVKKPGASVKVSCKASGYTFTNYGMNWVRQAPGQGLKWMGWINTYTGESTYADDFKGRVTITADTS
ASTAYMELSSLRSEDTAVYYCARFAIKGDYWGQGTLVTVSS
>Anti-EpCAM-HC1 nt
(SEQ ID NO: 439)
caagtgcagttggtccagagcggtgcggaggtaaagaaacccggtgcatccgtgaaggtgtcgtgcaaagcctcc
gggtatacgttcacgaactatgggatgaactgggtcagacaagcaccgggtcagggactcaaatggatggggtgg
atcaatacatacacaggggaatcgacctacgcggatgactttaagggaagggtcaccattacggcggacacctcg
gcatcgactgcgtatatggaactctcatcacttcgctcggaggacacagccgtctactattgtgcgcggtttgcg
atcaagggagattactggggacagggaactttggtaacagtatcaagc
>Anti-EpCAM-HC2 aa
(SEQ ID NO: 440)
QVQLVQSGAEVKKPGASVKVSCKASGYTFTNYGMNWVRQAPGQGLKWMGWINTYTGESTYADDFKGRVTITLDTS
ASTAYMELSSLRSEDTAVYFCARFAIKGDYWGQGTLVTVSS
>Anti-EpCAM-HC2 nt
(SEQ ID NO: 441)
caagtgcagttggtccagagcggtgcggaggtaaagaaacccggtgcatccgtgaaggtgtcgtgcaaagcctcc
gggtatacgttcacgaactatgggatgaactgggtcagacaagcaccgggtcagggactcaaatggatggggtgg
atcaatacatacacaggggaatcgacctacgcggatgactttaagggaagggtcaccattacgttggacacctcg
gcatcgactgcgtatatggaactctcatcacttcgctcggaggacacagccgtctacttctgtgcgcggtttgcg
atcaagggagattactggggacagggaactttggtaacagtatcaagc
>anti-EpCAM-HC3 aa
(SEQ ID NO: 442)
QVQLQQSGGGLVKPGGSVKISCKASGYTFTNYGMNWVKQAPGKGLKWMGWINTYTGESTYADDFKGRFAFSLETS
ASAAYLQINSLRPEDTAVYFCARFAIKGDYWGQGTLVTVSS
>Anti-EpCAM-HC3 nt
(SEQ ID NO: 443)
caggtacagctgcagcaaagcgggggaggactcgtgaagcctggtggttcggtcaaaatctcgtgtaaagcgtca
gggtacaccttcacaaactatggtatgaactgggtgaaacaggcacccggaaagggtcttaagtggatgggctgg
atcaatacctacacgggggagtcgacatatgcggacgactttaaaggacggttcgcgttttcgttggagactagc
gcctccgctgcctacctccaaatcaatagccttaggccggaagatacggcggtctacttttgcgcaagatttgcc
attaagggggattattggggccaggggacgctggtgacagtcagctccgctagc
>anti-EpCAM-HC4 aa
(SEQ ID NO: 444)
QVQLVQSGAEVKKPGASVKVSCKASGYTFTNYGMNWVRQAPGQGLKWMGWINTYTGESTYADDFKGRFAFSLDTS
ASTAYMELSSLRSEDTAVYFCARFAIKGDYWGQGTLVTVSS
>anti-EpCAM-HC4 nt
(SEQ ID NO: 445)
caggtccagcttgtgcagtccggagccgaagtcaagaagccgggagccagcgtaaaggtgtcatgtaaggcgtcg
gggtatacattcacgaactacggtatgaattgggtgcgccaagctcccggacagggtttgaaatggatggggtgg
atcaacacgtatacaggggaatcaacttacgccgacgacttcaagggaaggttcgcattttcgttggatacatcg
gcgtccacggcgtacatggagctgtcaagcctgcggtcggaggacacggcggtatacttctgcgcaagatttgct
atcaaaggtgattattgggggcagggaaccctggtaaccgtgagcagc
>anti-EpCAM-HC5 aa
(SEQ ID NO: 446)
QVQLVQSGSELKKPGASVKVSCKASGYTFTNYGMNWVRQAPGQGLKWMGWINTYTGESTYADDFKGRFAFSLDTS
ASTAYLQISSLKAEDTAVYFCARFAIKGDYWGQGTLVTVSS
>anti-EpCAM-HC5 nt
(SEQ ID NO: 447)
caggtccagctggtgcagtcaggttcagagctgaagaagcccggagcgtccgtcaaagtgtcatgcaaggcctcg
ggttacacgtttacgaactacggtatgaattgggtccgccaggctccgggccagggactgaaatggatgggatgg
atcaacacatatactggtgaatccacgtatgcggacgactttaaggggagattcgcgttcagccttgatacatcg
gcgtcgaccgcgtacctccaaatctcgtccttgaaagcagaggacactgcagtatacttttgtgcccggttcgct
atcaagggagattattggggccaagggaccttggtgacagtgtccagc
>anti-EpCAM-HC6 aa
(SEQ ID NO: 448)
QVQLVQSGAEVKKPGASVKISCKASGYTFTNYGMNWVRQAPGQGLKWMGWINTYTGESTYADDFKGRFTITLDTS
ASTAYMELSSLRSEDTAVYYCARFAIKGDYWGQGTLVTVSS
>anti-EpCAM-HC6 nt
(SEQ ID NO: 449)
caagtccagttggtacaatcgggtgccgaggtaaagaaaccgggagcgtcggtcaaaatcagctgcaaggcctca
ggctatacctttacaaactacggcatgaattgggtgagacaggcacccgggcagggattgaaatggatgggttgg
atcaacacgtatacaggggagtccacctatgcagatgactttaaggggcgcttcactatcacgctcgacacgtcc
gcgtcgacggcgtacatggaactgtcatcgcttcggagcgaagatacagccgtgtactattgtgctaggtttgcg
attaagggagactactggggacagggaaccctcgtaactgtgtcatca
>anti-EpCAM-HC7 aa
(SEQ ID NO: 450)
QVQLVQSGAEVKKPGASVKISCKASGYTFTNYGMNWVRQAPGQGLKWMGWINTYTGESTYADDFKGRFAFSLDTS
ASTAYLQLSSLRSEDTAVYFCARFAIKGDYWGQGTLVTVSS
>anti-EpCAM-HC7 nt
(SEQ ID NO: 451)
caagtgcagctggtacaatcaggtgcggaggtgaaaaagccgggtgcctccgtcaagatttcatgcaaggcctcg
ggatacacattcaccaactatgggatgaattgggtgaggcaggctcccggtcaggggttgaaatggatggggtgg
attaacacatatacgggagaaagcacgtatgcggacgacttcaaggggcgcttcgcgttttccctggatacttcg
gcatcgactgcgtacctccagttgtcgtcgcttagatccgaggacacggccgtctacttctgtgcacggtttgca
atcaagggggattactggggacagggaacgcttgtaaccgtaagctca
>Anti-EpCAM-LC1 aa
(SEQ ID NO: 452)
DIVMTQSPLSLPVTPGEPASISCRSTKSLLHSNGITYLYWYLQKPGQSPQLLIYQMSNLASGVPDRFSGSGSGTD
FTLKISRVEAEDVGVYYCAQNLEIPRTFGQGTKVEIK
>Anti-EpCAM-LC1 nt
(SEQ ID NO: 453)
gacattgtcatgacgcagtcgccgctctcgctcccggtcacaccgggagaacccgcgtccatttcatgcagatcg
acaaagtcactcctccattcaaatggaatcacttacttgtactggtatcttcaaaaacccggtcagtcaccacag
ttgctcatctaccaaatgtccaatttggcttcgggagtgcccgaccgattcagcggttcggggagcggtacggat
tttacgttgaagatcagcagggtagaggcggaggacgtgggggtgtactattgtgcacagaaccttgaaattcca
cgcacctttggacaaggcaccaaggtcgaaatcaagcgtacggtggcggcaccttcagtgttcatctttccccct
tccgatgaacagctcaaaagcgggactgcatccgtagtctgtcttttgaacaacttctatcccagagaggcgaaa
gtacagtggaaggtggacaacgcccttcaatcaggcaatagccaggagtcggtgacggagcaggattccaaagat
agcacatactcgctttcatccactttgacattgtcgaaagcggactacgaaaagcacaaggtctatgcgtgcgag
gtgacgcaccagggactttcgtcgccggtaaccaagtcgttcaatcgcggggagtgctgataa
>Anti-EpCAM-LC2 aa
(SEQ ID NO: 454)
DIVMTQSPSSLSASVGDKATITCRSTKSLLHSNGITYLYWYLQKPGKAPKLLIYQMSNLASGVPDRFSSSGSGTD
FTLRISSVQPEDFATYYCAQNLEIPRTFGGGTKLEIK
>Anti-EpCAM-LC2 nt
(SEQ ID NO: 455)
gacattgtcatgacccagtcgccgtcatccctttcagcgagcgtgggggataaggcaacgatcacgtgtcgaagc
accaaatccttgctgcacagcaacgggattacgtatttgtactggtatttgcaaaaacccgggaaagccccgaag
ctcctcatctaccagatgtcgaatctggcgtcgggggtgccagatcggttctccagcagcgggtcgggtacagac
ttcacactccggatctcatcagtgcagcccgaggactttgcgacctactattgtgcccaaaatcttgaaatcccg
agaacgttcggaggtggcacgaaattggagattaaa
>anti-EpCAM-LC3 aa
(SEQ ID NO: 456)
DIVMTQSPLSLPVTPGEPASISCRSTKSLLHSNGITYLYWYLQKPGQSPQLLIYQMSNLASGVPDRFSSSGSGTD
FTLKISRVEAEDVGVYYCAQNLEIPRTFGQGTKVEIK
>anti-EpCAM-LC3 nt
(SEQ ID NO: 457)
gatatcgtaatgacccaatcgccgttgtcgcttccagtcacacccggggagcctgcttcgattagctgcagatca
acgaagtcgctcctccattcaaacgggattacgtatttgtactggtatcttcaaaagccgggtcagagcccgcag
ctgctgatctaccagatgtccaacttggcctcgggcgtccccgaccggtttagcagcagcgggtcgggaacggac
ttcactctcaagatctcaagggtcgaagcggaagatgtgggtgtgtattactgtgcgcagaatcttgagattccc
cgaacattcggtcagggaaccaaagtcgagatcaag
>anti-EpCAM-LC4 aa
(SEQ ID NO: 458)
DIVMTQSPLSLPVTPGEPASISCRSTKSLLHSNGITYLYWYLQKPGQSPQLLIYQMSNLASGVPDRFSSSGSGTD
FTLKISRVEAEDVGVYYCAQNLEIPRTFGGGTKLEIK
>anti-EpCAM-LC4 NT
(SEQ ID NO: 459)
Gacatcgtgatgacgcagtccccactgtcgctccctgtaacaccaggggagcccgcttccatttcgtgtaggtca
acgaagtccttgcttcatagcaatgggatcacttacttgtactggtatctccaaaaaccgggtcagtcccctcag
ttgctgatctaccagatgtcgaaccttgcgagcggtgtcccggatcgattttcatcgtccggatcgggaaccgac
ttcacactgaagattagccgcgtggaggccgaagatgtcggggtctactactgtgctcaaaaccttgagattccc
cggacgtttggaggcggcacgaagctggaaatcaag
>Anti-EpCAM scFv-v1 aa
(SEQ ID NO: 460)
QVQLVQSGSELKKPGESVKVSCKASGYTFTNYGMNWVRQAPGQCLKWMGWINTYTGESTYADDFKGRFAFSLDTS
ASTAYLQISSLKAEDTAVYFCARFAIKGDYWGQGTLVTVSSASTGGGGSGGGGSGGGGSGGGGSDIVMTQSPLSL
PVTPGEPASISCRSTKSLLHSDGITYLYWYLQKPGQSPQLLIYQLSNLASGVPDRFSSSGSGT
                                                          * *
DFTLKISRVEAEDEGVYYCAQNLEIPRTFGCGTKVEIK**
>Anti-EpCAM scFv-v1 nt
(SEQ ID NO: 461)
caggtacaactcgtgcagtcggggtcggagcttaagaagccgggagagtcggtaaaagtgtcgtgtaaagcctcc
gggtatacctttacgaattacggcatgaactgggtccggcaggcacccggtcagtgtctcaagtggatgggctgg
atcaatacgtacacgggcgaatccacttatgcagatgactttaagggaagattcgcgttctcattggacacatca
gcgtcaacggcgtatcttcaaatttcgtccttgaaagcggaggacacggcggtctatttctgcgcacgattcgct
atcaaaggagattactggggtcaggggacacttgtgacagtgtcgagcgccagcaccggaggtggagggagcgga
ggaggaggtagcggtggcggtggctcgggtggaggtggatcggacatcgtcatgactcagagccctctgtcactg
cccgtaactcccggggagcccgcctccattagctgtcggtcgacaaagtccttgctccactcagatggaatcacg
tatttgtactggtacttgcaaaagcctggacagtcgccacagctccttatctaccagttgtccaacctggcgtcg
ggagtaccggaccgcttttcatcatccgggtcaggcaccgatttcacactgaagattagccgcgtggaggcggag
gacgaaggggtctactactgcgcccagaacctcgaaattccgaggacctttggatgcggtactaaagtggaaatc
aagtgataa
>Anti-EpCAM scFv-v2 aa
(SEQ ID NO: 462)
QVQLVQSGSELKKPGESVKVSCKASGYTFTNYGMHWVRQAPGQGLKWMGWINTYTGESTYADDFKGRFAFSLDTS
ASTAYLQISSLKAEDTAVYFCARFAIKGDYWGQGTLVTVSSASTGGGGSGGGGSGGGGSGGGGSDIVMTQSPLSL
PVTPGEPASISCRSTKSLLHSDGITYLYWYLQKPGQSPQLLIYQLSNLASGVPDRFSSSGSGT
                                                          * *
DFTLKISRVEAEDEGVYYCAQNLEIPYTFGQGTKVEIK**
>Anti-EpCAM scFv-v2 nt
(SEQ ID NO: 463)
caggtacaactcgtgcagtcggggtcggagcttaagaagccgggagagtcggtaaaagtgtcgtgtaaagcctcc
gggtatacctttacgaattacggcatgcattgggtccggcaggcacccggtcaggggctcaagtggatgggctgg
atcaatacgtacacgggcgaatccacttatgcagatgactttaagggaagattcgcgttctcattggacacatca
gcgtcaacggcgtatcttcaaatttcgtccttgaaagcggaggacacggcggtctatttctgcgcacgattcgct
atcaaaggagattactggggtcaggggacacttgtgacagtgtcgagcgccagcaccggaggtggagggagcgga
ggaggaggtagcggtggcggtggctcgggtggaggtggatcggacatcgtcatgactcagagccctctgtcactg
cccgtaactcccggggagcccgcctccattagctgtcggtcgacaaagtccttgctccactcagatggaatcacg
tatttgtactggtacttgcaaaagcctggacagtcgccacagctccttatctaccagttgtccaacctggcgtcg
ggagtaccggaccgcttttcatcatccgggtcaggcaccgatttcacactgaagattagccgcgtggaggcggag
gacgaaggggtctactactgcgcccagaacctcgaaattccgtacacctttggacaaggtactaaagtggaaatc
aagtgataa
>Anti-EpCAM scFv-v3 aa
(SEQ ID NO: 464)
QVQLVQSGSELKKPGESVKVSCKASGYTFTNYGMNWVRQAPGQGLKWMGWINTYTGESTYADDFKGRFAFSLDTS
ASTAYLQISSLKAEDTAVYFCARFAIKGDYWGQGTLVTVSSASTGGGGSGGGGSGGGGSGGGGSDIVMTQSPLSL
PVTPGEPASISCRSTKSLLHSDGITYLYWYLQKPGQSPQLLIYQLSNLASGVPDRFSSSGSGT
                                                          * *
DFTLKISRVEAEDEGVYYCAQNLEIPRTFGQGTKVEIK **
>Anti-EpCAM scFv-v3 nt
(SEQ ID NO: 465)
caggtacaactcgtgcagtcggggtcggagcttaagaagccgggagagtcggtaaaagtgtcgtgtaaagcctcc
gggtatacctttacgaattacggcatgaactgggtccggcaggcacccggtcaggggctcaagtggatgggctgg
atcaatacgtacacgggcgaatccacttatgcagatgactttaagggaagattcgcgttctcattggacacatca
gcgtcaacggcgtatcttcaaatttcgtccttgaaagcggaggacacggcggtctatttctgcgcacgattcgct
atcaaaggagattactggggtcaggggacacttgtgacagtgtcgagcgccagcaccggaggtggagggagcgga
ggaggaggtagcggtggcggtggctcgggtggaggtggatcggacatcgtcatgactcagagccctctgtcactg
cccgtaactcccggggagcccgcctccattagctgtcggtcgacaaagtccttgctccactcagatggaatcacg
tatttgtactggtacttgcaaaagcctggacagtcgccacagctccttatctaccagttgtccaacctggcgtcg
ggagtaccggaccgcttttcatcatccgggtcaggcaccgatttcacactgaagattagccgcgtggaggcggag
gacgaaggggtctactactgcgcccagaacctcgaaattccgaggacctttggacaaggtactaaagtggaaatc
aagtgataa
>Anti-EpCAM scFv-v4 aa
(SEQ ID NO: 466)
DIVMTQSPLSLPVTPGEPASISCRSTKSLLHSDGITYLYWYLQKPGQSPQLLIYQLSNLASGVPDRFSSSG
                                                                * *
SGTDFTLKISRVEAEDEGVYYCAQNLEIPRTFGQGTKVEIKRTGGGGSGGGGSGGGGSGGGGSGQVQLVQSGSEL
KKPGESVKVSCKASGYTFTNYGMNWVRQAPGQGLKWMGWINTYTGESTYADDFKGRFAFSLDTSASTAYLQISSL
KAEDTAVYFCARFAIKGDYWGQGTLVTVSS**
>Anti-EpCAM scFv-v4 nt
(SEQ ID NO: 467)
gatatcgtgatgacacagtcacccctgtcgctccctgtgactcccggagagcctgcgtccatctcgtgccggtca
acaaagtccctgctgcacagcgacggaattacgtatttgtactggtatttgcaaaaacccggacagagcccgcaa
ttgttgatctaccagctctcaaaccttgcgagcggggtcccggatcgctttagctcgtcggggtcaggaactgac
ttcacactcaaaatctcaagggtcgaggccgaggatgaaggtgtctattactgcgctcagaatctcgaaatcccg
cggacctttggtcagggtacgaaggtagagatcaagcgaacgggaggcggtgggtccggtggcggtggttccggc
ggaggtggttcgggaggaggaggtagcggtcaggtgcagcttgtccagtcgggatcggagctcaagaagccagga
gagtcggtgaaagtatcgtgtaaagcctcggggtacacattcacgaactacggcatgaattgggtgagacaagct
cccgggcagggcttgaaatggatgggatggattaacacctacacaggggaatccacatatgcggacgacttcaag
gggaggttcgcgttttcacttgatacttcagcgagcacggcgtatctccaaatttcgtcgcttaaggcagaagat
accgcagtatacttttgcgccagattcgcgattaaaggggactattggggacaggggacccttgtcacggtgtca
tcatgataa
>Anti-EpCAM scFv-v5 aa
(SEQ ID NO: 468)
DIVMTQSPLSLPVTPGEPASISCRSTKSLLHSNGITYLYWYLQKPGQSPQLLIYQMSNLASGVPDRFSSSGSGTD
FTLKISRVEAEDEGVYYCAQNLEIPRTFGGGTKLEIKRTGGGGSGGGGSGGGGSGGGGSQVQLVQSGAEVKKPGE
SVKISCKASGYTFTNYGMNWVRQAPGQGLKWMGWINTYTGESTYADDFKGRFAFSLDTSASTAYLQLSSLRSEDT
AVYFCARFAIKGDYWGQGTLVTVSS**
>Anti-EpCAM scFv-v5 nt
(SEQ ID NO: 469)
gatatcgtgatgacacagtcacccctgtcgctccctgtgactcccggagagcctgcgtccatctcgtgccggtca
acaaagtccctgctgcacagcaatggaattacgtatttgtactggtatttgcaaaaacccggacagagcccgcaa
ttgttgatctaccagatgtcaaaccttgcgagcggggtcccggatcgctttagctcgtcggggtcaggaactgac
ttcacactcaaaatctcaagggtcgaggccgaggatgaaggtgtctattactgcgctcagaatctcgaaatcccg
cggacctttggtggaggtacgaagctggagatcaagcgaacgggaggcggtgggtccggtggcggtggttccggc
ggaggtggttcgggaggaggaggtagccaggtgcagcttgtccagtcgggagcagaggtaaagaagccaggagag
tcggtgaaaatctcgtgtaaagcctcggggtacacattcacgaactacggcatgaattgggtgagacaagctccc
gggcagggcttgaaatggatgggatggattaacacctacacaggggaatccacatatgcggacgacttcaagggg
aggttcgcgttttcacttgatacttcagcgagcacggcgtatctccaactctcgtcgcttcgctccgaagatacc
gcagtatacttttgcgccagattcgcgattaaaggggactattggggacaggggacccttgtcacggtgtcatca
tgataa
>Anti-EpCAM scFv-v6 aa
(SEQ ID NO: 470)
DIVMTQSPLSLPVTPGEPASISCRSTKSLLHSDGITYLYWYLQKPGQSPQLLIYQLSNLASGVPDRFSSSG
                                                                * *
SGTDFTLKISRVEAEDEGVYYCAQNLEIPRTFGGGTKLEIKRTGGGGSGGGGSGGGGSGGGGSQVQLVQSGAEVK
KPGESVKISCKASGYTFTNYGMNWVRQAPGQGLKWMGWINTYTGESTYADDFKGRFAFSLDTSASTAYLQLSSLR
SEDTAVYFCARFAIKGDYWGQGTLVTVSS**
>Anti-EpCAM scFv-v6 nt
(SEQ ID NO: 471)
gatatcgtgatgacacagtcacccctgtcgctccctgtgactcccggagagcctgcgtccatctcgtgccggtca
acaaagtccctgctgcacagcgacggaattacgtatttgtactggtatttgcaaaaacccggacagagcccgcaa
ttgttgatctaccagctctcaaaccttgcgagcggggtcccggatcgctttagctcgtcggggtcaggaactgac
ttcacactcaaaatctcaagggtcgaggccgaggatgaaggtgtctattactgcgctcagaatctcgaaatcccg
cggacctttggtggaggtacgaagctggagatcaagcgaacgggaggcggtgggtccggtggcggtggttccggc
ggaggtggttcgggaggaggaggtagccaggtgcagcttgtccagtcgggagcagaggtaaagaagccaggagag
tcggtgaaaatctcgtgtaaagcctcggggtacacattcacgaactacggcatgaattgggtgagacaagctccc
gggcagggcttgaaatggatgggatggattaacacctacacaggggaatccacatatgcggacgacttcaagggg
aggttcgcgttttcacttgatacttcagcgagcacggcgtatctccaactgtcgtcgcttcgctccgaagatacc
gcagtatacttttgcgccagattcgcgattaaaggggactattggggacaggggacccttgtcacggtgtcatca
tgataa
>Anti-EpCAM scFv-v7 aa
(SEQ ID NO: 472)
DIVMTQSPLSLPVTPGEPASISCRSTKSLLHSDGITYLYWYLQKPGQSPQLLIYQLSNLASGVPDRFSSSG
SGTDFTLKISRVEAEDEGVYYCAQNLEIPRTFGCGTKLEIKRTGGGGSGGGGSGGGGSGGGGSQVQLVQSGAEVK
KPGESVKISCKASGYTFTNYGMNWVRQAPGQCLKWMGWINTYTGESTYADDFKGRFAFSLDTSASTAYLQLSSLR
SEDTAVYFCARFAIKGDYWGQGTLVTVSS**
>Anti-EpCAM scFv-v7 nt
(SEQ ID NO: 473)
gatatcgtgatgacacagtcacccctgtcgctccctgtgactcccggagagcctgcgtccatctcgtgccggtca
acaaagtccctgctgcacagcgacggaattacgtatttgtactggtatttgcaaaaacccggacagagcccgcaa
ttgttgatctaccagctctcaaaccttgcgagcggggtcccggatcgctttagctcgtcggggtcaggaactgac
ttcacactcaaaatctcaagggtcgaggccgaggatgaaggtgtctattactgcgctcagaatctcgaaatcccg
cggacctttggttgcggtacgaagctggagatcaagcgaacgggaggcggtgggtccggtggcggtggttccggc
ggaggtggttcgggaggaggaggtagccaggtgcagcttgtccagtcgggagcagaggtaaagaagccaggagag
tcggtgaaaatctcgtgtaaagcctcggggtacacattcacgaactacggcatgaattgggtgagacaagctccc
gggcagtgtttgaaatggatgggatggattaacacctacacaggggaatccacatatgcggacgacttcaagggg
aggttcgcgttttcacttgatacttcagcgagcacggcgtatctccaactgtcgtcgcttcgctccgaagatacc
gcagtatacttttgcgccagattcgcgattaaaggggactattggggacaggggacccttgtcacggtgtcatca
tgataa
>Anti-EpCAM scFv-v8 aa
(SEQ ID NO: 474)
QVQLVQSGAEVKKPGESVKISCKASGYTFTNYGMNWVRQAPGQGLKWMGWINTYTGESTYADDFKGRFAFSLDTS
ASTAYLQLSSLRSEDTAVYFCARFAIKGDYWGQGTLVTVSSASTGGGGSGGGGSGGGGSGGGGSDIVMTQSPLSL
PVTPGEPASISCRSTKSLLHSDGITYLYWYLQKPGQSPQLLIYQMSNLASGVPDRFSSSGSGT
                                                          *
DFTLKISRVEAEDEGVYYCAQNLEIPRTFGGGTKLEIKRT**
>Anti-EpCAM scFv-v8 nt
(SEQ ID NO: 475)
caggtgcagctcgtccagtcaggggcggaggtcaagaaaccaggagagtcagtaaagatctcgtgcaaagcgtcg
ggatatacctttacaaactacggcatgaattgggtgcgacaagcacccggccagggcctgaagtggatggggtgg
atcaatacatatactggggagtccacttatgccgacgacttcaagggaaggtttgccttctccctggatacgtcg
gcgtcgaccgcttatttgcagttgagctcgctgaggtcggaagatacagcagtgtacttctgcgctcgcttcgca
atcaaaggggattactggggtcaggggacgcttgtaaccgtgtcctcagcgtcgacgggtggtggtgggtcggga
ggtggtggtagcggaggtggagggtcgggtggaggcggatcagatattgtgatgacacaatcgccgctctcactg
cccgtaacgcccggagagcccgcgtcaatttcatgtcggtcgacaaagtcactccttcactcggacgggattacg
tacctctattggtatcttcaaaagccgggtcagtcacctcaactcctcatctaccagatgtcgaacttggcatca
ggggtccctgatcgcttctcgtccagcgggtccgggacggactttaccttgaaaatctcaagagtggaggccgag
gacgaaggggtctactactgtgcccagaaccttgaaattccgcggacgtttggaggagggacaaagctggagatc
aaaagaacttgataa
>Anti-EpCAM scFv-v9 aa
(SEQ ID NO: 476)
QVQLQQSGGGLVKPGESVKISCAASGYTFTNYGMNWVKQAPGKGLKWMGWINTYTGESTYADDFKGRFTFSLETS
ASAAYLQINSLRPEDTAVYFCARFAIKGDYWGQGTTVTVSSASTGGGGSGGGGSGGGGSGGGGSGDIVMTQSPSS
LSASVGDKATITCRSTKSLLHSNGITYLYWYLQKPGKAPKLLIYQMSNLASGVPDRFSSSGSGTEFTLTISSVQP
EDEGTYYCAQNLEIPRTFGQGTKLEIK**
>Anti-EpCAM scFv-v9 nt
(SEQ ID NO: 477)
caggtacaactccagcaatcaggaggcgggcttgtaaaacccggggagtcggtgaagatcagctgtgcagcatca
gggtacacattcacaaactatggcatgaactgggtcaaacaggcgccagggaaggggctgaaatggatgggctgg
atcaatacgtacactggtgagagcacatacgcggatgatttcaaagggcggtttacgttttcactcgaaacgtcg
gcgtccgccgcatatcttcaaatcaattccttgaggccggaggacacagcagtctacttctgcgcccgatttgcc
attaagggtgattattggggccagggaaccacggtgactgtcagctccgcttcgacaggaggaggagggagcggt
ggaggaggatcgggaggtggagggtcgggtggtggtgggtcaggagacattgtgatgacccagtcgccctcgtcg
ctttcagcgagcgtcggggacaaggcgaccattacttgtcgctcaactaagtcgttgctgcactccaacgggatc
acgtacctctattggtatctccaaaaacctggtaaagcgcctaagctcctgatctaccagatgtccaatttggcg
tcgggagtaccggacagattttcaagctccggatcagggactgagttcacacttacgattagctccgtgcagccc
gaggatgaagggacctattactgcgcccagaacttggaaatcccgagaaccttcggacagggtacgaagctggaa
atcaagtgataa
>Anti-EpCAM scFv-v10 aa
(SEQ ID NO: 478)
QVQLQQSGGGLVKPGESVKISCAASGYTFTNYGMNWVKQAPGKGLKWMGWINTYTGESTYADDFKGRFTFSLETS
ASAAYLQINSLRPEDTAVYFCARFAIKGDYWGQGTTVTVSSASTGGGGSGGGGSGGGGSGGGGSGDIVMTQSPSS
LSASVGDKATITCRSTKSLLHSDGITYLYWYLQKPGKAPKLLIYQLSNLASGVPDRFSSSGSG
                                                            * *
TEFTLTISSVQPEDEGTYYCAQNLEIPRTFGQGTKLEIK**
>Anti-EpCAM scFv-v10 nt
(SEQ ID NO: 479)
caggtccagttgcaacagtccggaggtgggcttgtgaaacccggggagtcggtgaagatctcatgcgcggcttcg
gggtacacgttcacaaactacgggatgaattgggtcaagcaggcacccgggaaggggttgaaatggatgggttgg
atcaatacatacactggggagagcacgtatgcagatgacttcaaaggacgctttaccttcagcctcgaaacgagc
gcctcagccgcatacctccaaatcaatagcctccggccagaagatactgcggtgtatttctgcgctaggttcgcc
attaaaggggactattggggacaggggacgacggtgacggtatcatcagcgtcgactggtggaggcggctccgga
ggaggtgggtccggcggtggcgggtcaggtggtggtggctcgggagatattgtaatgacacagtccccgtcctcc
ctctccgcgagcgtgggagataaggccacgattacatgtcgaagcaccaaatcgctccttcactcggacggaatc
acatatttgtattggtacttgcaaaaaccggggaaggcgcctaagctgctgatctaccagctttcgaacctggcg
tcaggggtccctgaccggttttcgtcgtcgggaagcgggaccgagtttacacttacgatctcgtcagtacagccc
gaggacgaaggaacatactattgcgcccagaacttggagattccgagaacttttggccagggaaccaagctcgaa
atcaagtgataa
>Anti-EpCAM scFv-v11 aa
(SEQ ID NO: 480)
DIVMTQSPSSLSASVGDKATITCRSTKSLLHSNGITYLYWYLQKPGKAPKLLIYQMSNLASGVPDRFSSSGSGTE
FTLTISSVQPEDEGTYYCAQNLEIPRTFGQGTKLEIKRTGGGGSGGGGSGGGGSGGGGSGQVQLQQSGGGLVKPG
ESVKISCAASGYTFTNYGMNWVKQAPGKGLKWMGWINTYTGESTYADDFKGRFTFSLETSASAAYLQINSLRPED
TAVYFCARFAIKGDYWGQGTTVTVSS**
>Anti-EpCAM scFv-v11 nt
(SEQ ID NO: 481)
gacatcgtcatgacccagtcaccgtcctcactgtcggcgtcggtgggtgataaggccacaattacatgccgcagc
acgaaatcactgctccactccaatgggattacatatctctattggtatctccaaaaacccggaaaggcacctaag
ttgctgatctaccagatgtcgaacttggcatcgggagtacccgataggttctcgtcgtcgggaagcggcacggag
ttcacgctcaccatttcctcagtccagccggaggacgaaggaacttactactgcgctcagaatcttgaaatcccg
cgcacatttggacaagggacgaaacttgaaatcaagcgaactggaggaggtgggtcaggcggaggtgggagcggc
ggaggcggatcgggtggtggagggtcgggacaggtacagttgcagcagtcgggaggcgggctggtaaaacctggt
gaaagcgtcaagatctcatgtgcagcctcagggtatacgttcaccaattacgggatgaactgggtgaagcaggcg
ccagggaaaggtcttaagtggatgggatggatcaacacttacacgggagagtccacatacgcggatgactttaag
gggcggttcacgttttcgttggagacttcagcgtccgctgcctacctccaaatcaactcccttagacccgaggac
acagcggtctatttctgtgcgcggtttgccattaaaggtgattattggggacagggtacgactgtgaccgtgtcc
agctgataa
>Anti-EpCAM scFv-v12 aa
(SEQ ID NO: 482)
DIVMTQSPSSLSASVGDKATITCRSTKSLLHSDGITYLYWYLQKPGKAPKLLIYQLSNLASGVPDRFSSSG
SGTEFTLTISSVQPEDEGTYYCAQNLEIPRTFGQGTKLEIKRTGGGGSGGGGSGGGGSGGGGSGQVQLQQSGGGL
VKPGESVKISCAASGYTFTNYGMNWVKQAPGKGLKWMGWINTYTGESTYADDFKGRFTFSLETSASAAYLQINSL
RPEDTAVYFCARFAIKGDYWGQGTTVTVSS
>Anti-EpCAM scFv-v12 nt
(SEQ ID NO: 483)
gacatcgtcatgacccagtcaccgtcctcactgtcggcgtcggtgggtgataaggccacaattacatgccgcagc
acgaaatcactgctccactccgacgggattacatatctctattggtatctccaaaaacccggaaaggcacctaag
ttgctgatctaccagttgtcgaacttggcatcgggagtacccgataggttctcgtcgtcgggaagcggcacggag
ttcacgctcaccatttcctcagtccagccggaggacgaaggaacttactactgcgctcagaatcttgaaatcccg
cgcacatttggacaagggacgaaacttgaaatcaagcgaactggaggaggtgggtcaggcggaggtgggagcggc
ggaggcggatcgggtggtggagggtcgggacaggtacagttgcagcagtcgggaggcgggctggtaaaacctggt
gaaagcgtcaagatctcatgtgcagcctcagggtatacgttcaccaattacgggatgaactgggtgaagcaggcg
ccagggaaaggtcttaagtggatgggatggatcaacacttacacgggagagtccacatacgcggatgactttaag
gggcggttcacgttttcgttggagacttcagcgtccgctgcctacctccaaatcaactcccttagacccgaggac
acagcggtctatttctgtgcgcggtttgccattaaaggtgattattggggacagggtacgactgtgaccgtgtcc
agctgataa
>Anti-EpCAM scFv-v13 aa
(SEQ ID NO: 484)
DIVMTQSPSSLSASVGDKATITCRSTKSLLHSNGITYLYWYLQKPGKAPKLLIYQMSNLASGVPDRFSSSGSGTE
FTLTISSVQPEDEGTYYCAQNLEIPRTFGQGTKLEIKRTPSHNSHQVPSAGGPTANSGTSGSQVQLQQSGGGLVK
PGESVKISCAASGYTFTNYGMNWVKQAPGKGLKWMGWINTYTGESTYADDFKGRFTFSLETSASAAYLQINSLRP
EDTAVYFCARFAIKGDYWGQGTTVTVSS
>Anti-EpCAM scFv-v13 nt
(SEQ ID NO: 485)
gacatcgtcatgacccagtcaccgtcctcactgtcggcgtcggtgggtgataaggccacaattacatgccgcagc
acgaaatcactgctccactccaatgggattacatatctctattggtatctccaaaaacccggaaaggcacctaag
ttgctgatctaccagatgtcgaacttggcatcgggagtacccgataggttctcgtcgtcgggaagcggcacggag
ttcacgctcaccatttcctcagtccagccggaggacgaaggaacttactactgcgctcagaatcttgaaatcccg
cgcacatttggacaagggacgaaacttgaaatcaagcgaactccgtcccacaacagccatcaagtgccctcggcg
ggagggcccaccgccaattcggggacatcagggagccaggtacagttgcagcagtcgggaggcgggctggtaaaa
cctggtgaaagcgtcaagatctcatgtgcagcctcagggtatacgttcaccaattacgggatgaactgggtgaag
caggcgccagggaaaggtcttaagtggatgggatggatcaacacttacacgggagagtccacatacgcggatgac
tttaaggggcggttcacgttttcgttggagacttcagcgtccgctgcctacctccaaatcaactcccttagaccc
gaggacacagcggtctatttctgtgcgcggtttgccattaaaggtgattattggggacagggtacgactgtgacc
gtgtccagctgataa
>Murine anti-epCAM-LC
(SEQ ID NO: 486)
DIVMTQSAFS NPVTLGTSAS ISCRSTKSLL HSNGITYLYW YLQKPGQSPQ LLIYQMSNLA
SGVPDRFSSS GSGTDFTLRI SRVEAEDVGV YYCAQNLEIP RTFGGGTKLE IK
>Murine anti-epCAM-HC
(SEQ ID NO: 487)
QVQLQQSGPE LKKPGETVKI SCKASGYTFT NYGMNWVKQA PGRGLKWMGW INTYTGESTY
ADDFKGRFAF SLETSASAAY LQINNLKNED TATYFCARFA IKGDYWGQGT TLTVSS
>Humanized MOC31 from WO 2000/061635
(SEQ ID NO: 488)
DIQMTQSPSSLSASVGDRVTITCRSTKSLLHSNGITYLYWYQQKPGKAPKLLIYQMSNLASGVPSRFSSSGSGTD
FTLTISSLQPEDFATYYCAQNLEIPRTFGQGTKVELKRTPSHNSHQVPSAGGPTANSGTSGSEVQLVQSGPGLVQ
PGGSVRISCAASGYTFTNYGMNWVKQAPGKGLEWMGWINTYTGESTYADSFKGRFTFSLDTSASAAYLQINSLRA
EDTAVYYCARFAIKGDYWGQGTLLTVSS
>TFcBA large chain complete
(SEQ ID NO: 489)
QLQLQESGPGLVKPSETLSLTCTVSGGSISSSVYYWSWIRQPPGKGLEWIGVIYPSGNTYYSPSLKSRVTISVDT
SKNQFSLKLSSVTAADTAVYYCARTIYDLFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPSCPAPEFLGGPSVFLFPPKPKDTLMISRTPEVTCVVVDVSQEDPEVQFNWYVDGVEVHNAKTKPREEQFNSK
YRVVSVLTVLHQDWLNGKEYKCKVSNKGLPSSIEKTISKAKGQPREPQVYTLPPSREEMTKNQVSLSCAVKGFYP
SDIAVEWESNGQPENNYKTTPPVLDSDGSFFLVSKLTVDKSRWQQGNVFSCSVMHEALHNHYTQKSLSLSKSCDK
TGGGGSGGGGSGGGGSGGGGSGGGGSGGGGSGGGGSGGGGSCPSCPAPEFLGGPSVFLFPPKPKDTLMISRTPEV
TCVVVDVSQEDPEVQFNWYVDGVEVHNAKTKPREEQFNSDYRVVSVLTVLHQDWLNGKEYKCKVSNKGLPSSIEK
TISKAKGQPREPQVYTLPPSREEMTKNQVSLWCLVKGFYPSDIAVEWESNGQPENNYKTTPPVLDSDGSFFLYSK
LTVDKSRWQQGNVFSCSVMHEALHNHYTQKSLSLSGECGGGGSGGGGSDIVMTQSPLSLPVTPGEPASISCRSTK
SLLHSDGITYLYWYLQKPGQSPQLLIYQLSNLASGVPDRFSSSGSGTDFTLKISRVEAEDEGVYYCAQNLEIPRT
FGCGTKLEIKRTGGGGSGGGGSGGGGSDIQMTQSPSSLSASVGDRVTITCRSTKSLLHSNGITYLYWYQQKPGKA
PKLLIYQMSNLASGVPSRFSSSGSGTDFTLTISSLQPEDFATYYCAQNLEIPRTFGQGTKVELKRTPSHNSHQVP
SAGGPTANSGTSGSEVQLVQSGPGLVQPGGSVRISCAASGYTFTNYGMNWVKQAPGKGLEWMGWINTYTGESTYA
DSFKGRFTFSLDTSASAAYLQINSLRAEDTAVYYCARFAIKGDYWGQGTLLTVSS
```

### Patient Populations

Provided herein are effective methods for treating cancer in a patient, such as a human patient, and for selecting patients to be so treated. In one embodiment, the patient, such as a human patient, suffers from a cancer selected from the group consisting of non-small cell lung cancer (NSCLC), renal cell carcinoma (RCC), melanoma (e.g., cutaneous or intraocular malignant melanoma), colorectal cancer, serous ovarian carcinoma, liver cancer, bone cancer, pancreatic cancer, skin cancer, cancer of the head or neck, breast cancer, lung cancer, uterine cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), spinal axis tumor, glioma, pituitary adenoma, Kaposi's sarcoma, epidermoid cancer, squamous cell cancer, and mesothelioma. The disclosed methods are also applicable to treatment of metastatic cancers.

In one embodiment, a patient, such as a human patient, to be selected for treatment and/or treated in accordance with the disclosed methods has evidence of recurrent or persistent disease following primary chemotherapy.

The following examples are merely illustrative and should not be construed as limiting the scope of this disclosure in any way as many variations and equivalents will become apparent to those skilled in the art upon reading the present disclosure.

### EXAMPLES

Throughout the examples, the following materials and methods are used unless otherwise stated. In general, the practice of the techniques of the present disclosure employs, unless otherwise indicated, conventional techniques of medicine, chemistry, molecular biology, recombinant DNA technology, immunology (especially, e.g., antibody technology), pharmacology, pharmacy, and standard techniques of polypeptide preparation.

### Materials and Methods

The following cell lines are obtained from American Type Culture Collection (ATCC); Manassas, Va., to generate FFPE preparations of cell lines: NCI-H441 (or H441) (ATCC® HTB-174™), A549 (ATCC® CCL-185™), TOV-112D (ATCC® CRL-11731™) NCI-H1703, (or H1703) (ATCC® CRL-5889™), NCI-H2023 (or H2023) (ATCC® CRL-5912™), RKO (ATCC® CRL-2577™), A-204 (or A204) (ATCC® HTB-82™), U-87 MG (ATCC® HTB-14™), SK-OV-3 (or SKOV-3; SKOV3) (ATCC® HTB-77™), NCI-H1993 (or H1993) (ATCC® CRL-5909™), and ACHN (ATCC® CRL-1611™).

Cells are cultured to mid-log phase and then harvested using Trypsin-EDTA (0.05%), phenol red (Gibco® Life Technologies, Grand Island, N.Y.; Catalog #25300054). The trypsin is neutralized with serum-containing medium and the cells are centrifuged and washed with phosphate buffered saline (PBS), pH 7.4, twice. Cells are fixed using neutral buffered formalin (NBF) for 24 hours at room temperature. The cells are then washed with PBS twice prior to preparing a 1:1 mixture of PBS and warm Histogel® (Richard-Allan Scientific™, San Diego, Calif., Cat# HG-4000-0122) such that the estimated density is no less than 10 million cells per ml of the mixture. The mixture of cells in PBS and Histogel is quickly resuspended then placed in a container and allowed to solidify prior to transfer to 70% ethanol and processing for paraffin embedding. Once the cells have been embedded, a core is removed from embedded block and standard tissue microarray generation techniques are employed, to produce a cell microarray for pathologist training (regarding appropriate scoring for the target).

### Detection of Biomarkers for Bispecific Anti-c-Met, Anti-EpCAM Antibodies in FFPE Tissues

The following protocols are suitable for the detection of protein and/or mRNA in properly stored formalin fixed paraffin embedded tissues. Scoring of tissue based biomarkers is performed by a trained pathologist familiar with staining patterns observed with IHC and RNA ISH. Alternatively, image analysis methods such as those described in co-pending Application No. WO2013192457 (“Marker quantitation in single cells in tissue sections”) may be used. For each approach, the cut-point of each assay to define positivity is achieved by scoring ≧10%, ≧25%, ≧50%, ≧75%, or ≧90% of tumor cells staining weakly, moderately, or strongly based on the best differentiation of patient outcomes when treated with Ab#7 or any other MET inhibitor.

### Example 1: Quantitative Flow Cytometry Assessment of Cell Surface Expression of Antigens Targeted by Bispecific Antibodies

For measurements of cell surface expression of c-Met and EpCAM levels, quantitative flow cytometry is performed using the Quantum™ Simply Cellular® kit (Bangs Laboratories).

Cells are grown in exponential phase using standard cell culture media containing 10% FBS, and are passaged at least twice before the start of the experiment. On the day of the experiment, cells are visually assessed under a microscope to confirm between 60% and 80% confluence. Cells are detached from the culture plate by addition of 0.05% trypsin-EDTA (Gibco®), and once a majority of cells are detached (as assessed visually by microscope) the trypsin is inactivated using cell culture medium containing 10% FBS. The cells are centrifuged at about 500 g, resuspended in flow cytometry buffer (2% FBS+0.1% sodium azide in PBS), and seeded at a density of about 50,000 cells per well in a 96-well plate (BD Biosciences, catalog #62406-015).

In a separate 96-well plate, 2 drops of Quantum™ Simply Cellular® anti-mouse IgG coated beads (Bangs Laboratories, catalog #815) or anti-human IgG coated beads (Bangs Laboratories, catalog #816) are added per well. Each bead kit contains 5 bead populations (1 blank and 4 beads with increasing levels of Fc-specific capture antibody). Each coated population binds a specific number of monoclonal antibodies of the appropriate species (the “ABC” value), and thus serves as a standard curve for quantification when beads are labeled to saturation with the same monoclonal antibody that is used to label cell surface protein.

Antibodies against the cell surface targets are given in Table 1. An antibody against c-Met is conjugated with Alexa Fluor® 647 (Life Technologies, catalog #A-20006) according to manufacturer's instructions. Antibodies against EpCAM and CD44 are available pre-conjugated with fluorophores.

TABLE 1
Quantitative flow cytometry assessment of cell
surface expression for EpCAM and c-Met.

| Target | Exemplary Antibody | Reference |
|---|---|---|
| c-Met | 224G11-TH7-Hz3 | U.S. Patent Pub. No. 2011/0097262: |
| | | SEQ ID NO: 4 (V_{H} domain), |
| | | 10 (V_{L} domain), and 28 (hinge region) |
| EpCAM | EpCAM APC clone | BD Biosciences, catalog #347200 |
| | EBA-1 | |

The appropriate fluorophore-conjugated antibody is added to the cells and to the beads (200 nM antibody concentration in 80 μl of flow cytometry buffer) and is incubated for 30 minutes at 4° C. The plates are centrifuged and washed twice with 100 μl of ice-cold flow cytometry buffer (2% fetal bovine serum+0.1% sodium azide in PBS, pH 7.4). After the last wash, the cells and beads are centrifuged and resuspended in 1004 μl of ice-cold flow cytometry buffer and read using the appropriate fluorescence filter on a flow cytometer (BD FACSCanto™). Channel values for the bead populations are recorded in the bead lot-specific QuickCal® template provided in the Quantum™ Simply Cellular® kit. A regression is performed that relates fluorescence signal to the beads' ABC values. ABC values are assigned to stained cell samples using this standard curve. If monovalent antibody-to-cell surface receptor binding is presumed, then the ABC value equals the number of surface receptors.

Table 2 lists cell surface expression levels in a panel of cell lines derived from colorectal, ovarian, lung, breast, brain, gastric, prostate, and pancreatic cancer cell lines measured using the above protocol. Also listed is the ratio of expression between EpCAM and c-Met.

In the following Table, the Tumor Types are indicated as follows: 1=renal, 2=ovarian, 3=non-small cell lung Cell lines indicated are commercially available, e.g., from ATCC.

TABLE 2
Exemplary Cell Lines

| | | c-Met | EpCAM | |
|---|---|---|---|---|
| Cell Line | Tumor Type | (#/cell) | (#/cell) | EpCAM/c-Met |
| ACHN | 1 | 1.30E+05 | 4.60E+05 | 3.54 |
| SkOV3 | 2 | 1.60E+05 | 3.20E+05 | 2.00 |
| TOV112D | 2 | 2.72E+04 | 2.42E+03 | 0.09 |
| A549 | 3 | 7.20E+04 | 2.40E+04 | 0.33 |
| H1703 | 3 | 3.40E+04 | 1.20E+03 | 0.04 |
| H1993 | 3 | 6.10E+05 | 2.60E+06 | 4.26 |
| H441 | 3 | 3.20E+05 | 2.50E+06 | 7.81 |

As shown in the Table, EpCAM has the highest median expression level of any measured target, and also the highest median expression ratio relative to c-Met, supporting the selection of EpCAM as a targeting moiety for potent bispecific antibody binding to tumor cells expressing c-Met.

### Example 2: Xenograft Experiments

To explore the role of tumor c-Met, HGF, and EpCAM expression in promoting the in vivo activity of Antibody #7 (Ab#7), the molecule is evaluated in c-Met driven tumor models. For HGF-ligand dependent tumor models in mice, cell lines require autocrine secretion of human HGF, as it is known that mouse HGF does not activate human c-Met.

One such autocrine HGF model, U-87, was evaluated for in vivo activity of AB #7. Quantitative flow cytometry measurements of c-Met and EpCAM demonstrate that this model has expression corresponding to a 1+ and 0 IHC for c-Met and EpCAM, respectively, as shown in Table 2 and FIGS. 3 (HGF RNA ISH), 5 (c-Met IHC) and 7 (EpCAM IHC).

HCC827 cells transfected with human HGF (HCC827-HGF cells), along with mock-transfected HCC827 cells, were obtained from Dr. Jeffrey A. Engelman and were created according to the protocol described in Okamoto et al., Mol. Cancer Ther. 9(10):2785-92. Quantitative flow cytometry measurements of HCC827 cell line variants demonstrated that the level of c-Met decreased in the HGF-transfected cells relative to mock-transfected parental cells (1.2×10⁵/cell versus 3.8×10⁵/cell), but that the level of EpCAM was unaffected (2.2×10⁶/cell versus 2.2×10⁶/cell).

In addition, H441, a ligand-independent cell line where c-Met signaling is active in the absence of HGF, was selected to evaluate the in vivo activity of Ab#7 when HGF is not present, but c-Met is expressed at high levels. Quantitative flow cytometry measurements of c-Met and EpCAM demonstrate that this model has expression of corresponding to a 3+ and 3+ IHC for c-Met and EpCAM as shown in Table 2 and FIG. 5 (HGF) and 7 (EpCAM).

For in vivo studies, cells are cultured in T75 flasks under a humidified atmosphere of 5% CO₂ at 37° C. in RPMI 1640 medium supplemented with 10% fetal bovine serum (FBS). Cells are harvested by exposure to 0.25% trypsin. Cells are washed twice in phosphate-buffered saline (PBS) and resuspended in a 1:1 mixture of PBS and growth factor reduced Matrigel® (BD Biosciences) at. Viability is assessed with Trypan Blue (Life Technologies, catalog #15250-061) and mice are not implanted if the viability is <90%.

The in vivo activity of Ab#7, and control antibody OA-5D5 (humanized, anti-human-c-Met monoclonal antibody OA-5D5 is disclosed in U.S. Pat. No. 8,361,744 as heavy chain SEQ ID NO:45 and light chain SEQ ID NO:46) in these tumor models is evaluated essentially as follows: Six-to-seven-week-old female Nu/Nu mice (Charles River Laboratories, Wilmington, Mass.) are injected subcutaneously with 5×10⁶ HCC827-HGF, 5×10⁶ H441, or 5×10⁶ U-87 MG cells/mouse using an injection volume of 200 μl PBS. At seven days post-injection, initial tumor volumes are measured in two directions with fine calipers and volume calculated using the following formula: (π/6)*L*W². When the initial tumor volume reaches a range of 200±50 mm³, the animals are sorted into treatment groups of eight animals per group with tumor volumes randomized in each group. Mice are treated with PBS control, bispecific antibody, or OA-5D5 by intraperitoneal injection every 7 days.

The dose of OA-5D5 is approximately 10 mg/kg (an equal molar level with the 12 mg/kg bispecific antibody dose). Tumor volumes and body weights are determined twice weekly throughout the study. Tumor size data are plotted to represent mean and standard error of the mean for each measurement. Upon study completion, mice are euthanized and tumors from all animals are excised, flash frozen in liquid N₂, and stored in a −80° C. freezer.

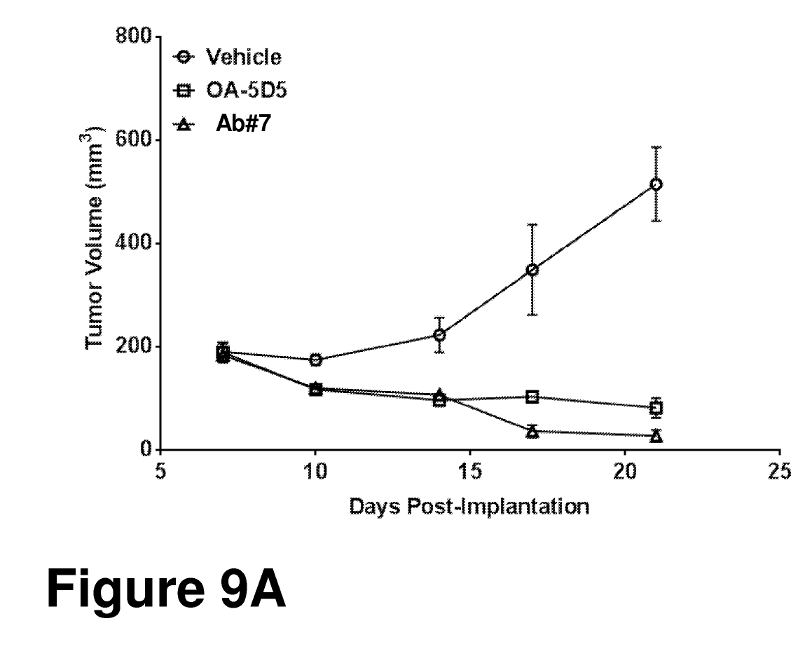

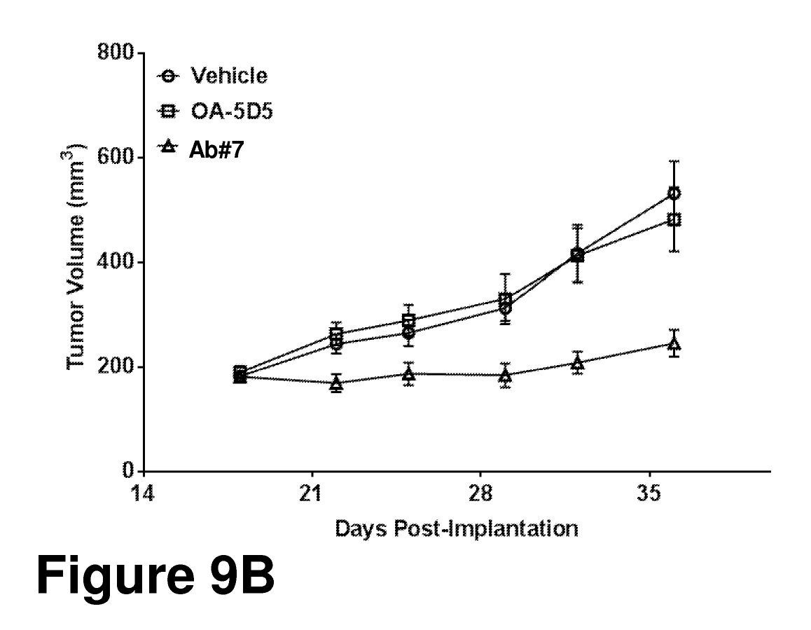

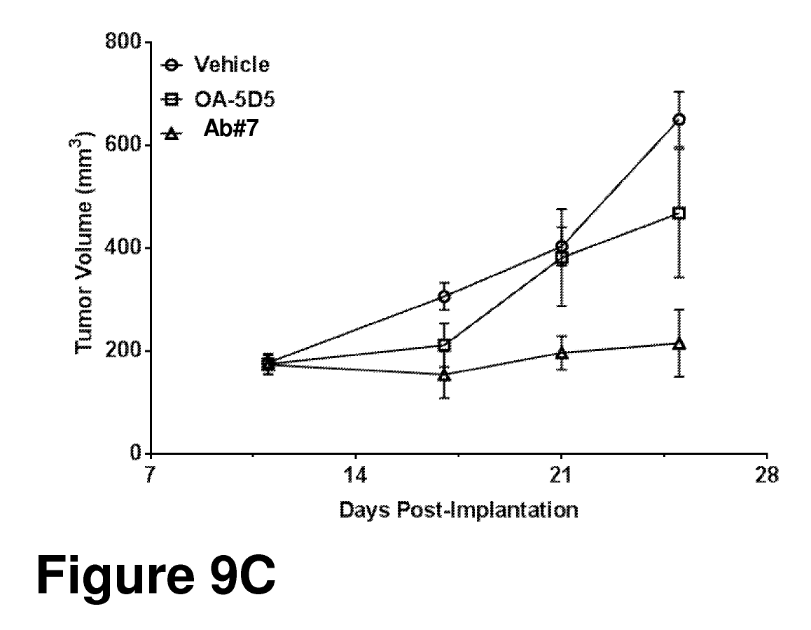

Data from representative experiments using the above protocol are shown in FIG. 9. Mice were inoculated with U87-MG cells (FIG. 9A), H441 cells (FIG. 9B), or HCC827-HGF cells that stably express human HGF (FIG. 9C) and treated with OA-5D5, Ab#7, and control (“Vehicle”) as described above. At the equimolar dosing levels of 10 mg/kg and 12 mg/kg of OA-5D5, and Ab#7, respectively, the bispecific antibody resulted in significantly greater inhibition of tumor growth than OA-5D5. These results show that the bispecific antibody activity is driven by c-Met, HGF, and EpCAM expression.

### Example 3: Measurement of HGF mRNA in Formalin Fixed, Paraffin Embedded Cell Lines

A chromogenic RNA-ISH assay is used to stain an FFPE tissue section for a target mRNA of interest. For each RNA-ISH assay, a scoring system is applied by a certified pathologist. The system scores are discrete variables: 0, 1+, 2+, 3+, or 4+ as set forth in Table 3.

TABLE 3
Ordinal scoring approach for HGF RNA ISH

| Staining | |
|---|---|
| Score | Microscope objective scoring* |
| 0 | No staining or less than 1 dot per 10 cells (40x magnification) |
| 1 | 1-3 dots/cell (visible at 20-40x magnification) |
| 2 | 4-10 dots/cell. Very few dot clusters (visible at 20-40x |
| | magnification) |
| 3 | >10 dots/cell. Fewer than 10% positive cells have dot clusters |
| | (visible at 20x magnification) |
| 4 | >10 dots/cell. Greater than 10% positive cells have dot clusters |
| | (visible at 20x magnification) |

*If <5% of cells score 1 and >95% of cells score 0, a score of 0 will be given.
If 5-30% of cells score 1 and >70% of cells score 0, a score of 0.5 will be given.
Scoring performed at 20x magnification.

The quality of mRNA is assessed by staining with a positive control probe (for human cyclophilin B, LS Positive Control Probe-MM-PPIB Catalog#313917) and a negative control probe (for bacterial DapB, LS Negative Control Probe-DapB Catalog #312037). Cyclophilin B (PPIB) is a low-copy (10-20 copies per cell) housekeeping gene that serves as a rigorous test of tissue mRNA integrity. Bacterial DapB is a bacterial gene-specific probe that generates no background signal on properly fixed human tissue. These two controls are used to assess fixation and verify technical accuracy of the method. The assay is optimized for a broad range of cancerous and normal tissues to facilitate interpretation of the staining results by a certified pathologist.

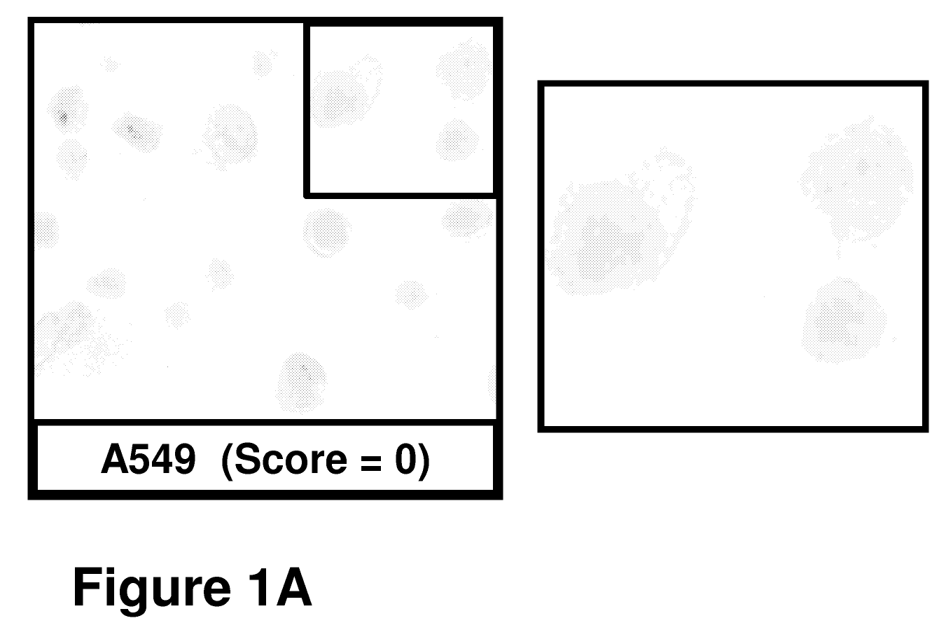

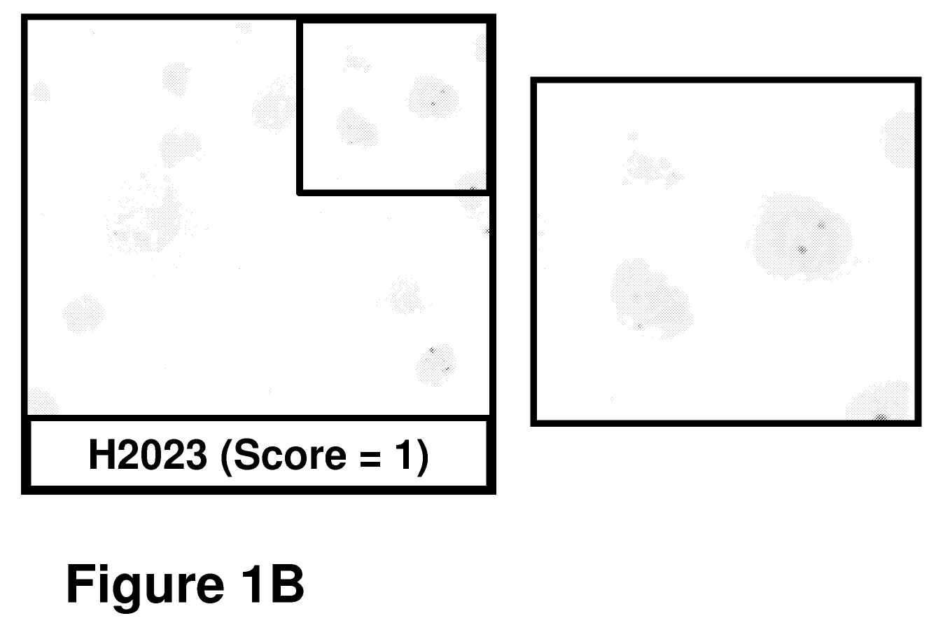

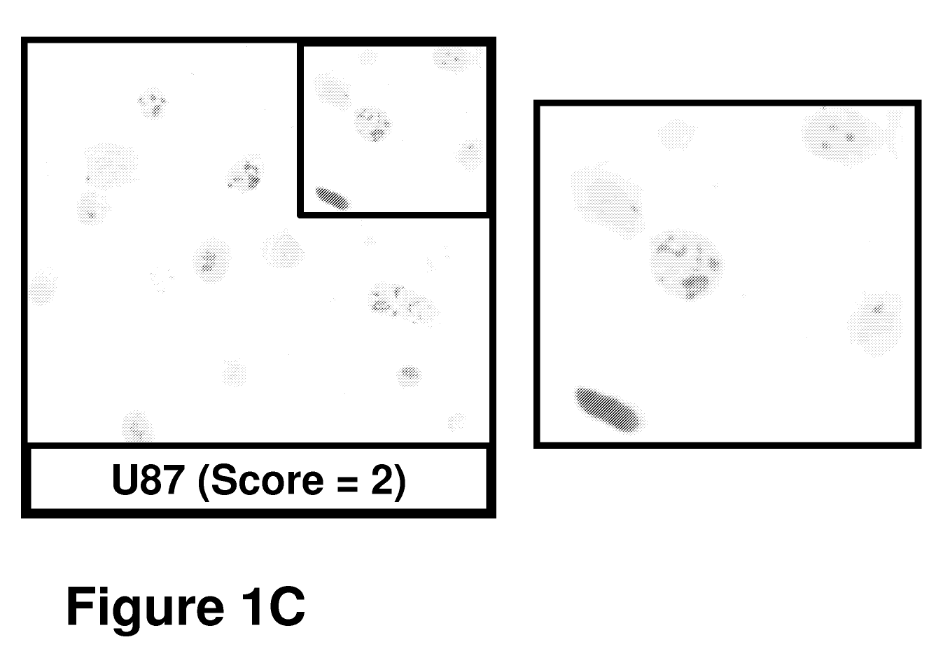

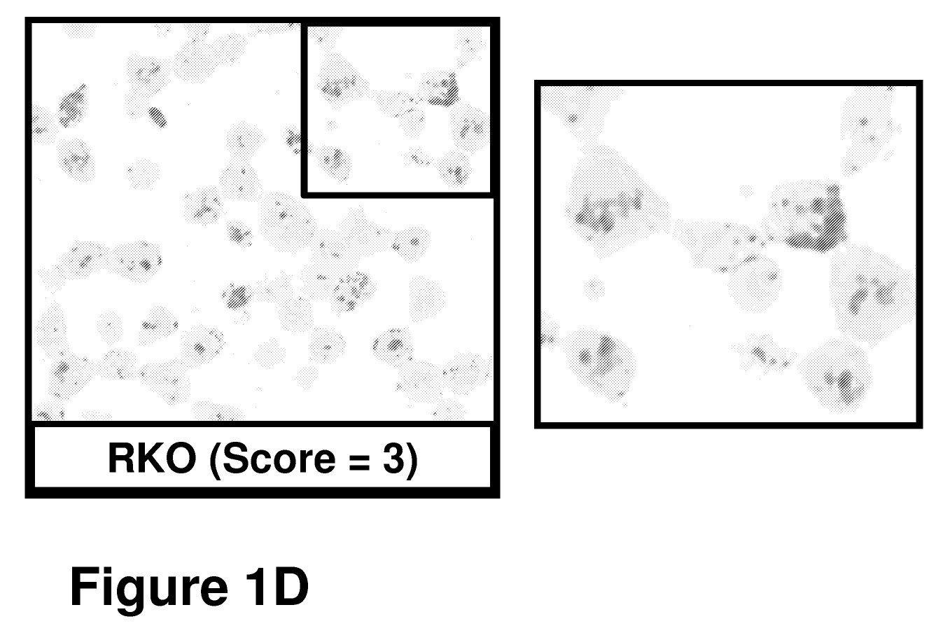

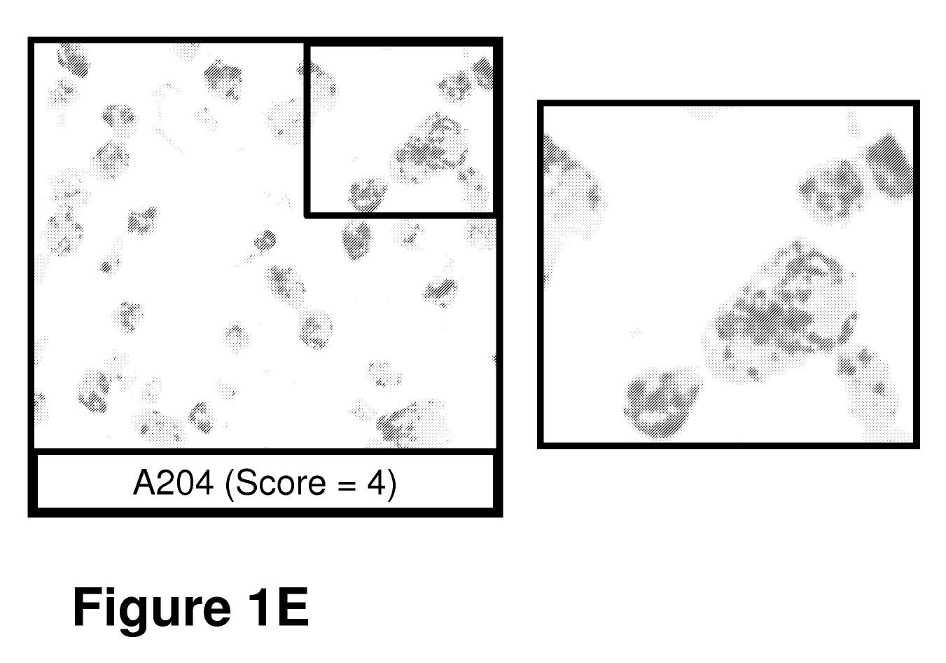

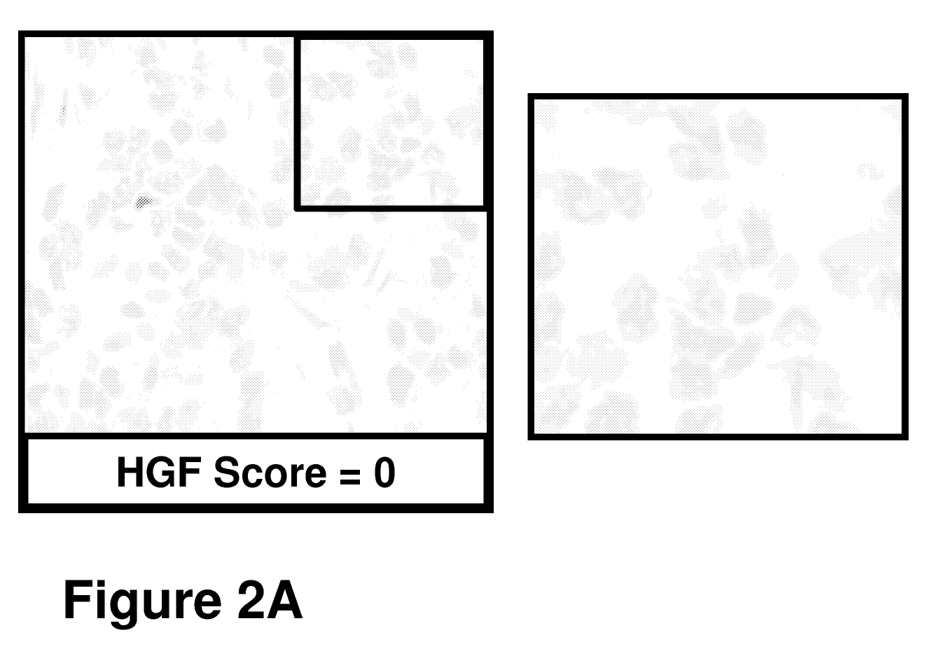

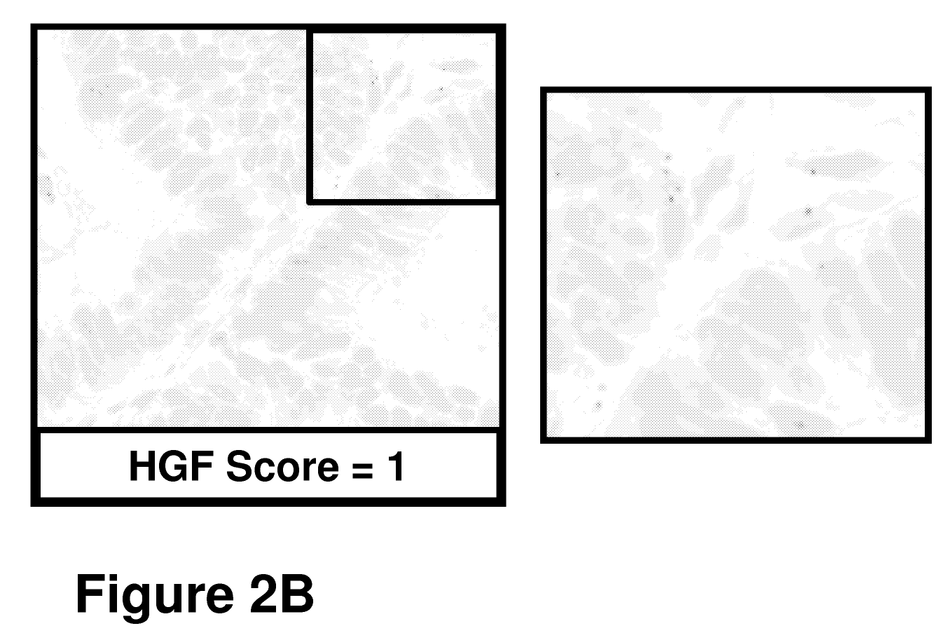

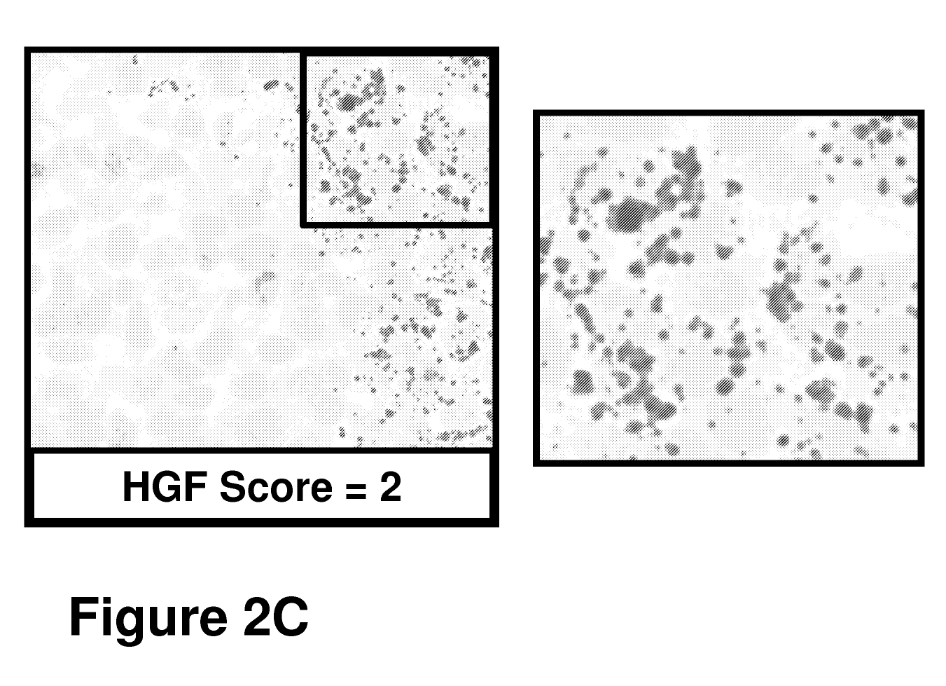

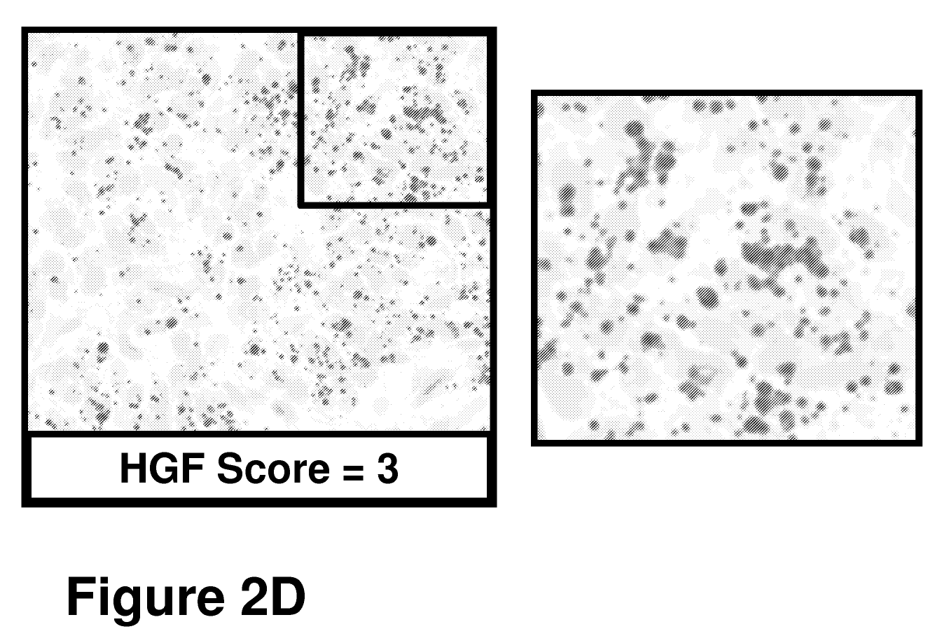

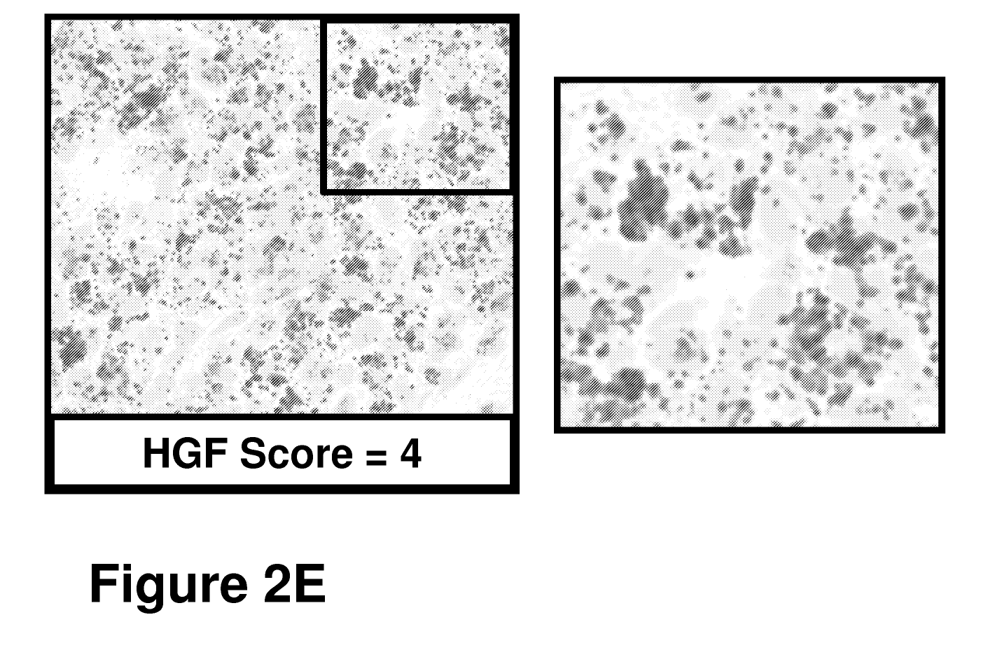

FIG. 1 shows an example of HGF RNA staining in a cell lines exhibiting a range of HGF mRNA expression levels. FIG. 2 shows an example of HGF staining in a human tumor sample. The staining pattern is strikingly non-uniform, with a small subset of cells expressing high levels of HGF and the majority of cells expressing undetectable levels of transcripts.

Detection of HGF mRNA Via RNA ISH:

The detection of HGF mRNA is achieved using the following variant of an Advanced Cell Diagnostics® (“ACD” Hayward, Calif.) RNAscope® assay. In this assay, cells are permeabilized an incubated with a set of oligonucleotide “Z” probes (see, e.g., U.S. Pat. No. 7,709,198) specific for HGF. Using “Z” probes, as well as using multiple sets of probes per transcript, increases the specificity of the assay over standard ISH methods. One HGF probe set that is used in this assay is ACD Part Number 418707 that target a 1460 base long region of the HGF transcript comprising nucleotides 346-1806 of transcript variant 1 mRNA. Following Z probe incubation, a pre-amplifier is added that can only hybridize to a pair of adjacent Z probes bound to the target transcript. This minimizes amplification of non-specific binding. Several sequential amplification steps are then performed based on sequence-specific hybridization to the pre-amplifier, followed by enzyme-mediated chromogenic detection that enables semi-quantitative measurement of HGF RNA levels in the tumor tissue.

Step 1: FFPE sections of cancer cells or tumor tissues are deparaffinized and pretreated to block endogenous phosphatases and peroxidases and to unmask RNA binding sites. Step 2: Target-specific double Z probes are applied, which specifically hybridize to the target RNA at adjacent sequences. Step 3: Targets are detected by sequential applications of a preamplifier oligonucleotide, amplifier oligonucleotides, a final horseradish peroxidase (HRP)-conjugated oligonucleotide, and diaminobenzidine (DAB). Step 4: Slides are visualized using a light microscope and scored by a pathologist.

To score the assay, pathologists are trained using a cell line microarray. These cell lines express different levels of HGF, ranging from undetectable to high, and reflect visually the number of dots expected for each ordinal score (Table 3). A pathologist then assigns the patient sample a score based on visual inspection.

2. Sample Preparation and Staining

Upon biopsy (e.g., by surgical resection), patient tumor samples are immediately placed in fixative (10% neutral buffered formalin) typically for 20-24 hours at room temperature. The samples are then transferred to 70% ethanol and embedded in paraffin as per standard histological procedures to yield FFPE tissue. Before the assay is performed 5+/−1 μm sections of FFPE tissue are prepared and mounted on to positively charged 75×25 mm glass slides. These samples are then stored in a nitrogen chamber to preserve the tissue and target of interest. One of the sections is used for routine H & E staining, which a pathologist reviews for tumor content, quality, and clinical diagnosis. The pathologist differentiates areas of tumor, stroma, and necrosis. Following this review, an adjacent or nearby tissue section (within 20 μm of the H&E section) is used for the assay.

Pretreat solutions, target probes, and wash buffers for RNAscope® assays are obtained from ACD. The assay is run performed following these steps using the following approaches: a) Manual staining, b) automated using a Ventana Medical Systems (Tucson, Ariz.) autostaining platform, or c) automated staining using a Leica Biosystems (Buffalo Grove, Ill.) platform as suggested by the manufacturer, Advanced Cell Diagnostics (ACD), Hayward, Calif. Each approach involves permeabilization of the cells, hybridization of probes to the target, and addition of detection reagents that amplify the signal to allow the target to visualized using standard microscopy techniques. A negative control probe such as bacterial DapB (LS Negative Control Probe-DapB, ACD, Hayward, Calif.; Catalog #312037) is used to demonstrate specific target probe binding and determine that tissue pretreatment doesn't yield false positives. A positive control probe such as human cyclophilin B (PPIB, LS Positive Control Probe-MM-PPIB ACD, Hayward, Calif.; Catalog #313917) is used to help evaluate RNA integrity. An unexpected result in either control probe impacts the ability to interpret the staining pattern of the target probe, HGF (LS Probe-Hs-HGF-v2d ACD, Hayward, Calif.; Catalog #418707). Results shown follow an automated protocol using the reagents listed above on the LEICA BOND Rx, Leica Biosystems. All reagents are purchased from Leica Biosystems or ACD unless otherwise noted.

The following steps are programmed into a LEICA BOND Rx and run as a fully automated assay. The slides are labeled as appropriate when using the LEICA Autostainer software.

First, samples are deparaffinized by baking at 65° as needed (10-30 minutes), followed by dewaxing with LEICA NOVOCASTRA BOND Dewax solution and a series of alcohol washes. After drying, the slides are incubated with LEICA Bond Epitope Retrieval solution 2 at 95° C. for 10-15 minutes and washed with LEICA NOVOCASTRA BOND Wash. Next, the tissues are covered with in the following order: First, Pretreatl solution (ACD), a hydrogen peroxide-based solution that blocks endogenous enzymes, is added and the tissues are incubated for 10-15 minutes at room temperature, and then rinsed twice with LEICA NOVOCASTRA BOND Wash. Slides are then incubated in Pretreat3 (ACD), a protease solution, for 10-15 minutes, which unmasks binding sites, and then rinsed twice with LEICA NOVOCASTRA BOND Wash.

After washing the slides twice with dH₂O, the tissues are covered with the HGF target RNAscope® probes described above for two hours at 40° C. Serial tissue sections are incubated with positive control probes (protein phosphatase 1B (PP1B) ACD Part Number 313917), or negative control probes (bacterial gene DapB-ACD Part Number 312037) for 2 hours at 40° C. Slides are washed twice with 1× RNAscope® wash buffer before incubating with Amp1 reagent for 30 min at 42° C. Amp1 is washed off by a series of three washes with Bond Wash for 3 minutes at room temperature followed by a wash in RNAscope® wash buffer before incubating with ACD 1× LS Wash Buffer is applied and incubated at ambient temperature for 5 minutes at prior to subsequent amplification steps.

Amplification steps consist of incubation in ACD Amp 2 (15 minutes, at 42° C.), ACD Amp 3 (30 minutes at 42° C.), ACD Amp 4 (15 minutes at 42° C.), ACD Amp 5 Brown (30 minutes, at ambient temperature), and ACD Amp 6 Brown (15 minutes at ambient temperature). The final reagent, ACD Amp6, is conjugated to HRP. To visualize the transcripts, the slides are then incubated with ACD staining reagent, which contains DAB, for 10 min at room temperature. Chromogen development is stopped by rinsing with dH₂O. Nuclei are then counterstained with hematoxylin blued with dilute ammonium chloride.

Once the run is complete, slides are removed as soon as possible from the machine. At that time they are dehydrated through a series of increasing percentages of alcohols or alcohol substitutes for at least two minutes each, followed by incubation in at least two dips in xylene for at least 1 minute each.

Scoring of the target probe is performed using image analysis software capable of determining the number of transcripts (that are visible by microscopy as dots) in a cell or by a trained pathologist who is familiar with the pattern created when the chromogen precipitates at the site of target probe binding. A cell pellet array is used as a control. As shown in FIG. 1, A549 is assigned a score of 0, H2023 a score of 1, U-87MG a score of 2, RKO a score of 3, and A204 a score of 4. A representative score is generated for both the tumor and stromal cells found in the tissue sample. The pathologist can assign an ordinal score based on the highest score observed as described above. Scores are provided for the target probe (HGF), as well as the positive control probe (PPIB) and the negative control probe (DapB). A representative image of detection of HGF by RNA ISH in human samples is observed in FIG. 2.

Sensitivity and specificity of the target probe was achieved by comparing the staining pattern for HGF in FFPE sections of cell lines that express a broad range of HGF based on RT-PCR.

Thus in this example, HGF mRNA is detected by ISH by providing a sample of cells or tissue that are fixed and embedded in a medium, such as paraffin. The sample is section and the embedded medium removed, such as by deparaffinization. After blocking endogenous enzymes that may produce unacceptable background signal (such as phosphatases and peroxidases when using HRP as a detection agent), and RNA binding sites unmasked, double Z probes are applied that specifically hybridize to the target RNA, such as HGF RNA. The targets are then detected and scored.

### Example 4: Detection of c-MET, HGF and EpCAM Protein by IHC

The detection of c-Met, HGF and EpCAM protein is performed manually or on any autostainer platform such as that offered by Leica Biosystems, Ventana Medical Systems, or DAKO. Each approach involves removing excess paraffin by baking freshly 5+/−1 μm sections of FFPE tissue requires at 60° C. for 10 minutes, followed by a series of steps to deparaffinize and hydrate sections. Traditionally this is achieved by multiple washes with xylenes, 100% ethanol, into reducing percentages of ethanol, and ultimately washes with water. Alternatively, dewax solutions including, but not limited to, NOVOCASTRA BOND Dewax solution (Leica Biosystems, Catalog # AR9222), Dewax Solution Kit (Biocare Medical, Concord, Calif., Catalog # ORI 6004K T70), DISCOVERY 10× EZ Prep Solution (Ventana Medical Systems, Inc., Catalog #950-100), is used. Once the tissue has been hydrated, antigen unmasking is performed following by staining protocols. Antigen retrieval of c-Met, HGF and EpCAM is achieved using a citrate based pH 6.0 solution at a sub-boiling temperature for 10 minutes.

The protocol described herein is performed on a LEICA BOND Rx machine, and is intended for chromogenic detection of c-MET, HGF and EpCAM protein expression; however, other approaches may yield similar results with slight modifications. Detection of each target is achieved through use of the reagents included in the Refine Kit 2 from Leica Biosystems.

Staining for c-MET:
Step 1: Peroxide Block for 5 minutes at ambient temperature
Steps 2-4: Bond Wash Solution is applied 3 times
Step 5: Primary antibody incubation for 60 minutes at ambient temperature Primary antibody to Met (D1C2) XP® Rabbit mAb (Cell Signaling, Danvers, Mass.; Cat#8198) used at 1:300 dilution
Steps 6-8: Bond Wash Solution is applied 3 times
Step 9: Polymer is added for 8 minutes at ambient temperature
Steps 10-14: Bond Wash Solution is applied 5 times at ambient temperature
Step 15: Deionized Water is applied at ambient temperature
Step 16: Mixed DAB Refine is applied
Step 17: Mixed DAB Refine is applied for 10 minutes
Step 18-20: Deionized Water is applied at ambient temperature
Step 21: Hematoxylin is added and incubated for 5 minutes at ambient temperature
Step 22: Deionized Water is applied at ambient temperature
Step 23: Bond Wash Solution is applied
Step 24: Deionized Water is applied at ambient temperature
   Scoring of a sample stained for the detection of c-Met is performed using image analysis software capable of determining the number of staining intensity in a cell or by a trained pathologist who is familiar with the pattern created when the chromogen precipitates at the site of antibody binding. A cell pellet array is used as a control. As shown in FIG. 3, TOV-112D is assigned a score of 0, H1703 a score of 1, A549 a score of 2, and H441 a score of 3. A representative image of detection of c-MET by IHC in human samples is observed in FIG. 4.

### Staining for HGF:

Step 1: Peroxide Block for 5 minutes at ambient temperature
Steps 2-4: Bond Wash Solution is applied 3 times
Step 5: Primary antibody incubation for 60 minutes at ambient temperature Primary antibody to HGF 0 (D6S7D) XP® Rabbit mAb (Cell Signaling, Danvers, Mass.; Cat#52445) used at 1:50 dilution
Steps 6-8: Bond Wash Solution is applied 3 times
Step 9: Polymer is added for 8 minutes at ambient temperature
Steps 10-14: Bond Wash Solution is applied 5 times at ambient temperature
Step 15: Deionized Water is applied at ambient temperature
Step 16: Mixed DAB Refine is applied
Step 17: Mixed DAB Refine is applied for 10 minutes
Step 18-20: Deionized Water is applied at ambient temperature
Step 21: Hematoxylin is added and incubated for 5 minutes at ambient temperature
Step 22: Deionized Water is applied at ambient temperature
Step 23: Bond Wash Solution is applied
Step 24: Deionized Water is applied at ambient temperature
   Scoring of a sample stained for the detection of HGF is performed using image analysis software capable of determining the number of staining intensity in a cell or by a trained pathologist who is familiar with the pattern created when the chromogen precipitates at the site of antibody binding. A cell pellet array is used as a control. As shown in FIG. 5, A549 is assigned a score of 0, H2023 a score of 1, U87 a score of 2, KP4 a score of 3, and HCC827-HGF a score of 4. A representative image of detection of HGF by IHC in human non-small cell lung cancer (NSCLC) tissue samples is observed in FIG. 6A-C.

### Staining for EpCAM:

Step 1: Peroxide Block for 5 minutes at ambient temperature
Steps 2-4: Bond Wash Solution is applied 3 times
Step 5: Primary antibody incubation for 60 minutes at ambient temperature Primary antibody to EpCAM (VU1D9) Mouse mAb (Cell Signaling, Danvers, Mass.; Cat# #2929) used at 1:250 dilution
Steps 6-8: Bond Wash Solution is applied 3 times
Step 9: Post-Primary Reagent is added for 8 minutes at room temperature
Steps 10-12: Bond Wash Solution is applied 3 times
Step 13: Polymer is added for 8 minutes at ambient temperature
Steps 14-18: Bond Wash Solution is applied 5 times at ambient temperature
Step 19: Deionized Water is applied at ambient temperature
Step 20: Mixed DAB Refine is applied
Step 21: Mixed DAB Refine is applied for 10 minutes
Steps 22-24: Deionized Water is applied at ambient temperature
Step 25: Hematoxylin is added and incubated for 5 minutes at ambient temperature
Step 26: Deionized Water is applied at ambient temperature
Step 27: Bond Wash Solution is applied
Step 28: Deionized Water is applied at ambient temperature

Slides are then de-paraffinized and mounted as described in the preceding Example.

Cell lines stained for EpCAM detection are used to guide interpretation of staining. As shown in FIG. 7, A549 is assigned a score of 0, ACHN a score of 1, SK-OV-3 a score of 2, and H1993 a score of 3. A representative image of detection of EpCAM by IHC in human samples is observed in FIG. 8.

Thus in this example, samples are provided that are prepared for immunohistochemistry (fixed and embedded cells and/or tissue), the samples processed and sectioned, the embedding media removed (such as paraffin), specific antibodies for the target molecule applied and detected, and the samples then scored.

### Equivalents and Incorporation by Reference

Those skilled in the art will recognize, or be able to ascertain and implement using no more than routine experimentation, many equivalents of the specific embodiments described herein. Such equivalents are intended to be encompassed by the following claims. Each combination of the embodiments disclosed in any combination of the dependent claims is contemplated to be within the scope of the disclosure. The disclosure of each and every U.S. and foreign patent and pending patent application and publication referred to herein is specifically incorporated by reference herein in its entirety for all purposes.

1. A method for selecting therapy for, or for providing treatment to, a patient having a cancer, the method comprising:
(a) obtaining at least one biomarker score from at least one cancer cell sample from the patient, wherein the at least one biomarker score comprises a score for one of c-Met and HGF; and
(b) if each score meets a threshold, then
1) selecting the patient for treatment with, or
2) administering to the patient,
an effective amount of a bispecific anti-EpCAM/anti-c-Met antibody,
wherein the threshold comprises only one of:
i. an immunohistochemistry (IHC) score for c-Met of 2+ or higher; or
ii. a c-Met IHC score of 3+;
iii. an HGF RNA in situ hybridization (ISH) score of 1+ or higher;
iv. an HGF RNA ISH score of 2+ or higher;
v. an HGF RNA ISH score of 3+ or higher;
vi. an HGF RNA ISH score of 4+
vii. an HGF IHC score of 1+ or higher;
viii. an HGF IHC score of 2+ or higher;
ix. an HGF IHC score of 3+ or higher; or
x. an HGF IHC score of 4+.

2. A method for selecting therapy for, or for providing treatment to, a patient having a cancer, the method comprising:
(a) obtaining at least two biomarker scores from at least one cancer cell sample from the patient, wherein the scored biomarkers comprise c-Met and HGF; and
(b) if the scores meet a threshold, then
1) selecting the patient for treatment with, or
2) administering to the patient,
an effective amount of a bispecific anti-EpCAM/anti-c-Met antibody,
wherein the threshold comprises only one of:
i. an HGF (by RNA ISH or IHC) score of 1+ or higher and a c-Met IHC score of 1+ or higher;
ii. an HGF (by RNA ISH or IHC) score of 1+ or higher and a c-Met IHC score of 2+ or higher;
iii. an HGF (by RNA ISH or IHC) score of 1+ or higher and a c-Met IHC score of 3+;
iv. an HGF (by RNA ISH or IHC) score of 2+ or higher and a c-Met IHC score of 1+ or higher;
v. an HGF (by RNA ISH or IHC) score of 2+ or higher and a c-Met IHC score of 2+ or higher;
vi. an HGF (by RNA ISH or IHC) score of 2+ or higher and a c-Met IHC score of 3+;
vii. an HGF (by RNA ISH or IHC) score of 3+ or higher and a c-Met IHC score of 1+ or higher;
viii. an HGF (by RNA ISH or IHC) score of 3+ or higher and a c-Met IHC score of 2+ or higher;
ix. an HGF (by RNA ISH or IHC) score of 3+ or higher and a c-Met IHC score of 3+;
x. an HGF (by RNA ISH or IHC) score of 4+ and a c-Met IHC score of 1+ or higher;
xi. an HGF (by RNA ISH or IHC) score of 4+ and a c-Met IHC score of 2+ or higher;
xii. an HGF (by RNA ISH or IHC) score of 4+ and a c-Met IHC score of 3+;
xiii. an HGF (by RNA ISH or IHC) score of 0 and a c-Met IHC score of 2+; or
xiv. an HGF (by RNA ISH or IHC) score of 0 and a c-Met IHC score of 3+.

3. The method of claim 1 or claim 2, further comprising obtaining an EpCAM IHC biomarker score from the cancer cell sample (i.e., from cancer cells within the sample), wherein the threshold further comprises an EpCAM IHC score of 1+ or higher or 2+ or higher, or 3+.

4. The method of claim 1, wherein the threshold is a c-Met IHC score of 2+ or higher or a c-MET IHC score of 3+.

5. The method of claim 1, wherein the threshold is an HGF (by RNA ISH or IHC) score of 1+ or higher.

6. The method of claim 2, wherein the threshold is a c-Met IHC score of 2+ or higher and an HGF RNA-ISH score of 1+ or higher.

7. The method of any of the preceding claims, wherein the bispecific antibody is Ab#5, Ab#7, or Ab#13.

8. The method of any of the preceding claims, wherein the HGF RNA-ISH or IHC score is obtained using one or more nucleic acid ISH probes that hybridize specifically to a nucleic acid that comprises the sequence of nucleotides 346-1806 of the nucleotide sequence set forth in GenBank accession number NM_000601.4.

9. The method of any of the preceding claims, wherein the c-Met IHC scores are obtained using at least one anti-c-Met antibody selected from clone SP44, Clone Met4, and Clone Met (D1C2) XP

10. The method of any of the preceding claims, wherein the cancer is bladder, breast, cervical, colorectal, gastric, gastroesophageal, esophageal, head and neck, liver, lung, ovarian, pancreatic, prostrate, renal or thyroid cancer.

11. The method of any of claims 3 and 7-10, wherein the EpCAM IHC scores are obtained using at least one anti-EpCAM antibody selected from clone VU1D9 and Clone MOC-31.

12. The method of any of the preceding claims, wherein if each score does not meet the threshold, then the patient receives therapy with an anti-cancer therapeutic that does not comprise a bispecific anti-EpCAM/anti-c-Met antibody.

13. The method of any of the preceding claims, wherein the treatment, when administered to a plurality of the selected patients, produces an increase in the frequency in the treated patients of at least one therapeutic effect selected from the group consisting of reduction in size of a tumor, reduction in number of metastatic lesions over time, complete response, partial response, stable disease, increase in overall response rate, or a pathologic complete response, compared to a comparator population of patients who receive the treatment without the selection.

14. The method of any of the preceding claims, wherein the treatment, when administered to a plurality of the selected patients, results in an increase in rates in the treated patients of progression-free survival or overall survival compared to a comparator population of patients who receive the treatment without the selection.

15. The method of any of the preceding claims, wherein the treatment, when administered to a plurality of the selected patients, results in an improved quality of life for the selected patients compared to a comparator population of patients who receive the treatment without the selection.
